SEQUENCE LISTING (1265 sequences)

SEQ ID NO: 1 - DNA, 8100 bp, M13 bacteriophage
agcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctcaactgtgaggaggctcacggacgcgaagaacaggcacgcgtgctggcagaaacccccggtatgaccgtgaaaacggcccgccgcattctggccgcagcaccacagagtgcacaggcgcgcagtgacactgcgctggatcgtctgatgcagggggcaccggcaccgctggctgcaggtaacccggcatctgatgccgttaacgatttgctgaacacaccagtgtaagggatgtttatgacgagcaaagaaacctttacccattaccagccgcagggcaacagtgacccggctcataccgcaaccgcgcccggcggattgagtgcgaaagcgcctgcaatgaccccgctgatgctggacacctccagccgtaagctggttgcgtgggatggcaccaccgacggtgctgccgttggcattcttgcggttgctgctgaccagaccagcaccacgctgacgttctacaagtccggcacgttccgttatgaggatgtgctctggccggaggctgccagcgacgagacgaaaaaacggaccgcgtttgccggaacggcaatcagcatcgtttaactttacccttcatcactaaaggccgcctgtgcggctttttttacgggatttttttatgtcgatgtacacaaccgcccaactgctggcggcaaatgagcagaaatttaagtttgatccgctgtttctgcgtctctttttccgtgagagctatcccttcaccacggagaaagtctatctctcacaaattccgggactggtaaacatggcgctgtacgtttcgccgattgtttccggtgaggttatccgttcccgtggcggctccacctctgaatttacgccgggatatgtcaagccgaagcatgaagta

SEQ ID NO: 2 - DNA, 35 bp, Artificial Sequence
  Description of Artificial Sequence Synthetic polynucleotide
ctttattcgtaaaactagcatattcaaccgttcta

SEQ ID NO: 3 - DNA, 35 bp, Artificial Sequence
  Description of Artificial Sequence Synthetic polynucleotide
agggtgagaaaggcaagaattagcaaaaatcggtt

SEQ ID NO: 4 - DNA, 42 bp, Artificial Sequence
  Description of Artificial Sequence Synthetic polynucleotide
gctgataaattaatgagcataaagctaattaagcaataaagc

SEQ ID NO: 5 - DNA, 42 bp, Artificial Sequence
  Description of Artificial Sequence Synthetic polynucleotide
cgatgaacggtaattcaacgcaaggataggtaaagattcaaa

SEQ ID NO: 6 - DNA, 42 bp, Artificial Sequence
  Description of Artificial Sequence Synthetic polynucleotide
ctaattgggcttgactgagtaatgtgtaaaaatttttagaac

SEQ ID NO: 7 - DNA, 42 bp, Artificial Sequence
  Description of Artificial Sequence Synthetic polynucleotide
gaacgagtagtacagccggagagggtaggcaaacaagagaat

SEQ ID NO: 8 - DNA, 42 bp, Artificial Sequence
  Description of Artificial Sequence Synthetic polynucleotide
cctcatatatttgacctgagagtctggactatttttgagaga

SEQ ID NO: 9 - DNA, 42 bp, Artificial Sequence
  Description of Artificial Sequence Synthetic polynucleotide
aataaatgcaatgcgatggtttaatttcgacgagaaacacca

SEQ ID NO: 10 - DNA, 42 bp, Artificial Sequence
  Description of Artificial Sequence Synthetic polynucleotide
tctacaaaggctacataaggcttgccctaactttaatcattg

SEQ ID NO: 11 - DNA, 42 bp, Artificial Sequence
  Description of Artificial Sequence Synthetic polynucleotide
gtatcaggtcattgggacagatgaacggctgaccaactttga

SEQ ID NO: 12 - DNA, 42 bp, Artificial Sequence
  Description of Artificial Sequence Synthetic polynucleotide
tgaattaccttatgataagggaaccgaatgtacagaccaggc

SEQ ID NO: 13 - DNA, 42 bp, Artificial Sequence
  Description of Artificial Sequence Synthetic polynucleotide
tgctcattcagtgaaacggagatttgtaaagcgcgaaacaaa

SEQ ID NO: 14 - DNA, 42 bp, Artificial Sequence
  Description of Artificial Sequence Synthetic polynucleotide
gcataggctggctgccagcgattatacctcatcgcctgataa

SEQ ID NO: 15 - DNA, 42 bp, Artificial Sequence
  Description of Artificial Sequence Synthetic polynucleotide
gcagacggtcaatccgattttaagaactcaacgtaacaaagc

SEQ ID NO: 16 - DNA, 35 bp, Artificial Sequence
  Description of Artificial Sequence Synthetic polynucleotide
attgtgtcgaaatcttcattacccaaatggctcat

SEQ ID NO: 17 - DNA, 35 bp, Artificial Sequence
  Description of Artificial Sequence Synthetic polynucleotide
ttgacccaccttcatcaagagagccggaacgaggc

SEQ ID NO: 18 - DNA, 35 bp, Artificial Sequence
  Description of Artificial Sequence Synthetic polynucleotide
agatacaccttattagcgtttaaggccggaaacgt

SEQ ID NO: 19 - DNA, 35 bp, Artificial Sequence
  Description of Artificial Sequence Synthetic polynucleotide
gaattagagccagcaaaaatctacgttaggtagaa

SEQ ID NO: 20 - DNA, 42 bp, Artificial Sequence
  Description of Artificial Sequence Synthetic polynucleotide
caccaatgaaaccaacaacattattacaataaaacgaactaa

SEQ ID NO: 21 - DNA, 42 bp, Artificial Sequence
  Description of Artificial Sequence Synthetic polynucleotide
ttcggtcatagccctaacgccaaaaggacttgagccatttgg

SEQ ID NO: 22 - DNA, 42 bp, Artificial Sequence
  Description of Artificial Sequence Synthetic polynucleotide
cgtaaacagttcagatcaccgtcaccgaattacgaggcatag

SEQ ID NO: 23 - DNA, 42 bp, Artificial Sequence
  Description of Artificial Sequence Synthetic polynucleotide
cctcaaatgcttgatcgatagcagcaccttttcatcggcatt

SEQ ID NO: 24 - DNA, 42 bp, Artificial Sequence
  Description of Artificial Sequence Synthetic polynucleotide
taagagcaacactacagactgtagcgcggtaatcagtagcga

SEQ ID NO: 25 - DNA, 42 bp, Artificial Sequence
  Description of Artificial Sequence Synthetic polynucleotide
attaaaggtgaattaaaacgagaatgacattcattgaatccc

SEQ ID NO: 26 - DNA, 42 bp, Artificial Sequence
  Description of Artificial Sequence Synthetic polynucleotide
cagaatcaagttcagaatcgtcataaatcataaatcaaaaat

SEQ ID NO: 27 - DNA, 42 bp, Artificial Sequence
  Description of Artificial Sequence Synthetic polynucleotide
actgcctttagcgttcataaccctcgttgacggaaattattc

SEQ ID NO: 28 - DNA, 42 bp, Artificial Sequence
  Description of Artificial Sequence Synthetic polynucleotide
caggtctttaccctggaaggtaaatatttaccagacgacgat

SEQ ID NO: 29 - DNA, 42 bp, Artificial Sequence
  Description of Artificial Sequence Synthetic polynucleotide
cgtccaatactgcgcggaataagtttatagaaacgcaaagac

SEQ ID NO: 30 - DNA, 42 bp, Artificial Sequence
  Description of Artificial Sequence Synthetic polynucleotide
aaaaaccaaaatagtggcaacatataaatttgtcacaatcaa

SEQ ID NO: 31 - DNA, 42 bp, Artificial Sequence
  Description of Artificial Sequence Synthetic polynucleotide
accgattgagggaggactattatagtcatttagactggatag

SEQ ID NO: 32 - DNA, 35 bp, Artificial Sequence
  Description of Artificial Sequence Synthetic polynucleotide
tagaaaattcatatgtaatagtaaaatggaagcaa

SEQ ID NO: 33 - DNA, 35 bp, Artificial Sequence
  Description of Artificial Sequence Synthetic polynucleotide
ataaaggcgagaggcttttgcaagggcgacattca

SEQ ID NO: 34 - DNA, 35 bp, Artificial Sequence
  Description of Artificial Sequence Synthetic polynucleotide
ctttaatagagtcaatagtgagaaacagtacataa

SEQ ID NO: 35 - DNA, 35 bp, Artificial Sequence
  Description of Artificial Sequence Synthetic polynucleotide
attaattacatttagaggaagcccgaaaaaagcga

SEQ ID NO: 36 - DNA, 42 bp, Artificial Sequence
  Description of Artificial Sequence Synthetic polynucleotide
atcaatatatgtgattaattcgagcttcgacttcaaatatcg

SEQ ID NO: 37 - DNA, 42 bp, Artificial Sequence
  Description of Artificial Sequence Synthetic polynucleotide
ttaagacgctgagatgctccttttgataatcaagaaaacaaa

SEQ ID NO: 38 - DNA, 42 bp, Artificial Sequence
  Description of Artificial Sequence Synthetic polynucleotide
cggtttagctatatatgatgaaacaaacagaggtcatttttg

SEQ ID NO: 39 - DNA, 42 bp, Artificial Sequence
  Description of Artificial Sequence Synthetic polynucleotide
ggtcaataacctttgtgaataaccttgcagcgatagcttaga

SEQ ID NO: 40 - DNA, 42 bp, Artificial Sequence
  Description of Artificial Sequence Synthetic polynucleotide
cggatggcttagagatccttgaaaacatttctgtaaatcgtc

SEQ ID NO: 41 - DNA, 42 bp, Artificial Sequence
  Description of Artificial Sequence Synthetic polynucleotide
ctgagcaaaagaagtttcatttggggcgtacatttcgcaaat

SEQ ID NO: 42 - DNA, 42 bp, Artificial Sequence
  Description of Artificial Sequence Synthetic polynucleotide
gctattaattaaacgtttgaccattagacgagctgaaaaggt

SEQ ID NO: 43 - DNA, 42 bp, Artificial Sequence
  Description of Artificial Sequence Synthetic polynucleotide
tattttcccttagacttaattgctgaatttcatttcaattac

SEQ ID NO: 44 - DNA, 42 bp, Artificial Sequence
  Description of Artificial Sequence Synthetic polynucleotide
ggcatcaattctacgcagaggcgaattaataatgctgtagct

SEQ ID NO: 45 - DNA, 42 bp, Artificial Sequence
  Description of Artificial Sequence Synthetic polynucleotide
aacgagtagatttagtcagatgaatatatagattttcaggtt

SEQ ID NO: 46 - DNA, 42 bp, Artificial Sequence
  Description of Artificial Sequence Synthetic polynucleotide
caacatgttttaaaataaagaaattgcgcagtaacagtacct

SEQ ID NO: 47 - DNA, 42 bp, Artificial Sequence
  Description of Artificial Sequence Synthetic polynucleotide
agttacaaaatcgctaatagtagtagcattcccaattctgcg

SEQ ID NO: 48 - DNA, 35 bp, Artificial Sequence
  Description of Artificial Sequence Synthetic polynucleotide
tttacatcgggagacatataacagttgattaacat

SEQ ID NO: 49 - DNA, 35 bp, Artificial Sequence
  Description of Artificial Sequence Synthetic polynucleotide
aacagaatatgcaactaaagttgctttgaatacca

SEQ ID NO: 50 - DNA, 35 bp, Artificial Sequence
  Description of Artificial Sequence Synthetic polynucleotide
gtctcgtgggttagaacctaccattttgcggaaca

SEQ ID NO: 51 - DNA, 35 bp, Artificial Sequence
  Description of Artificial Sequence Synthetic polynucleotide
ctttgcccgaacgtaaatcccgtaaaaataaagtt

SEQ ID NO: 52 - DNA, 42 bp, Artificial Sequence
  Description of Artificial Sequence Synthetic polynucleotide
aagaaaccaccagattagtgatgaagggaagccgcacaggcg

SEQ ID NO: 53 - DNA, 42 bp, Artificial Sequence
  Description of Artificial Sequence Synthetic polynucleotide
tctgaataatggaacgctggcagcctccactcgtattaaatc

SEQ ID NO: 54 - DNA, 42 bp, Artificial Sequence
  Description of Artificial Sequence Synthetic polynucleotide
gcgccgggcgcggtcaaacaattcgacaggccagagcacatc

SEQ ID NO: 55 - DNA, 42 bp, Artificial Sequence
  Description of Artificial Sequence Synthetic polynucleotide
cgcactcaatccctaggagcggaattatgtttggattatact

SEQ ID NO: 56 - DNA, 42 bp, Artificial Sequence
  Description of Artificial Sequence Synthetic polynucleotide
ctcataacggaacgatataatcctgattcatcatattcctga

SEQ ID NO: 57 - DNA, 42 bp, Artificial Sequence
  Description of Artificial Sequence Synthetic polynucleotide
agtattagactttatgcggtatgagccgattgcaggcgcttt

SEQ ID NO: 58 - DNA, 42 bp, Artificial Sequence
  Description of Artificial Sequence Synthetic polynucleotide
ttatcagatgatatgcatcagcggggtcggtcactgttgccc

SEQ ID NO: 59 - DNA, 42 bp, Artificial Sequence
  Description of Artificial Sequence Synthetic polynucleotide
atggcaattcatcatgccggacttgtagtttgaggatttaga

SEQ ID NO: 60 - DNA, 42 bp, Artificial Sequence
  Description of Artificial Sequence Synthetic polynucleotide
tgcggctggtaatgcaatagataatacaaacgtcagcgtggt

SEQ ID NO: 61 - DNA, 42 bp, Artificial Sequence
  Description of Artificial Sequence Synthetic polynucleotide
gctggaggtgtccactggtcagttggcacaaaccctcaatca

SEQ ID NO: 62 - DNA, 42 bp, Artificial Sequence
  Description of Artificial Sequence Synthetic polynucleotide
gctggtctggtcagtgaacctcaaatataatcaacagttgaa

SEQ ID NO: 63 - DNA, 42 bp, Artificial Sequence
  Description of Artificial Sequence Synthetic polynucleotide
tagattagagccgtggtaaaggtttcttgcaaccagcttacg

SEQ ID NO: 64 - DNA, 35 bp, Artificial Sequence
  Description of Artificial Sequence Synthetic polynucleotide
aggaattgaggaagtggtgccatcccactgctcgt

SEQ ID NO: 65 - DNA, 35 bp, Artificial Sequence
  Description of Artificial Sequence Synthetic polynucleotide
accttgccagcaaccgcaagagcactaacaactaa

SEQ ID NO: 66 - DNA, 35 bp, Artificial Sequence
  Description of Artificial Sequence Synthetic polynucleotide
attatttcgctgagagccagctagtctttaatgcg

SEQ ID NO: 67 - DNA, 35 bp, Artificial Sequence
  Description of Artificial Sequence Synthetic polynucleotide
aagcgtaagaatactgctggtaatatccaaaaacg

SEQ ID NO: 68 - DNA, 42 bp, Artificial Sequence
  Description of Artificial Sequence Synthetic polynucleotide
cgaactgatagcccgccattgcaacaggagaacaatattacc

SEQ ID NO: 69 - DNA, 42 bp, Artificial Sequence
  Description of Artificial Sequence Synthetic polynucleotide
ctgcaacagtgccaacattggcagattccccttctgacctga

SEQ ID NO: 70 - DNA, 42 bp, Artificial Sequence
  Description of Artificial Sequence Synthetic polynucleotide
gcaatttaggcagacaacagagatagaaaccagtcacacgac

SEQ ID NO: 71 - DNA, 42 bp, Artificial Sequence
  Description of Artificial Sequence Synthetic polynucleotide
acgccaacatgtcataaaacatcgccatagtattaacaccgc

SEQ ID NO: 72 - DNA, 42 bp, Artificial Sequence
  Description of Artificial Sequence Synthetic polynucleotide
cagtaataaaagtggaggtgaggcggtctaaaaataccgaac

SEQ ID NO: 73 - DNA, 42 bp, Artificial Sequence
  Description of Artificial Sequence Synthetic polynucleotide
cgggacattctggcggcattttcgagcccgccatatttaaca

SEQ ID NO: 74 - DNA, 42 bp, Artificial Sequence
  Description of Artificial Sequence Synthetic polynucleotide
gaaccaccagcaatgcttaattgagaatagtaataagagaat

SEQ ID NO: 75 - DNA, 42 bp, Artificial Sequence
  Description of Artificial Sequence Synthetic polynucleotide
atgaagataaaacatgataaataaggcgtttgaaataccgac

SEQ ID NO: 76 - DNA, 42 bp, Artificial Sequence
  Description of Artificial Sequence Synthetic polynucleotide
ataaagtaccgacacctaaatttaatggttaaataagaataa

SEQ ID NO: 77 - DNA, 42 bp, Artificial Sequence
  Description of Artificial Sequence Synthetic polynucleotide
cgctcaacagtaggaactatatgtaaatgcttaggttgggtt

SEQ ID NO: 78 - DNA, 42 bp, Artificial Sequence
  Description of Artificial Sequence Synthetic polynucleotide
acaccggaatcatactttttaacctccggctgatgcaaatcc

SEQ ID NO: 79 - DNA, 42 bp, Artificial Sequence
  Description of Artificial Sequence Synthetic polynucleotide
atttcatcttctgaaaaggtaaagtaatcagtataaagccaa

SEQ ID NO: 80 - DNA, 35 bp, Artificial Sequence
  Description of Artificial Sequence Synthetic polynucleotide
aatcgcaagacaaaatacaaattcttactctgtcc

SEQ ID NO: 81 - DNA, 35 bp, Artificial Sequence
  Description of Artificial Sequence Synthetic polynucleotide
agactacattactagaaaaagatatattttagtta

SEQ ID NO: 82 - DNA, 35 bp, Artificial Sequence
  Description of Artificial Sequence Synthetic polynucleotide
aacagctaacgaaagaggcaaagacagcatcggaa

SEQ ID NO: 83 - DNA, 35 bp, Artificial Sequence
  Description of Artificial Sequence Synthetic polynucleotide
gagttaaaggccgcaataattttttcacgagcctt

SEQ ID NO: 84 - DNA, 42 bp, Artificial Sequence
  Description of Artificial Sequence Synthetic polynucleotide
cgagggtagcaacgaaaggctccaaaaggttgaaaatctcca

SEQ ID NO: 85 - DNA, 42 bp, Artificial Sequence
  Description of Artificial Sequence Synthetic polynucleotide
aggcaccaacctaatgataccgatagttctgaggcttgcagg

SEQ ID NO: 86 - DNA, 35 bp, Artificial Sequence
  Description of Artificial Sequence Synthetic polynucleotide
aaccacccctattattctgaacagtaagcgtcata

SEQ ID NO: 87 - DNA, 35 bp, Artificial Sequence
  Description of Artificial Sequence Synthetic polynucleotide
cattaaagccagaatcaccggaaccagacaccctc

SEQ ID NO: 88 - DNA, 42 bp, Artificial Sequence
  Description of Artificial Sequence Synthetic polynucleotide
catggcttttgatgtccctcagagccgcgccaccaccggaac

SEQ ID NO: 89 - DNA, 42 bp, Artificial Sequence
  Description of Artificial Sequence Synthetic polynucleotide
tgcctatttcggaaaccagagccgccgcaacaaataaatcct

SEQ ID NO: 90 - DNA, 35 bp, Artificial Sequence
  Description of Artificial Sequence Synthetic polynucleotide
caataatatcctgaatcttacgatatagaaggctt

SEQ ID NO: 91 - DNA, 35 bp, Artificial Sequence
  Description of Artificial Sequence Synthetic polynucleotide
tttcatcgtaggaatcagctaatgcagagaacaag

SEQ ID NO: 92 - DNA, 42 bp, Artificial Sequence
  Description of Artificial Sequence Synthetic polynucleotide
atccggtattctaaaatagataagtcctacgcgcctgtttat

SEQ ID NO: 93 - DNA, 42 bp, Artificial Sequence
  Description of Artificial Sequence Synthetic polynucleotide
ccagctacaattttcggctgtctttcctcaagccgtttttat

SEQ ID NO: 94 - DNA, 35 bp, Artificial Sequence
  Description of Artificial Sequence Synthetic polynucleotide
caccgccgttttttggggtcggttgagtgttgttc

SEQ ID NO: 95 - DNA, 35 bp, Artificial Sequence
  Description of Artificial Sequence Synthetic polynucleotide
atcggcaaaatcccatgaatcggccaaccagggtg

SEQ ID NO: 96 - DNA, 42 bp, Artificial Sequence
  Description of Artificial Sequence Synthetic polynucleotide
cagtttggaacaagtgcgtattgggcgcgcgcggggagaggc

SEQ ID NO: 97 - DNA, 42 bp, Artificial Sequence
  Description of Artificial Sequence Synthetic polynucleotide
atcacccaaatcaatggccctgagagagatggtggttccgaa

SEQ ID NO: 98 - DNA, 35 bp, Artificial Sequence
  Description of Artificial Sequence Synthetic polynucleotide
cgacgttccagcagttgggcgcgccatgtttacca

SEQ ID NO: 99 - DNA, 35 bp, Artificial Sequence
  Description of Artificial Sequence Synthetic polynucleotide
cgggaacggataacgcgatcggtgcgggggatgtg

SEQ ID NO: 100 - DNA, 42 bp, Artificial Sequence
  Description of Artificial Sequence Synthetic polynucleotide
gtcccggaatttgtagctggcgaaagggcctcttcgctatta

SEQ ID NO: 101 - DNA, 42 bp, Artificial Sequence
  Description of Artificial Sequence Synthetic polynucleotide
ctgctcatttgccggtaaaacgacggccaggtggagccgcca

SEQ ID NO: 102 - DNA, 42 bp, Artificial Sequence
  Description of Artificial Sequence Synthetic polynucleotide
aactttcaacagttccctcagaaccgcccctcattttcaggg

SEQ ID NO: 103 - DNA, 42 bp, Artificial Sequence
  Description of Artificial Sequence Synthetic polynucleotide
cacagacagccctccagtaccaggcggagattagcggggttt

SEQ ID NO: 104 - DNA, 42 bp, Artificial Sequence
  Description of Artificial Sequence Synthetic polynucleotide
atagcaagcccaataagagaaggattagtaagtgccgtcgag

SEQ ID NO: 105 - DNA, 42 bp, Artificial Sequence
  Description of Artificial Sequence Synthetic polynucleotide
gtttagtaccgccatcagcggagtgagacgcctgtagcattc

SEQ ID NO: 106 - DNA, 35 bp, Artificial Sequence
  Description of Artificial Sequence Synthetic polynucleotide
agggttgatataagagtacaaactacaaatagaaa

SEQ ID NO: 107 - DNA, 35 bp, Artificial Sequence
  Description of Artificial Sequence Synthetic polynucleotide
actcctcaggaacccatgtacaccgtactcaggag

SEQ ID NO: 108 - DNA, 42 bp, Artificial Sequence
  Description of Artificial Sequence Synthetic polynucleotide
agaactggcatgatcgcattagacgggaggtaattgagcgct

SEQ ID NO: 109 - DNA, 42 bp, Artificial Sequence
  Description of Artificial Sequence Synthetic polynucleotide
cttaccgaagccctttatcccaatccaaaataaacagccata

SEQ ID NO: 110 - DNA, 42 bp, Artificial Sequence
  Description of Artificial Sequence Synthetic polynucleotide
aatatcagagagattttgccagttacaaataagaaacgattt

SEQ ID NO: 111 - DNA, 42 bp, Artificial Sequence
  Description of Artificial Sequence Synthetic polynucleotide
taaaaacagggaagtaagactccttattatagcaatagctat

SEQ ID NO: 112 - DNA, 35 bp, Artificial Sequence
  Description of Artificial Sequence Synthetic polynucleotide
tttgtttaacgtcacaagaaacaatgaaacgcagt

SEQ ID NO: 113 - DNA, 35 bp, Artificial Sequence
  Description of Artificial Sequence Synthetic polynucleotide
agcctaaaacccacaagaattcagagagaataaca

SEQ ID NO: 114 - DNA, 42 bp, Artificial Sequence
  Description of Artificial Sequence Synthetic polynucleotide
acttctttgattagcaccacacccgccgggttgctttgacga

SEQ ID NO: 115 - DNA, 42 bp, Artificial Sequence
  Description of Artificial Sequence Synthetic polynucleotide
acggtacgccagaaaagccggcgaacgttttagagcttgacg

SEQ ID NO: 116 - DNA, 42 bp, Artificial Sequence
  Description of Artificial Sequence Synthetic polynucleotide
gcacgtataacgtgaagggagcccccgaggcgagaaaggaag

SEQ ID NO: 117 - DNA, 42 bp, Artificial Sequence
  Description of Artificial Sequence Synthetic polynucleotide
acgctgcgcgtaactaataacatcacttgattttagacagga

SEQ ID NO: 118 - DNA, 35 bp, Artificial Sequence
  Description of Artificial Sequence Synthetic polynucleotide
ggaagaaagcgaaaaggccgattaaagggcctgag

SEQ ID NO: 119 - DNA, 35 bp, Artificial Sequence
  Description of Artificial Sequence Synthetic polynucleotide
aaccctactttcctcgttagacaagtgtagcggtc

SEQ ID NO: 120 - DNA, 42 bp, Artificial Sequence
  Description of Artificial Sequence Synthetic polynucleotide
attgcgttgcgctcgggccgttttcacgctgttcttcgcgtc

SEQ ID NO: 121 - DNA, 42 bp, Artificial Sequence
  Description of Artificial Sequence Synthetic polynucleotide
aaattgttatccgcagccagcggtgccgagatgccgggttac

SEQ ID NO: 122 - DNA, 42 bp, Artificial Sequence
  Description of Artificial Sequence Synthetic polynucleotide
cgtgagcctcctcatcgttaacggcatcgtgccccctgcatc

SEQ ID NO: 123 - DNA, 42 bp, Artificial Sequence
  Description of Artificial Sequence Synthetic polynucleotide
ggccagaatgcggcactgcccgctttccctgtttcctgtgtg

SEQ ID NO: 124 - DNA, 35 bp, Artificial Sequence
  Description of Artificial Sequence Synthetic polynucleotide
agacgatccagcgcaatcatggtcatagagtcggg

SEQ ID NO: 125 - DNA, 35 bp, Artificial Sequence
  Description of Artificial Sequence Synthetic polynucleotide
cagcaaacagttgaggatcccctctgtggtgctgc

SEQ ID NO: 126 - DNA, 42 bp, Artificial Sequence
  Description of Artificial Sequence Synthetic polynucleotide
ggtgccggaaaccaaaaaataattcgcgttaaatgtgagcga

SEQ ID NO: 127 - DNA, 42 bp, Artificial Sequence
  Description of Artificial Sequence Synthetic polynucleotide
cgcatcgtaaccgtaaatatttaaattgcaggaagattgtat

SEQ ID NO: 128 - DNA, 42 bp, Artificial Sequence
  Description of Artificial Sequence Synthetic polynucleotide
gtaacaacccgtcgaaaagccccaaaaataaacgttaatatt

SEQ ID NO: 129 - DNA, 42 bp, Artificial Sequence
  Description of Artificial Sequence Synthetic polynucleotide
ataggaacgccatcggcaaagcgccattttggtgtagatggg

SEQ ID NO: 130 - DNA, 35 bp, Artificial Sequence
  Description of Artificial Sequence Synthetic polynucleotide
ttgttaaaattcgctgggataggtcacgcgccatt

SEQ ID NO: 131 - DNA, 35 bp, Artificial Sequence
  Description of Artificial Sequence Synthetic polynucleotide
taatcaggattctccgtgggatcattttttaacca

SEQ ID NO: 132 - DNA, 46 bp, Artificial Sequence
  Description of Artificial Sequence Synthetic polynucleotide
agattcatcagttgagttttttttttttaccattagcgccatcttt

SEQ ID NO: 133 - DNA, 52 bp, Artificial Sequence
  Description of Artificial Sequence Synthetic polynucleotide
tataccagtcagtttttttttggatacgcgatttttttttaaacactcatct

SEQ ID NO: 134 - DNA, 46 bp, Artificial Sequence
  Description of Artificial Sequence Synthetic polynucleotide
taattgtatcggtttattttttttttttagcagcgaaaagaataca

SEQ ID NO: 135 - DNA, 52 bp, Artificial Sequence
  Description of Artificial Sequence Synthetic polynucleotide
ggaacaactaaattttttttttcacctatagttttttttttaagaggctgag

SEQ ID NO: 136 - DNA, 46 bp, Artificial Sequence
  Description of Artificial Sequence Synthetic polynucleotide
agaaccgccaccctcattttttttttttttaccgttcacatgaaag

SEQ ID NO: 137 - DNA, 46 bp, Artificial Sequence
  Description of Artificial Sequence Synthetic polynucleotide
aaaaataatatcccatttttttttttttagcaaatcacaacgctaa

SEQ ID NO: 138 - DNA, 52 bp, Artificial Sequence
  Description of Artificial Sequence Synthetic polynucleotide
agacgacgacaatttttttttgcgttgaacgtttttttttcataggtctgag

SEQ ID NO: 139 - DNA, 46 bp, Artificial Sequence
  Description of Artificial Sequence Synthetic polynucleotide
accagaccggaagcaatttttttttttttttttaatgatttatcaa

SEQ ID NO: 140 - DNA, 52 bp, Artificial Sequence
  Description of Artificial Sequence Synthetic polynucleotide
agcggattgcattttttttttaggggggttttttttttttgaaaatacatac

SEQ ID NO: 141 - DNA, 52 bp, Artificial Sequence
  Description of Artificial Sequence Synthetic polynucleotide
atgttagcaaactttttttttaagagaaaattttttttttgcgtctttccag

SEQ ID NO: 142 - DNA, 46 bp, Artificial Sequence
  Description of Artificial Sequence Synthetic polynucleotide
ctgcaaggcgattaagttttttttttttaacgtacaggttgtgtac

SEQ ID NO: 143 - DNA, 52 bp, Artificial Sequence
  Description of Artificial Sequence Synthetic polynucleotide
caggctgcgcaatttttttttcgtaaattaattttttttttaccccggttga

SEQ ID NO: 144 - DNA, 46 bp, Artificial Sequence
  Description of Artificial Sequence Synthetic polynucleotide
gtaccaaaaacattatttttttttttttcaatatgatgtcaatcat

SEQ ID NO: 145 - DNA, 52 bp, Artificial Sequence
  Description of Artificial Sequence Synthetic polynucleotide
ccaataaatcattttttttttcattcaacaatttttttttatttgcacgtaa

SEQ ID NO: 146 - DNA, 46 bp, Artificial Sequence
  Description of Artificial Sequence Synthetic polynucleotide
aaacgatgctgattgctttttttttttttaacattatcatatcaaa

SEQ ID NO: 147 - DNA, 46 bp, Artificial Sequence
  Description of Artificial Sequence Synthetic polynucleotide
ctcatggaaatacctattttttttttttaatggctatagcaaatga

SEQ ID NO: 148 - DNA, 52 bp, Artificial Sequence
  Description of Artificial Sequence Synthetic polynucleotide
tagaagaactcatttttttttacaggggagctttttttttgcactaaatcgg

SEQ ID NO: 149 - DNA, 46 bp, Artificial Sequence
  Description of Artificial Sequence Synthetic polynucleotide
gtttttcttttcaccattttttttttttcgagataggaggtgccgt

SEQ ID NO: 150 - DNA, 52 bp, Artificial Sequence
  Description of Artificial Sequence Synthetic polynucleotide
aaacctgtcgtgtttttttttttcgtagtgttttttttttcactggtgtgtt

SEQ ID NO: 151 - DNA, 52 bp, Artificial Sequence
  Description of Artificial Sequence Synthetic polynucleotide
cataaacatccctttttttttgtcgggttattttttttttatctaaagcatc

SEQ ID NO: 152 - DNA, 52 bp, Artificial Sequence
  Description of Artificial Sequence Synthetic polynucleotide
gttgggaagaaaatcacctttttactttaatcttttcgaggtgaatttctta

SEQ ID NO: 153 - DNA, 52 bp, Artificial Sequence
  Description of Artificial Sequence Synthetic polynucleotide
attgcgaatttttgcgggttttgtatccgtaatttttcagagccgccaccag

SEQ ID NO: 154 - DNA, 38 bp, Artificial Sequence
  Description of Artificial Sequence Synthetic polynucleotide
taatcaaaatggaaagcgttttcacattcaactaatgc

SEQ ID NO: 155 - DNA, 52 bp, Artificial Sequence
  Description of Artificial Sequence Synthetic polynucleotide
acaacatgttcattaccgttttttcaacctgtttttcaggattagagagtac

SEQ ID NO: 156 - DNA, 32 bp, Artificial Sequence
  Description of Artificial Sequence Synthetic polynucleotide
aaagattaaacaatttcattttgacaaaaaag

SEQ ID NO: 157 - DNA, 30 bp, Artificial Sequence
  Description of Artificial Sequence Synthetic polynucleotide
ctttagagttttttgcatgtagaaaccaat

SEQ ID NO: 158 - DNA, 52 bp, Artificial Sequence
  Description of Artificial Sequence Synthetic polynucleotide
ttgggaaggctcaccggatttttcagcacaaattttcttttgcgggagaagc

SEQ ID NO: 159 - DNA, 52 bp, Artificial Sequence
  Description of Artificial Sequence Synthetic polynucleotide
ggcaaggcacggagacagttttctgatacggtttttcgcggtccgttttttc

SEQ ID NO: 160 - DNA, 38 bp, Artificial Sequence
  Description of Artificial Sequence Synthetic polynucleotide
gacataaaatattaatttttttagggttttcccagtca

SEQ ID NO: 161 - DNA, 52 bp, Artificial Sequence
  Description of Artificial Sequence Synthetic polynucleotide
tatcggcctgtggcacagttttgctggatcagttttacagctgattgccctt

SEQ ID NO: 162 - DNA, 32 bp, Artificial Sequence
  Description of Artificial Sequence Synthetic polynucleotide
gctgcattattataaatcttttgtgcacgggt

SEQ ID NO: 163 - DNA, 30 bp, Artificial Sequence
  Description of Artificial Sequence Synthetic polynucleotide
taggaatgccttttcaatcgtctgaaatgg

SEQ ID NO: 164 - DNA, 42 bp, Artificial Sequence
  Description of Artificial Sequence Synthetic polynucleotide
aattgtagcaatacgatatattcggtcggcgccgacaatgac

SEQ ID NO: 165 - DNA, 42 bp, Artificial Sequence
  Description of Artificial Sequence Synthetic polynucleotide
gcaaattaaccgaagctacagaggcttttaatgccactacga

SEQ ID NO: 166 - DNA, 42 bp, Artificial Sequence
  Description of Artificial Sequence Synthetic polynucleotide
aacaaccatcgcgcacgggtaaaatacggaggactaaagact

SEQ ID NO: 167 - DNA, 42 bp, Artificial Sequence
  Description of Artificial Sequence Synthetic polynucleotide
cgacatcccttacaccttgatattcacacagcattgacagga

SEQ ID NO: 168 - DNA, 42 bp, Artificial Sequence
  Description of Artificial Sequence Synthetic polynucleotide
tgctcgtcataaccatacaggagtgtaccagttaatgccccc

SEQ ID NO: 169 - DNA, 42 bp, Artificial Sequence
  Description of Artificial Sequence Synthetic polynucleotide
ggttgaggcaggtgagtgcccgtataaatggtaataagtttt

SEQ ID NO: 170 - DNA, 42 bp, Artificial Sequence
  Description of Artificial Sequence Synthetic polynucleotide
cattatcaaaatcatcatcgagaacaagtatcattccaagaa

SEQ ID NO: 171 - DNA, 42 bp, Artificial Sequence
  Description of Artificial Sequence Synthetic polynucleotide
tcaatagtgaatacgaacgcgaggcgttttgctattttgcac

SEQ ID NO: 172 - DNA, 42 bp, Artificial Sequence
  Description of Artificial Sequence Synthetic polynucleotide
cgggtattaaactctaaatcaagattagttagcgaacctccc

SEQ ID NO: 173 - DNA, 42 bp, Artificial Sequence
  Description of Artificial Sequence Synthetic polynucleotide
ggtccttattacgcgaaaatcctgtttgttgcagcaagcggt

SEQ ID NO: 174 - DNA, 42 bp, Artificial Sequence
  Description of Artificial Sequence Synthetic polynucleotide
catgattaagacttagtccactattaaaccactacgtgaacc

SEQ ID NO: 175 - DNA, 42 bp, Artificial Sequence
  Description of Artificial Sequence Synthetic polynucleotide
ccacgctggtttgtatcagggcgatggcgaacgtggactcca

SEQ ID NO: 176 - DNA, 42 bp, Artificial Sequence
  Description of Artificial Sequence Synthetic polynucleotide
cgcgatgaacggtacggcgtaaattcagagtgccaagcttac

SEQ ID NO: 177 - DNA, 42 bp, Artificial Sequence
  Description of Artificial Sequence Synthetic polynucleotide
aaacaagagaatccgagagatagacttttcaaacttaaattt

SEQ ID NO: 178 - DNA, 42 bp, Artificial Sequence
  Description of Artificial Sequence Synthetic polynucleotide
ttcatgcttcggaagcagaaacagcggactccgtggtgaagg

SEQ ID NO: 179 - DNA, 42 bp, Artificial Sequence
  Description of Artificial Sequence Synthetic polynucleotide
cagtattaaatcctgaattttctgtatggatctaaagttttg

SEQ ID NO: 180 - DNA, 42 bp, Artificial Sequence
  Description of Artificial Sequence Synthetic polynucleotide
tcctgattgtttctatagttagcgtaacggattttgctaaac

SEQ ID NO: 181 - DNA, 42 bp, Artificial Sequence
  Description of Artificial Sequence Synthetic polynucleotide
tgggattatacttcctcagagccaccacaccctcagaaccgc

SEQ ID NO: 182 - DNA, 42 bp, Artificial Sequence
  Description of Artificial Sequence Synthetic polynucleotide
ccggtaccgagctcgaaacgcaataataagcagatagccgaa

SEQ ID NO: 183 - DNA, 42 bp, Artificial Sequence
  Description of Artificial Sequence Synthetic polynucleotide
taattgcgttgcatttttaagaaaagtaacggaatacccaaa

SEQ ID NO: 184 - DNA, 42 bp, Artificial Sequence
  Description of Artificial Sequence Synthetic polynucleotide
ttgctcactgcccggaacaaagtcagaggaattaactgaaca

SEQ ID NO: 185 - DNA, 42 bp, Artificial Sequence
  Description of Artificial Sequence Synthetic polynucleotide
cttgtttagtatcacatcacgcaaattatttataatcagtga

SEQ ID NO: 186 - DNA, 42 bp, Artificial Sequence
  Description of Artificial Sequence Synthetic polynucleotide
atataaagtaccgatcctgagaagtgttaccgttgtagcaat

SEQ ID NO: 187 - DNA, 42 bp, Artificial Sequence
  Description of Artificial Sequence Synthetic polynucleotide
gggacaaaaggtaaagggcgcgtactatcgcttaatgcgccg

SEQ ID NO: 188 - DNA, 42 bp, Artificial Sequence
  Description of Artificial Sequence Synthetic polynucleotide
ttcaacatgttttatgcctaatgagtgaaacatacgagccgg

SEQ ID NO: 189 - DNA, 42 bp, Artificial Sequence
  Description of Artificial Sequence Synthetic polynucleotide
atattttcatttgctcacaattccacacgctaactcacatta

SEQ ID NO: 190 - DNA, 42 bp, Artificial Sequence
  Description of Artificial Sequence Synthetic polynucleotide
ctggggcgcgagctgccagcacgcgtgcgtcataccgggggt

SEQ ID NO: 191 - DNA, 42 bp, Artificial Sequence
  Description of Artificial Sequence Synthetic polynucleotide
agtggttccgaaatctccagccagctttgaggggacgacgac

SEQ ID NO: 192 - DNA, 42 bp, Artificial Sequence
  Description of Artificial Sequence Synthetic polynucleotide
gcgatggcccacgcgcatctgccagtttccggcaccgcttct

SEQ ID NO: 193 - DNA, 42 bp, Artificial Sequence
  Description of Artificial Sequence Synthetic polynucleotide
aatacgtgaaccatagctttcatcaacatctggccttcctgt

SEQ ID NO: 194 - DNA, 35 bp, Artificial Sequence
  Description of Artificial Sequence Synthetic polynucleotide
caggaggaggctgagactcctacaggagtgtactg

SEQ ID NO: 195 - DNA, 35 bp, Artificial Sequence
  Description of Artificial Sequence Synthetic polynucleotide
tgaatttaccgttccctccctcagagcccaccctc

SEQ ID NO: 196 - DNA, 42 bp, Artificial Sequence
  Description of Artificial Sequence Synthetic polynucleotide
gtaataagttttaaaccctcagagccacgccaccctcagaac

SEQ ID NO: 197 - DNA, 42 bp, Artificial Sequence
  Description of Artificial Sequence Synthetic polynucleotide
atgaaagtattaagttgaggcaggtcaggaaagcgcagtctc

SEQ ID NO: 198 - DNA, 42 bp, Artificial Sequence
  Description of Artificial Sequence Synthetic polynucleotide
cgccagctacaattttaaagccagaatgacgattggccttga

SEQ ID NO: 199 - DNA, 42 bp, Artificial Sequence
  Description of Artificial Sequence Synthetic polynucleotide
gctattttgcaccccggggtcagtgccttattattctgaaac

SEQ ID NO: 200 - DNA, 42 bp, Artificial Sequence
  Description of Artificial Sequence Synthetic polynucleotide
tattcacaaacacccctatttcggaacctgagtaacagtgcc

SEQ ID NO: 201 - DNA, 42 bp, Artificial Sequence
  Description of Artificial Sequence Synthetic polynucleotide
agaataaatcctcattatcctgaatcttaatcaagattagtt

SEQ ID NO: 202 - DNA, 42 bp, Artificial Sequence
  Description of Artificial Sequence Synthetic polynucleotide
cgtataaacagtaagttttgaagccttaaccaacgctaacga

SEQ ID NO: 203 - DNA, 42 bp, Artificial Sequence
  Description of Artificial Sequence Synthetic polynucleotide
actaatgccccctggtttttattttcatatcgagaacaagca

SEQ ID NO: 204 - DNA, 42 bp, Artificial Sequence
  Description of Artificial Sequence Synthetic polynucleotide
gcgtctttccagagcaagtaccgcactccgtaggaatcatta

SEQ ID NO: 205 - DNA, 42 bp, Artificial Sequence
  Description of Artificial Sequence Synthetic polynucleotide
ccgacttgcgggaggaaaaataatatcctagataagtcctga

SEQ ID NO: 206 - DNA, 42 bp, Artificial Sequence
  Description of Artificial Sequence Synthetic polynucleotide
ccgcgcccaatagcctgtttatcaacaacatcctaatttacg

SEQ ID NO: 207 - DNA, 42 bp, Artificial Sequence
  Description of Artificial Sequence Synthetic polynucleotide
aacgggtattaaaccctaatttgccagtttttagcgaacctc

SEQ ID NO: 208 - DNA, 35 bp, Artificial Sequence
  Description of Artificial Sequence Synthetic polynucleotide
agcatgtagaaaccaagaacgcgaggcgtacaaaa

SEQ ID NO: 209 - DNA, 35 bp, Artificial Sequence
  Description of Artificial Sequence Synthetic polynucleotide
aacgcgcaagcaaatcagatacttatcattccaag

SEQ ID NO: 210 - DNA, 35 bp, Artificial Sequence
  Description of Artificial Sequence Synthetic polynucleotide
agaattactcaaatatcaaacccgaacgaaccacc

SEQ ID NO: 211 - DNA, 35 bp, Artificial Sequence
  Description of Artificial Sequence Synthetic polynucleotide
ttaatgcgcgaactatccaaataagaaagcagcct

SEQ ID NO: 212 - DNA, 42 bp, Artificial Sequence
  Description of Artificial Sequence Synthetic polynucleotide
agcagaagataaaacaaaaatgaaaatacgattttttgttta

SEQ ID NO: 213 - DNA, 42 bp, Artificial Sequence
  Description of Artificial Sequence Synthetic polynucleotide
tcaccttgctgaacactgaacaccctgaatggctattagtct

SEQ ID NO: 214 - DNA, 42 bp, Artificial Sequence
  Description of Artificial Sequence Synthetic polynucleotide
acaatacccaaaaggacaatatttttgaacaaagtcagaggg

SEQ ID NO: 215 - DNA, 42 bp, Artificial Sequence
  Description of Artificial Sequence Synthetic polynucleotide
caataataacgggtcagaggtgaggcggaaaaatctaaagca

SEQ ID NO: 216 - DNA, 42 bp, Artificial Sequence
  Description of Artificial Sequence Synthetic polynucleotide
taattgagcgctaagccagcagcaaatgtcagtattaacacc

SEQ ID NO: 217 - DNA, 42 bp, Artificial Sequence
  Description of Artificial Sequence Synthetic polynucleotide
gaatacgtggcacaaactggcatgattaaaaccgaggaaacg

SEQ ID NO: 218 - DNA, 42 bp, Artificial Sequence
  Description of Artificial Sequence Synthetic polynucleotide
gcctgcaacagtataagttaccagaaggagactccttattac

SEQ ID NO: 219 - DNA, 42 bp, Artificial Sequence
  Description of Artificial Sequence Synthetic polynucleotide
acgccacgctgagatatcagagagataaacctgaaagcgtaa

SEQ ID NO: 220 - DNA, 42 bp, Artificial Sequence
  Description of Artificial Sequence Synthetic polynucleotide
gcagtatgttagcaatagaacccttctgcccacaagaattga

SEQ ID NO: 221 - DNA, 42 bp, Artificial Sequence
  Description of Artificial Sequence Synthetic polynucleotide
cagatagccgaacatttgacgctcaatctcatggaaatacct

SEQ ID NO: 222 - DNA, 42 bp, Artificial Sequence
  Description of Artificial Sequence Synthetic polynucleotide
gttaagcccaataaaacaggaaaaacgcgtctgaaatggatt

SEQ ID NO: 223 - DNA, 42 bp, Artificial Sequence
  Description of Artificial Sequence Synthetic polynucleotide
tctggccaacagagaacgtagaaaatacttaagaaaagtaag

SEQ ID NO: 224 - DNA, 35 bp, Artificial Sequence
  Description of Artificial Sequence Synthetic polynucleotide
atttacattggcagtaccgaagccctttatacata

SEQ ID NO: 225 - DNA, 35 bp, Artificial Sequence
  Description of Artificial Sequence Synthetic polynucleotide
ccattgctaagagcaagaaacaataaaagggacat

SEQ ID NO: 226 - DNA, 35 bp, Artificial Sequence
  Description of Artificial Sequence Synthetic polynucleotide
gagggagggtgtgttcagcaatcattgcaggcgct

SEQ ID NO: 227 - DNA, 35 bp, Artificial Sequence
  Description of Artificial Sequence Synthetic polynucleotide
ccacgcaaccagctgcaaagacaccacgattcata

SEQ ID NO: 228 - DNA, 42 bp, Artificial Sequence
  Description of Artificial Sequence Synthetic polynucleotide
ttcgcactcaatccacaatcaatagaaagaataagtttattt

SEQ ID NO: 229 - DNA, 42 bp, Artificial Sequence
  Description of Artificial Sequence Synthetic polynucleotide
acatcccttacactggaaggtaaatatttcggtggtgccatc

SEQ ID NO: 230 - DNA, 42 bp, Artificial Sequence
  Description of Artificial Sequence Synthetic polynucleotide
tgagcgcgttttcacaacggcagcaccggacggaaattattc

SEQ ID NO: 231 - DNA, 42 bp, Artificial Sequence
  Description of Artificial Sequence Synthetic polynucleotide
gcgtcagactgttcgccgggcgcggttgtttgctcgtcataa

SEQ ID NO: 232 - DNA, 42 bp, Artificial Sequence
  Description of Artificial Sequence Synthetic polynucleotide
attaaaggtgaatttgggtaaaggtttccggtatgagccggg

SEQ ID NO: 233 - DNA, 42 bp, Artificial Sequence
  Description of Artificial Sequence Synthetic polynucleotide
aaccgcaagaatgctcggcattttcggtcaagtttgccttta

SEQ ID NO: 234 - DNA, 42 bp, Artificial Sequence
  Description of Artificial Sequence Synthetic polynucleotide
tcactgttgcccgtagtagcgacagaatcatagcccccttat

SEQ ID NO: 235 - DNA, 42 bp, Artificial Sequence
  Description of Artificial Sequence Synthetic polynucleotide
cctgcggctggtaaatcaccgtcaccgagtctggtcagcagc

SEQ ID NO: 236 - DNA, 42 bp, Artificial Sequence
  Description of Artificial Sequence Synthetic polynucleotide
tagcgtttgccatctcagcgtggtgctgcttgagccatttgg

SEQ ID NO: 237 - DNA, 42 bp, Artificial Sequence
  Description of Artificial Sequence Synthetic polynucleotide
gcagcaccgtaatctccggcaaacgcggaacgatgctgattg

SEQ ID NO: 238 - DNA, 42 bp, Artificial Sequence
  Description of Artificial Sequence Synthetic polynucleotide
gaattagagccagcgaagggtaaagttatccgttttttcgtc

SEQ ID NO: 239 - DNA, 42 bp, Artificial Sequence
  Description of Artificial Sequence Synthetic polynucleotide
ggacttgtagaacgttttcataatcaaatgaaaccatcgata

SEQ ID NO: 240 - DNA, 35 bp, Artificial Sequence
  Description of Artificial Sequence Synthetic polynucleotide
tcgtcgctggcagcggaaacgtcaccaaatcaccg

SEQ ID NO: 241 - DNA, 35 bp, Artificial Sequence
  Description of Artificial Sequence Synthetic polynucleotide
tagtgataaaatcaccagtagtaacggaacgtgcc

SEQ ID NO: 242 - DNA, 35 bp, Artificial Sequence
  Description of Artificial Sequence Synthetic polynucleotide
gtcaatctaaaaaaatcccgtgagatagactttct

SEQ ID NO: 243 - DNA, 35 bp, Artificial Sequence
  Description of Artificial Sequence Synthetic polynucleotide
ggcgaaacgtacaggaaacaaagtacaatcgaaat

SEQ ID NO: 244 - DNA, 42 bp, Artificial Sequence
  Description of Artificial Sequence Synthetic polynucleotide
ccgtggtgaagggacctgataaattgtgcggagatttgtatc

SEQ ID NO: 245 - DNA, 42 bp, Artificial Sequence
  Description of Artificial Sequence Synthetic polynucleotide
tgtgtacatcgacaataagggaaccgaacaccggaaacaatc

SEQ ID NO: 246 - DNA, 42 bp, Artificial Sequence
  Description of Artificial Sequence Synthetic polynucleotide
atgagatggtttaaggaacggataacctctgaccaactttga

SEQ ID NO: 247 - DNA, 42 bp, Artificial Sequence
  Description of Artificial Sequence Synthetic polynucleotide
taaattgggcttcgtagctctcacggaaagcagttgggcggt

SEQ ID NO: 248 - DNA, 42 bp, Artificial Sequence
  Description of Artificial Sequence Synthetic polynucleotide
aagaggacagatgagctcatttgccgccaaagagacgcagaa

SEQ ID NO: 249 - DNA, 42 bp, Artificial Sequence
  Description of Artificial Sequence Synthetic polynucleotide
gtggagccgccacgtttcaactttaatcaccagaacgagtag

SEQ ID NO: 250 - DNA, 42 bp, Artificial Sequence
  Description of Artificial Sequence Synthetic polynucleotide
acagcggatcaagtccctgacgagaaacattgtgaattacct

SEQ ID NO: 251 - DNA, 42 bp, Artificial Sequence
  Description of Artificial Sequence Synthetic polynucleotide
atacttaaatttctacggtgtacagaccgcgtaaattcagag

SEQ ID NO: 252 - DNA, 42 bp, Artificial Sequence
  Description of Artificial Sequence Synthetic polynucleotide
tatgcgattttaagcttgacatatcccgaggcgcataggctg

SEQ ID NO: 253 - DNA, 42 bp, Artificial Sequence
  Description of Artificial Sequence Synthetic polynucleotide
gtgaataaggcttggctgcaaggcgattctggcgaaaggggg

SEQ ID NO: 254 - DNA, 42 bp, Artificial Sequence
  Description of Artificial Sequence Synthetic polynucleotide
gctgaccttcatcacgctattacgccagaagttgggtaacgc

SEQ ID NO: 255 - DNA, 42 bp, Artificial Sequence
  Description of Artificial Sequence Synthetic polynucleotide
acttcatgcttcggaactggctcattataagctgctcattca

SEQ ID NO: 256 - DNA, 35 bp, Artificial Sequence
  Description of Artificial Sequence Synthetic polynucleotide
cagggttttcccagaaatcaacgtaacaaccagtc

SEQ ID NO: 257 - DNA, 35 bp, Artificial Sequence
  Description of Artificial Sequence Synthetic polynucleotide
gcctcttagagtaatcttgacccagtgccaagctt

SEQ ID NO: 258 - DNA, 35 bp, Artificial Sequence
  Description of Artificial Sequence Synthetic polynucleotide
gtctggctcaggctgcgcaacgtaaccgtgcatct

SEQ ID NO: 259 - DNA, 35 bp, Artificial Sequence
  Description of Artificial Sequence Synthetic polynucleotide
ttgaccgtaatgggaaattgtaaacgttttaaatc

SEQ ID NO: 260 - DNA, 42 bp, Artificial Sequence
  Description of Artificial Sequence Synthetic polynucleotide
gccagtttgagggggcattaaatttttgaatattttgttaaa

SEQ ID NO: 261 - DNA, 42 bp, Artificial Sequence
  Description of Artificial Sequence Synthetic polynucleotide
gcgccattcgccatcttcctgtagccaggaacaaacggcgga

SEQ ID NO: 262 - DNA, 42 bp, Artificial Sequence
  Description of Artificial Sequence Synthetic polynucleotide
atactcacattaatcggattctccgtggctttcatcaacatt

SEQ ID NO: 263 - DNA, 42 bp, Artificial Sequence
  Description of Artificial Sequence Synthetic polynucleotide
atgagtgagctatcacgacgacagtatcggaaaccaggcaaa

SEQ ID NO: 264 - DNA, 42 bp, Artificial Sequence
  Description of Artificial Sequence Synthetic polynucleotide
aaatgtgagcgacgccgcttctggtgccggcctcaggaagat

SEQ ID NO: 265 - DNA, 42 bp, Artificial Sequence
  Description of Artificial Sequence Synthetic polynucleotide
tcgtaacaacccgttgcgttgcgctcacgcctggggtgccta

SEQ ID NO: 266 - DNA, 42 bp, Artificial Sequence
  Description of Artificial Sequence Synthetic polynucleotide
cgcactccagccaggcataaagtgtaaatgcccgctttccag

SEQ ID NO: 267 - DNA, 42 bp, Artificial Sequence
  Description of Artificial Sequence Synthetic polynucleotide
tcagctttccggcatgagcctcctcacagcctgttcttcgcg

SEQ ID NO: 268 - DNA, 42 bp, Artificial Sequence
  Description of Artificial Sequence Synthetic polynucleotide
tcgggaaacctgtcctgccagcacgcgtgttgaggatccccg

SEQ ID NO: 269 - DNA, 42 bp, Artificial Sequence
  Description of Artificial Sequence Synthetic polynucleotide
catacgagccggaaacgatccagcgcagcggtgccccctgca

SEQ ID NO: 270 - DNA, 42 bp, Artificial Sequence
  Description of Artificial Sequence Synthetic polynucleotide
ggtaccgagctcgagcagccagcggtgctgtcactgcgcgcc

SEQ ID NO: 271 - DNA, 42 bp, Artificial Sequence
  Description of Artificial Sequence Synthetic polynucleotide
cataccgggggtttgtgccagctgcattacaattccacacaa

SEQ ID NO: 272 - DNA, 35 bp, Artificial Sequence
  Description of Artificial Sequence Synthetic polynucleotide
tgtgcactctgtggattgttatccgctcaatgaat

SEQ ID NO: 273 - DNA, 35 bp, Artificial Sequence
  Description of Artificial Sequence Synthetic polynucleotide
gttacctattcgtaatcatgggccgttttcacggt

SEQ ID NO: 274 - DNA, 35 bp, Artificial Sequence
  Description of Artificial Sequence Synthetic polynucleotide
gtttgaacagacgacgacaatacagtagggcttaa

SEQ ID NO: 275 - DNA, 35 bp, Artificial Sequence
  Description of Artificial Sequence Synthetic polynucleotide
catatgcgttatacgtaaatgctgatgcacttttt

SEQ ID NO: 276 - DNA, 42 bp, Artificial Sequence
  Description of Artificial Sequence Synthetic polynucleotide
ttgagaatcgccataagaacgcgagaaaaaatccaatcgcaa

SEQ ID NO: 277 - DNA, 42 bp, Artificial Sequence
  Description of Artificial Sequence Synthetic polynucleotide
aaagtaattctgtcataccgaccgtgtgagcctgtttagtat

SEQ ID NO: 278 - DNA, 35 bp, Artificial Sequence
  Description of Artificial Sequence Synthetic polynucleotide
atttgaggcacgtaaaacagagagcggaattatca

SEQ ID NO: 279 - DNA, 35 bp, Artificial Sequence
  Description of Artificial Sequence Synthetic polynucleotide
ttgagtaacattatttggcaaatcaacaatcttta

SEQ ID NO: 280 - DNA, 42 bp, Artificial Sequence
  Description of Artificial Sequence Synthetic polynucleotide
tcatattcctgattaggttatctaaaatgttgaaaggaattg

SEQ ID NO: 281 - DNA, 42 bp, Artificial Sequence
  Description of Artificial Sequence Synthetic polynucleotide
tatcaaaattatttgatttagaagtattttaattttaaaagt

SEQ ID NO: 282 - DNA, 35 bp, Artificial Sequence
  Description of Artificial Sequence Synthetic polynucleotide
gcggtcctaaatcggaacccttccactattaaaga

SEQ ID NO: 283 - DNA, 35 bp, Artificial Sequence
  Description of Artificial Sequence Synthetic polynucleotide
tagcccgagataggtttgcgtattgggcgggcaac

SEQ ID NO: 284 - DNA, 42 bp, Artificial Sequence
  Description of Artificial Sequence Synthetic polynucleotide
acgtggactccaacttcaccagtgagacgccagggtggtttt

SEQ ID NO: 285 - DNA, 42 bp, Artificial Sequence
  Description of Artificial Sequence Synthetic polynucleotide
gtgccgtaaagcacacgctggtttgcccataaatcaaaagaa

SEQ ID NO: 286 - DNA, 35 bp, Artificial Sequence
  Description of Artificial Sequence Synthetic polynucleotide
ttttgcgtccaataaatcatatagatttagtttga

SEQ ID NO: 287 - DNA, 35 bp, Artificial Sequence
  Description of Artificial Sequence Synthetic polynucleotide
agtttcattccatattttaattcgagctcaggtca

SEQ ID NO: 288 - DNA, 42 bp, Artificial Sequence
  Description of Artificial Sequence Synthetic polynucleotide
ccattagatacattgaagcaaactccaatcaaagcgaaccag

SEQ ID NO: 289 - DNA, 42 bp, Artificial Sequence
  Description of Artificial Sequence Synthetic polynucleotide
tagtagcattaacagatggcttagagctgtacggtgtctgga

SEQ ID NO: 290 - DNA, 35 bp, Artificial Sequence
  Description of Artificial Sequence Synthetic polynucleotide
atgacaaactcatctttgacctacagaggctttga

SEQ ID NO: 291 - DNA, 35 bp, Artificial Sequence
  Description of Artificial Sequence Synthetic polynucleotide
ccctcagcagcgaaaaaaaggctccaaagctttcg

SEQ ID NO: 292 - DNA, 42 bp, Artificial Sequence
  Description of Artificial Sequence Synthetic polynucleotide
ggactaaagactttcggtttatcagcttaggagcctttaatt

SEQ ID NO: 293 - DNA, 42 bp, Artificial Sequence
  Description of Artificial Sequence Synthetic polynucleotide
gaatacactaaaaccaaccatcgcccacttgcgggatcgtca

SEQ ID NO: 294 - DNA, 42 bp, Artificial Sequence
  Description of Artificial Sequence Synthetic polynucleotide
cataggtctgagaggagcaaaagaagattacatttaacaatt

SEQ ID NO: 295 - DNA, 42 bp, Artificial Sequence
  Description of Artificial Sequence Synthetic polynucleotide
tcgctattaattaatacatcgggagaaatacagtaacagtac

SEQ ID NO: 296 - DNA, 42 bp, Artificial Sequence
  Description of Artificial Sequence Synthetic polynucleotide
tcatttgaattaccacgtcagatgaatacaataacggattcg

SEQ ID NO: 297 - DNA, 42 bp, Artificial Sequence
  Description of Artificial Sequence Synthetic polynucleotide
catttcaattacctactacctttttaacgcttctgtaaatcg

SEQ ID NO: 298 - DNA, 35 bp, Artificial Sequence
  Description of Artificial Sequence Synthetic polynucleotide
cctgattgctttgagagtgaataaccttctccggc

SEQ ID NO: 299 - DNA, 35 bp, Artificial Sequence
  Description of Artificial Sequence Synthetic polynucleotide
aggtttattttttaatggaaaagaggcgaattatt

SEQ ID NO: 300 - DNA, 42 bp, Artificial Sequence
  Description of Artificial Sequence Synthetic polynucleotide
ctgagtagaagaacacagggcgcgtactttcctcgttagaat

SEQ ID NO: 301 - DNA, 42 bp, Artificial Sequence
  Description of Artificial Sequence Synthetic polynucleotide
tataatcagtgaggaagaaagcgaaagggtggcgagaaagga

SEQ ID NO: 302 - DNA, 42 bp, Artificial Sequence
  Description of Artificial Sequence Synthetic polynucleotide
cagagcgggagctagaaagccggcgaacagcgggcgctaggg

SEQ ID NO: 303 - DNA, 42 bp, Artificial Sequence
  Description of Artificial Sequence Synthetic polynucleotide
cttaatgcgccgcttcaaactatcggccctgagaagtgtttt

SEQ ID NO: 304 - DNA, 35 bp, Artificial Sequence
  Description of Artificial Sequence Synthetic polynucleotide
cgctggcaagtgtaggtacgccagaatcttgctgg

SEQ ID NO: 305 - DNA, 35 bp, Artificial Sequence
  Description of Artificial Sequence Synthetic polynucleotide
tgacgggaacaggaggccgatccacacccgccgcg

SEQ ID NO: 306 - DNA, 42 bp, Artificial Sequence
  Description of Artificial Sequence Synthetic polynucleotide
taccccggttgatataaatgcaatgcctagaaaggccggaga

SEQ ID NO: 307 - DNA, 42 bp, Artificial Sequence
  Description of Artificial Sequence Synthetic polynucleotide
gatctacaaaggcttgtaccaaaaacatgcataaagctaaat

SEQ ID NO: 308 - DNA, 42 bp, Artificial Sequence
  Description of Artificial Sequence Synthetic polynucleotide
cagtcaaatcaccaaataaagcctcagatatgaccctgtaat

SEQ ID NO: 309 - DNA, 42 bp, Artificial Sequence
  Description of Artificial Sequence Synthetic polynucleotide
accctcatatatttatcagaaaagccccagctatttttgaga

SEQ ID NO: 310 - DNA, 35 bp, Artificial Sequence
  Description of Artificial Sequence Synthetic polynucleotide
acttttgcgggagaatgccggagagggtaaaaaca

SEQ ID NO: 311 - DNA, 35 bp, Artificial Sequence
  Description of Artificial Sequence Synthetic polynucleotide
attaagctcaatatgatattctaaaaatttttaga

SEQ ID NO: 312 - DNA, 42 bp, Artificial Sequence
  Description of Artificial Sequence Synthetic polynucleotide
agcaaagcggattgtatcataaccctcgagaggcttttgcaa

SEQ ID NO: 313 - DNA, 42 bp, Artificial Sequence
  Description of Artificial Sequence Synthetic polynucleotide
tcattgaatcccccaagattcatcagttaacattattacagg

SEQ ID NO: 314 - DNA, 42 bp, Artificial Sequence
  Description of Artificial Sequence Synthetic polynucleotide
aagaagttttgccacgaactaacggaacgagatttaggaata

SEQ ID NO: 315 - DNA, 42 bp, Artificial Sequence
  Description of Artificial Sequence Synthetic polynucleotide
agtaagagcaacaccatcaaaaagattaatcgtcataaatat

SEQ ID NO: 316 - DNA, 35 bp, Artificial Sequence
  Description of Artificial Sequence Synthetic polynucleotide
ccacattcaactaatccaatactgcggaagaggaa

SEQ ID NO: 317 - DNA, 35 bp, Artificial Sequence
  Description of Artificial Sequence Synthetic polynucleotide
aataaaagagggggtaatagtgaattacgaggcat

SEQ ID NO: 318 - DNA, 42 bp, Artificial Sequence
  Description of Artificial Sequence Synthetic polynucleotide
agaaaggaacaactccctcagagccaccgaacccatgtaccg

SEQ ID NO: 319 - DNA, 42 bp, Artificial Sequence
  Description of Artificial Sequence Synthetic polynucleotide
tctaaagttttgtcggttgatataagtagataagtgccgtcg

SEQ ID NO: 320 - DNA, 42 bp, Artificial Sequence
  Description of Artificial Sequence Synthetic polynucleotide
taacactgagtttcctcagtaccaggcgtagcccggaatagg

SEQ ID NO: 321 - DNA, 42 bp, Artificial Sequence
  Description of Artificial Sequence Synthetic polynucleotide
cctcagaaccgccaaaaggaattgcgaaagttagcgtaacga

SEQ ID NO: 322 - DNA, 35 bp, Artificial Sequence
  Description of Artificial Sequence Synthetic polynucleotide
tgtatcaccgtactcagacagccctcattaataat

SEQ ID NO: 323 - DNA, 35 bp, Artificial Sequence
  Description of Artificial Sequence Synthetic polynucleotide
ggttttggtcaccagtacaaactcagaaccgccac

SEQ ID NO: 324 - DNA, 46 bp, Artificial Sequence
  Description of Artificial Sequence Synthetic polynucleotide
ttacagagagaataacttttttttttttttaaaaatacctcaatca

SEQ ID NO: 325 - DNA, 52 bp, Artificial Sequence
  Description of Artificial Sequence Synthetic polynucleotide
taaacagccatatttttttttattctaatcatttttttttcagctaatgcag

SEQ ID NO: 326 - DNA, 46 bp, Artificial Sequence
  Description of Artificial Sequence Synthetic polynucleotide
caaatatattttagttttttttttttttcaacgctcaaaacaacat

SEQ ID NO: 327 - DNA, 52 bp, Artificial Sequence
  Description of Artificial Sequence Synthetic polynucleotide
ttaggttgggttttttttttttatgtatacctttttttttgcgtagattttc

SEQ ID NO: 328 - DNA, 46 bp, Artificial Sequence
  Description of Artificial Sequence Synthetic polynucleotide
ggagcactaacaactattttttttttttcaccagaagaataaagaa

SEQ ID NO: 329 - DNA, 46 bp, Artificial Sequence
  Description of Artificial Sequence Synthetic polynucleotide
agctgattgcccttcattttttttttttgaacaagagaaagggagc

SEQ ID NO: 330 - DNA, 52 bp, Artificial Sequence
  Description of Artificial Sequence Synthetic polynucleotide
cggccaacgcgctttttttttgtgaatgctgtttttttttatcagatgccgg

SEQ ID NO: 331 - DNA, 46 bp, Artificial Sequence
  Description of Artificial Sequence Synthetic polynucleotide
tggtttaccagcgccatttttttttttttcagcggggatcgttaac

SEQ ID NO: 332 - DNA, 52 bp, Artificial Sequence
  Description of Artificial Sequence Synthetic polynucleotide
aaggtggcaacattttttttttatctattcattttttttttattaccgccag

SEQ ID NO: 333 - DNA, 52 bp, Artificial Sequence
  Description of Artificial Sequence Synthetic polynucleotide
taatatccagaatttttttttggaacgcggttttttttttcgatttagagct

SEQ ID NO: 334 - DNA, 46 bp, Artificial Sequence
  Description of Artificial Sequence Synthetic polynucleotide
aggtgaatttcttaaattttttttttttagcaacggccccagcgat

SEQ ID NO: 335 - DNA, 52 bp, Artificial Sequence
  Description of Artificial Sequence Synthetic polynucleotide
tttttcacgttgtttttttttttccacaggattttttttttaggattagcgg

SEQ ID NO: 336 - DNA, 46 bp, Artificial Sequence
  Description of Artificial Sequence Synthetic polynucleotide
agagccgccaccagaatttttttttttttttgatgatcaagagaag

SEQ ID NO: 337 - DNA, 52 bp, Artificial Sequence
  Description of Artificial Sequence Synthetic polynucleotide
gaaccagagccatttttttttaggccctccgtttttttttacaggcggcctt

SEQ ID NO: 338 - DNA, 46 bp, Artificial Sequence
  Description of Artificial Sequence Synthetic polynucleotide
ccgcgacctgctccatttttttttttttaatttgtgaaaaaaaagc

SEQ ID NO: 339 - DNA, 46 bp, Artificial Sequence
  Description of Artificial Sequence Synthetic polynucleotide
agctcattttttaaccttttttttttttgggcgcatctgttgggaa

SEQ ID NO: 340 - DNA, 52 bp, Artificial Sequence
  Description of Artificial Sequence Synthetic polynucleotide
ggaagattgtattttttttttaattaagccttttttttttagaattagcaaa

SEQ ID NO: 341 - DNA, 46 bp, Artificial Sequence
  Description of Artificial Sequence Synthetic polynucleotide
ggattagagagtacctttttttttttttgcgaacgagcaggcaagg

SEQ ID NO: 342 - DNA, 52 bp, Artificial Sequence
  Description of Artificial Sequence Synthetic polynucleotide
gcccgaaagactttttttttttagcgtgcagtttttttttaaaatctacgtt

SEQ ID NO: 343 - DNA, 52 bp, Artificial Sequence
  Description of Artificial Sequence Synthetic polynucleotide
aggacgttgggattttttttttaccctcacgtttttttttcgatcggtgcgg

SEQ ID NO: 344 - DNA, 52 bp, Artificial Sequence
  Description of Artificial Sequence Synthetic polynucleotide
tttatcccagatagccctttttctttctagaatttttgacctaaatttaatg

SEQ ID NO: 345 - DNA, 52 bp, Artificial Sequence
  Description of Artificial Sequence Synthetic polynucleotide
taactatataaattcttattttcgcgccagtattttcgtcaatagataatac

SEQ ID NO: 346 - DNA, 38 bp, Artificial Sequence
  Description of Artificial Sequence Synthetic polynucleotide
tctggtcagcattttgcgttttaagcgcattagacggg

SEQ ID NO: 347 - DNA, 52 bp, Artificial Sequence
  Description of Artificial Sequence Synthetic polynucleotide
gagaggcgggttgagtgtttttggcggtcatattttcgacattcaaccgatt

SEQ ID NO: 348 - DNA, 32 bp, Artificial Sequence
  Description of Artificial Sequence Synthetic polynucleotide
aaaagaaactacggctggttttccagtaatga

SEQ ID NO: 349 - DNA, 30 bp, Artificial Sequence
  Description of Artificial Sequence Synthetic polynucleotide
aaccataaagttttgagagagttgcagcaa

SEQ ID NO: 350 - DNA, 52 bp, Artificial Sequence
  Description of Artificial Sequence Synthetic polynucleotide
atctccaaaagacagcatttttccaccctacattttccgccgccagcattga

SEQ ID NO: 351 - DNA, 52 bp, Artificial Sequence
  Description of Artificial Sequence Synthetic polynucleotide
ccggaaccgcagtaagcgttttcctcacaccattttgaacgaggcgcagacg

SEQ ID NO: 352 - DNA, 38 bp, Artificial Sequence
  Description of Artificial Sequence Synthetic polynucleotide
accaagcgccgccatgttttttgatagttgcgccgaca

SEQ ID NO: 353 - DNA, 52 bp, Artificial Sequence
  Description of Artificial Sequence Synthetic polynucleotide
caaatatttataggtcacttttaaggaaaccgtttttttgataagaggtcat

SEQ ID NO: 354 - DNA, 32 bp, Artificial Sequence
  Description of Artificial Sequence Synthetic polynucleotide
aatatcgcgtaacagttgttttaaaagaaaat

SEQ ID NO: 355 - DNA, 30 bp, Artificial Sequence
  Description of Artificial Sequence Synthetic polynucleotide
gacggaagaattttatcaaaaataattcgc

SEQ ID NO: 356 - DNA, 42 bp, Artificial Sequence
  Description of Artificial Sequence Synthetic polynucleotide
gacaatcatatgtataattactagaaaaataaataaggcgtt

SEQ ID NO: 357 - DNA, 42 bp, Artificial Sequence
  Description of Artificial Sequence Synthetic polynucleotide
aaactagcatgtcaatttaacaacgccataccgacaaaaggt

SEQ ID NO: 358 - DNA, 42 bp, Artificial Sequence
  Description of Artificial Sequence Synthetic polynucleotide
aaataagaataacaaagagaatataaagacatgtaatttagg

SEQ ID NO: 359 - DNA, 42 bp, Artificial Sequence
  Description of Artificial Sequence Synthetic polynucleotide
agacgttagtaaatttgcccgaacgttaagactttacaaaca

SEQ ID NO: 360 - DNA, 42 bp, Artificial Sequence
  Description of Artificial Sequence Synthetic polynucleotide
tcgtctttccaggaatcagatgatggcagttagaacctacca

SEQ ID NO: 361 - DNA, 42 bp, Artificial Sequence
  Description of Artificial Sequence Synthetic polynucleotide
attcgacaactccctgaataatggaaggattcatcaatataa

SEQ ID NO: 362 - DNA, 42 bp, Artificial Sequence
  Description of Artificial Sequence Synthetic polynucleotide
tcaggaagatcgcacggcaaaatcccttcagcaggcgaaaat

SEQ ID NO: 363 - DNA, 42 bp, Artificial Sequence
  Description of Artificial Sequence Synthetic polynucleotide
agtatcggcctcttgtcaaagggcgaaatttttggggtcgag

SEQ ID NO: 364 - DNA, 42 bp, Artificial Sequence
  Description of Artificial Sequence Synthetic polynucleotide
cctgtttgatggcccacccaaatcaagtaaccgtctatcagg

SEQ ID NO: 365 - DNA, 42 bp, Artificial Sequence
  Description of Artificial Sequence Synthetic polynucleotide
actaaagcctggggaatatgcaactaaataattgctgaatat

SEQ ID NO: 366 - DNA, 42 bp, Artificial Sequence
  Description of Artificial Sequence Synthetic polynucleotide
aagcataaagtgcgtcgcaaatggtcaaaattctactaatag

SEQ ID NO: 367 - DNA, 42 bp, Artificial Sequence
  Description of Artificial Sequence Synthetic polynucleotide
aatgctgtagctctgaaaaggtggcatctaacctgtttagct

SEQ ID NO: 368 - DNA, 42 bp, Artificial Sequence
  Description of Artificial Sequence Synthetic polynucleotide
gtatgaaagtattagttaaaggccgcttgcataaccgatata

SEQ ID NO: 369 - DNA, 42 bp, Artificial Sequence
  Description of Artificial Sequence Synthetic polynucleotide
ttattctgaaacatttcatgaggaagttcgaaagaggcaaaa

SEQ ID NO: 370 - DNA, 42 bp, Artificial Sequence
  Description of Artificial Sequence Synthetic polynucleotide
ttcggtcgctgagcgcaccaacctaaaatccattaaacgggt

SEQ ID NO: 371 - DNA, 42 bp, Artificial Sequence
  Description of Artificial Sequence Synthetic polynucleotide
caggaaacaatcgggaagagtcaatagtccttgaaaacatag

SEQ ID NO: 372 - DNA, 42 bp, Artificial Sequence
  Description of Artificial Sequence Synthetic polynucleotide
ttgccgccagcattttttcccttagaatgaatttatcaaaat

SEQ ID NO: 373 - DNA, 42 bp, Artificial Sequence
  Description of Artificial Sequence Synthetic polynucleotide
ctgttgggcggttgaaaacaaaattaatgatgaaacaaacat

SEQ ID NO: 374 - DNA, 42 bp, Artificial Sequence
  Description of Artificial Sequence Synthetic polynucleotide
gaccacgcataaccttctttgattagtaagtctgtccatcac

SEQ ID NO: 375 - DNA, 42 bp, Artificial Sequence
  Description of Artificial Sequence Synthetic polynucleotide
ttttcatgaggaggccaccgagtaaaagataacatcacttgc

SEQ ID NO: 376 - DNA, 42 bp, Artificial Sequence
  Description of Artificial Sequence Synthetic polynucleotide
agagtttccattaaacgtataacgtgctatggttgctttgac

SEQ ID NO: 377 - DNA, 42 bp, Artificial Sequence
  Description of Artificial Sequence Synthetic polynucleotide
tacttgacatatccatcgtaaaactagctgagagtctggagc

SEQ ID NO: 378 - DNA, 42 bp, Artificial Sequence
  Description of Artificial Sequence Synthetic polynucleotide
gatagctctcacgtatcaggtcattgccatgtcaatcatatg

SEQ ID NO: 379 - DNA, 42 bp, Artificial Sequence
  Description of Artificial Sequence Synthetic polynucleotide
cgggaaaaagagacgattcaaaagggtggagtaatgtgtagg

SEQ ID NO: 380 - DNA, 42 bp, Artificial Sequence
  Description of Artificial Sequence Synthetic polynucleotide
atgaattagagccaggtctttaccctgacagttcagaaaacg

SEQ ID NO: 381 - DNA, 42 bp, Artificial Sequence
  Description of Artificial Sequence Synthetic polynucleotide
ttcatcggcattgatcaaatgctttaaactattatagtcaga

SEQ ID NO: 382 - DNA, 42 bp, Artificial Sequence
  Description of Artificial Sequence Synthetic polynucleotide
tattcggtcatagcaaaccaaaatagcgtttaccagacgacg

SEQ ID NO: 383 - DNA, 42 bp, Artificial Sequence
  Description of Artificial Sequence Synthetic polynucleotide
ggggtgtctggaagctttcaacagtttctagtaaatgaattt

SEQ ID NO: 384 - DNA, 42 bp, Artificial Sequence
  Description of Artificial Sequence Synthetic polynucleotide
gtggcatcaattaggtctttccagacgtagcggagtgagaat

SEQ ID NO: 385 - DNA, 42 bp, Artificial Sequence
  Description of Artificial Sequence Synthetic polynucleotide
agctactaatagtaagcaagcccaatagaccctcattttcag

SEQ ID NO: 386 - DNA, 35 bp, Artificial Sequence
  Description of Artificial Sequence Synthetic polynucleotide
aaacaatagattttcaggtttcagatgatggcaat

SEQ ID NO: 387 - DNA, 35 bp, Artificial Sequence
  Description of Artificial Sequence Synthetic polynucleotide
gaaaccaccagaaggaaggttatctaaaagagccg

SEQ ID NO: 388 - DNA, 42 bp, Artificial Sequence
  Description of Artificial Sequence Synthetic polynucleotide
tcatcaatataatcacaactaatagattatatctttaggagc

SEQ ID NO: 389 - DNA, 42 bp, Artificial Sequence
  Description of Artificial Sequence Synthetic polynucleotide
taaagaaattgcgttcgacaactcgtatttttgcggaacaaa

SEQ ID NO: 390 - DNA, 42 bp, Artificial Sequence
  Description of Artificial Sequence Synthetic polynucleotide
acgtgccgtaaagcgagtaacattatcataaatcctttgccc

SEQ ID NO: 391 - DNA, 42 bp, Artificial Sequence
  Description of Artificial Sequence Synthetic polynucleotide
tttggggtcgagtactgattgtttggatacgtaaaacagaaa

SEQ ID NO: 392 - DNA, 42 bp, Artificial Sequence
  Description of Artificial Sequence Synthetic polynucleotide
gaacgttattaaattcaaaattatttgctatacttctgaata

SEQ ID NO: 393 - DNA, 42 bp, Artificial Sequence
  Description of Artificial Sequence Synthetic polynucleotide
aattttaaaagtttactaaatcggaacccccaaatcaagttt

SEQ ID NO: 394 - DNA, 42 bp, Artificial Sequence
  Description of Artificial Sequence Synthetic polynucleotide
atggaagggttaaatacgtgaaccatcactaaagggagcccc

SEQ ID NO: 395 - DNA, 42 bp, Artificial Sequence
  Description of Artificial Sequence Synthetic polynucleotide
gcgaacctaccatacaaaagaatagcccgcaaaatcccttat

SEQ ID NO: 396 - DNA, 42 bp, Artificial Sequence
  Description of Artificial Sequence Synthetic polynucleotide
cgatttagagcttgtggttccgaaatcggagatagggttgag

SEQ ID NO: 397 - DNA, 42 bp, Artificial Sequence
  Description of Artificial Sequence Synthetic polynucleotide
gggcgatggcccaccagctgattgccctcaccagtgagacgg

SEQ ID NO: 398 - DNA, 42 bp, Artificial Sequence
  Description of Artificial Sequence Synthetic polynucleotide
tgttgttccagtttgtggtttttctttttcaccgcctggccc

SEQ ID NO: 399 - DNA, 42 bp, Artificial Sequence
  Description of Artificial Sequence Synthetic polynucleotide
atcctgtttgatggacggggaaagccggaaaaccgtctatca

SEQ ID NO: 400 - DNA, 35 bp, Artificial Sequence
  Description of Artificial Sequence Synthetic polynucleotide
tgagagagttgcagacgtcaaagggcgacgaacgt

SEQ ID NO: 401 - DNA, 35 bp, Artificial Sequence
  Description of Artificial Sequence Synthetic polynucleotide
cgccaggggaacaagagtccacccagcaggcgaaa

SEQ ID NO: 402 - DNA, 35 bp, Artificial Sequence
  Description of Artificial Sequence Synthetic polynucleotide
tatggttttacgccagctggcgaagatcgcactcc

SEQ ID NO: 403 - DNA, 35 bp, Artificial Sequence
  Description of Artificial Sequence Synthetic polynucleotide
gtgcatctgccagtgaaaggagcgggcgcgcgtaa

SEQ ID NO: 404 - DNA, 42 bp, Artificial Sequence
  Description of Artificial Sequence Synthetic polynucleotide
agccagctttccggtagcggtcacgctgctagggcgctggca

SEQ ID NO: 405 - DNA, 42 bp, Artificial Sequence
  Description of Artificial Sequence Synthetic polynucleotide
gggcctcttcgctagctttgacgagcacggcgcatcgtaacc

SEQ ID NO: 406 - DNA, 42 bp, Artificial Sequence
  Description of Artificial Sequence Synthetic polynucleotide
agcatcacttgcctcgttggtgtagatggtataacgtgcttt

SEQ ID NO: 407 - DNA, 42 bp, Artificial Sequence
  Description of Artificial Sequence Synthetic polynucleotide
gattagtaataatgcaccgcttctggtgagggcgatcggtgc

SEQ ID NO: 408 - DNA, 42 bp, Artificial Sequence
  Description of Artificial Sequence Synthetic polynucleotide
cctcgttagaatcacgcaactgttgggaccggaaaccaggca

SEQ ID NO: 409 - DNA, 42 bp, Artificial Sequence
  Description of Artificial Sequence Synthetic polynucleotide
aatgggataggtcagagtagaagaactcagcaatacttcttt

SEQ ID NO: 410 - DNA, 42 bp, Artificial Sequence
  Description of Artificial Sequence Synthetic polynucleotide
aagcgccattcgataaattaaccgttgtaaactatcggcctt

SEQ ID NO: 411 - DNA, 42 bp, Artificial Sequence
  Description of Artificial Sequence Synthetic polynucleotide
caccattcaggctggagcgggagctaaaggcggattgaccgt

SEQ ID NO: 412 - DNA, 42 bp, Artificial Sequence
  Description of Artificial Sequence Synthetic polynucleotide
gctggtaatatccaccgtgggaacaaaccaggaggccgatta

SEQ ID NO: 413 - DNA, 42 bp, Artificial Sequence
  Description of Artificial Sequence Synthetic polynucleotide
tctgtccatcacgcaggaacgccatcaagctcattttttaac

SEQ ID NO: 414 - DNA, 42 bp, Artificial Sequence
  Description of Artificial Sequence Synthetic polynucleotide
aagggattttagactttttgttaaatcaaaataattcgcgtc

SEQ ID NO: 415 - DNA, 42 bp, Artificial Sequence
  Description of Artificial Sequence Synthetic polynucleotide
acccgtcggattctgaacaatattaccgaccgagtaaaagag

SEQ ID NO: 416 - DNA, 35 bp, Artificial Sequence
  Description of Artificial Sequence Synthetic polynucleotide
tggccttcctgtagtaatcagtgaggccccagcca

SEQ ID NO: 417 - DNA, 35 bp, Artificial Sequence
  Description of Artificial Sequence Synthetic polynucleotide
cattaaaaggaacggtacgccgtgagcgagtaaca

SEQ ID NO: 418 - DNA, 35 bp, Artificial Sequence
  Description of Artificial Sequence Synthetic polynucleotide
aacagagtctacgttaataaaacaccagaacgagt

SEQ ID NO: 419 - DNA, 35 bp, Artificial Sequence
  Description of Artificial Sequence Synthetic polynucleotide
aacaaagctgctcaaaatacctacatttattggca

SEQ ID NO: 420 - DNA, 42 bp, Artificial Sequence
  Description of Artificial Sequence Synthetic polynucleotide
agtaaattgggcttaatggattatttactgacgctcaatcgt

SEQ ID NO: 421 - DNA, 42 bp, Artificial Sequence
  Description of Artificial Sequence Synthetic polynucleotide
gttgggaagaaaaaatagaacccttctgacccaaatcaacgt

SEQ ID NO: 422 - DNA, 42 bp, Artificial Sequence
  Description of Artificial Sequence Synthetic polynucleotide
ctcaaatgaaaaataccggatattcattacctgaaagcgtaa

SEQ ID NO: 423 - DNA, 42 bp, Artificial Sequence
  Description of Artificial Sequence Synthetic polynucleotide
gagagccagcaggagagatggtttaattataccagtcaggac

SEQ ID NO: 424 - DNA, 42 bp, Artificial Sequence
  Description of Artificial Sequence Synthetic polynucleotide
gaatacgtggcacaagaactggctcatttcaactttaatcat

SEQ ID NO: 425 - DNA, 42 bp, Artificial Sequence
  Description of Artificial Sequence Synthetic polynucleotide
taatcttgacaagactaaagcatcacctaacagtgccacgct

SEQ ID NO: 426 - DNA, 42 bp, Artificial Sequence
  Description of Artificial Sequence Synthetic polynucleotide
tgtgaattacctttttaacaccgcctgctgctgaacctcaaa

SEQ ID NO: 427 - DNA, 42 bp, Artificial Sequence
  Description of Artificial Sequence Synthetic polynucleotide
tgtatgcgattttagacaatatttttgaaccttcatcaagag

SEQ ID NO: 428 - DNA, 42 bp, Artificial Sequence
  Description of Artificial Sequence Synthetic polynucleotide
tatcaaaccctcaagcataggctggctgatggctattagtct

SEQ ID NO: 429 - DNA, 42 bp, Artificial Sequence
  Description of Artificial Sequence Synthetic polynucleotide
tgaggcggtcagtaacttagccggaacgcgcgacctgctcca

SEQ ID NO: 430 - DNA, 42 bp, Artificial Sequence
  Description of Artificial Sequence Synthetic polynucleotide
ttaatgcgcgaactattgtgtcgaaatcaggcgcagacggtc

SEQ ID NO: 431 - DNA, 42 bp, Artificial Sequence
  Description of Artificial Sequence Synthetic polynucleotide
tgtacagaccaggctcaatatctggtcaagataaaacagagg

SEQ ID NO: 432 - DNA, 35 bp, Artificial Sequence
  Description of Artificial Sequence Synthetic polynucleotide
aatcataagggaacgaaccaccagcagagttggca

SEQ ID NO: 433 - DNA, 35 bp, Artificial Sequence
  Description of Artificial Sequence Synthetic polynucleotide
ctgataagatagccctaaaacggacagatgaacgg

SEQ ID NO: 434 - DNA, 35 bp, Artificial Sequence
  Description of Artificial Sequence Synthetic polynucleotide
attaaacagcgcgaaacaaagcatgaggaagtttc

SEQ ID NO: 435 - DNA, 35 bp, Artificial Sequence
  Description of Artificial Sequence Synthetic polynucleotide
agggtagcaacggcagtcctgaacaagatagaaac

SEQ ID NO: 436 - DNA, 42 bp, Artificial Sequence
  Description of Artificial Sequence Synthetic polynucleotide
cattaaacgggtaaaatttacgagcatgaaaataatatccca

SEQ ID NO: 437 - DNA, 42 bp, Artificial Sequence
  Description of Artificial Sequence Synthetic polynucleotide
cagcgattataccacaagtaccgcactcacagcatcggaacg

SEQ ID NO: 438 - DNA, 42 bp, Artificial Sequence
  Description of Artificial Sequence Synthetic polynucleotide
tcttttatcctgaactcagcagcgaaagatcgagaacaagca

SEQ ID NO: 439 - DNA, 42 bp, Artificial Sequence
  Description of Artificial Sequence Synthetic polynucleotide
acccagctacaactaatacgtaatgccatcatctttgacccc

SEQ ID NO: 440 - DNA, 42 bp, Artificial Sequence
  Description of Artificial Sequence Synthetic polynucleotide
agccgtttttatttatacactaaaacacctacgaaggcacca

SEQ ID NO: 441 - DNA, 42 bp, Artificial Sequence
  Description of Artificial Sequence Synthetic polynucleotide
gcgggatcgtcacctcttaccaacgctaagttgctattttgc

SEQ ID NO: 442 - DNA, 42 bp, Artificial Sequence
  Description of Artificial Sequence Synthetic polynucleotide
acctaaaacgaatccttaaatcaagattacgagcgtctttcc

SEQ ID NO: 443 - DNA, 42 bp, Artificial Sequence
  Description of Artificial Sequence Synthetic polynucleotide
ttagaggcaaaagatcatcgtaggaatctaaaggccgctttt

SEQ ID NO: 444 - DNA, 42 bp, Artificial Sequence
  Description of Artificial Sequence Synthetic polynucleotide
agagcctaatttgcggcttgcagggagtattaccgcgcccaa

SEQ ID NO: 445 - DNA, 42 bp, Artificial Sequence
  Description of Artificial Sequence Synthetic polynucleotide
ggaggttttgaagcgaggtgaatttcttgtttatcagcttgc

SEQ ID NO: 446 - DNA, 42 bp, Artificial Sequence
  Description of Artificial Sequence Synthetic polynucleotide
tagcaagcaaatcactttaattgtatcgaaacagcttgatac

SEQ ID NO: 447 - DNA, 42 bp, Artificial Sequence
  Description of Artificial Sequence Synthetic polynucleotide
tattcggtcgctgacagttacaaaataacctcccgacttgcg

SEQ ID NO: 448 - DNA, 35 bp, Artificial Sequence
  Description of Artificial Sequence Synthetic polynucleotide
cgatagttgcgccgggcgttttagcgaaacagcca

SEQ ID NO: 449 - DNA, 35 bp, Artificial Sequence
  Description of Artificial Sequence Synthetic polynucleotide
aaggagcgatatagaaggcttacgcataaccgata

SEQ ID NO: 450 - DNA, 35 bp, Artificial Sequence
  Description of Artificial Sequence Synthetic polynucleotide
caccctcttttttcacgttgagttttgtcgtcttt

SEQ ID NO: 451 - DNA, 35 bp, Artificial Sequence
  Description of Artificial Sequence Synthetic polynucleotide
tagcattccacagataagtatagcccggtaccgcc

SEQ ID NO: 452 - DNA, 42 bp, Artificial Sequence
  Description of Artificial Sequence Synthetic polynucleotide
ccagacgttagtaactcaggaggtttagaataggtgtatcac

SEQ ID NO: 453 - DNA, 42 bp, Artificial Sequence
  Description of Artificial Sequence Synthetic polynucleotide
attgcgaataataaattttcagggatagaactacaacgcctg

SEQ ID NO: 454 - DNA, 42 bp, Artificial Sequence
  Description of Artificial Sequence Synthetic polynucleotide
cgtatagtcagaagtcgtcaccagtacacaagcccaatagga

SEQ ID NO: 455 - DNA, 42 bp, Artificial Sequence
  Description of Artificial Sequence Synthetic polynucleotide
taccctgactattaatgaattttctgtagaacaactaaagga

SEQ ID NO: 456 - DNA, 42 bp, Artificial Sequence
  Description of Artificial Sequence Synthetic polynucleotide
acccatgtaccgaagtgagaatagaaagtgggattttgctaa

SEQ ID NO: 457 - DNA, 42 bp, Artificial Sequence
  Description of Artificial Sequence Synthetic polynucleotide
aataacactgagttcaaagcggattgcaaaaaatcaggtctt

SEQ ID NO: 458 - DNA, 42 bp, Artificial Sequence
  Description of Artificial Sequence Synthetic polynucleotide
acaactttcaacccaatgaccataaatctcaaaaagattaag

SEQ ID NO: 459 - DNA, 42 bp, Artificial Sequence
  Description of Artificial Sequence Synthetic polynucleotide
taagtttcagcggagaagttttgccagacgagaggcttttgc

SEQ ID NO: 460 - DNA, 42 bp, Artificial Sequence
  Description of Artificial Sequence Synthetic polynucleotide
aggaagcccgaaagaaaaaccaaaataggggggtaatagtaa

SEQ ID NO: 461 - DNA, 42 bp, Artificial Sequence
  Description of Artificial Sequence Synthetic polynucleotide
gttcagaaaacgagacattcaactaatgttgagatttaggaa

SEQ ID NO: 462 - DNA, 42 bp, Artificial Sequence
  Description of Artificial Sequence Synthetic polynucleotide
aatgtttagactgggaaagattcatcagcagatacataacgc

SEQ ID NO: 463 - DNA, 42 bp, Artificial Sequence
  Description of Artificial Sequence Synthetic polynucleotide
taccagacgacgatacttcaaatatcgcaaatgctttaaaca

SEQ ID NO: 464 - DNA, 35 bp, Artificial Sequence
  Description of Artificial Sequence Synthetic polynucleotide
caaaaggaattacgattgaatccccctcgttttaa

SEQ ID NO: 465 - DNA, 35 bp, Artificial Sequence
  Description of Artificial Sequence Synthetic polynucleotide
acaggtaatagcgtccaatactcataaccctcgtt

SEQ ID NO: 466 - DNA, 35 bp, Artificial Sequence
  Description of Artificial Sequence Synthetic polynucleotide
tgcctgttcggccaacgcgcgagccggaagcataa

SEQ ID NO: 467 - DNA, 35 bp, Artificial Sequence
  Description of Artificial Sequence Synthetic polynucleotide
cctgtgtgaaattggcgcagtgtcactgaatgcgg

SEQ ID NO: 468 - DNA, 42 bp, Artificial Sequence
  Description of Artificial Sequence Synthetic polynucleotide
agtgtaaagcctggggtgctgcggccagcgcgcctgtgcact

SEQ ID NO: 469 - DNA, 42 bp, Artificial Sequence
  Description of Artificial Sequence Synthetic polynucleotide
gctgcattaatgaatcttcgcgtccgtgggtcatagctgttt

SEQ ID NO: 470 - DNA, 35 bp, Artificial Sequence
  Description of Artificial Sequence Synthetic polynucleotide
ggcgtaagcggcctttagtgagctctcacggaaaa

SEQ ID NO: 471 - DNA, 35 bp, Artificial Sequence
  Description of Artificial Sequence Synthetic polynucleotide
cccggaatttgtgagcgattaagttgggaaaacga

SEQ ID NO: 472 - DNA, 42 bp, Artificial Sequence
  Description of Artificial Sequence Synthetic polynucleotide
agagacgcagaaacagtcacgacgttgttaacgccagggttt

SEQ ID NO: 473 - DNA, 42 bp, Artificial Sequence
  Description of Artificial Sequence Synthetic polynucleotide
aaaaagccgcacagattcagaggtggagccatgtttaccagt

SEQ ID NO: 474 - DNA, 35 bp, Artificial Sequence
  Description of Artificial Sequence Synthetic polynucleotide
aatataattagcaaaattaaggcaaatggtcaata

SEQ ID NO: 475 - DNA, 35 bp, Artificial Sequence
  Description of Artificial Sequence Synthetic polynucleotide
attctgcgaacgagcggaagcaaactccctccttt

SEQ ID NO: 476 - DNA, 42 bp, Artificial Sequence
  Description of Artificial Sequence Synthetic polynucleotide
acctgtttagctatagtacctttaattgaacaggtcaggatt

SEQ ID NO: 477 - DNA, 42 bp, Artificial Sequence
  Description of Artificial Sequence Synthetic polynucleotide
ggcaaggcaaagaatgctgtagctcaacacagttgattccca

SEQ ID NO: 478 - DNA, 35 bp, Artificial Sequence
  Description of Artificial Sequence Synthetic polynucleotide
tattcatggaaccagagccacccgtaatcagtagc

SEQ ID NO: 479 - DNA, 35 bp, Artificial Sequence
  Description of Artificial Sequence Synthetic polynucleotide
tagcaaggccggaatcacaatcaatagaaagggcg

SEQ ID NO: 480 - DNA, 42 bp, Artificial Sequence
  Description of Artificial Sequence Synthetic polynucleotide
gacagaatcaagttagcgccaaagacaaaaattcatatggtt

SEQ ID NO: 481 - DNA, 42 bp, Artificial Sequence
  Description of Artificial Sequence Synthetic polynucleotide
taatcaaaatcacctaaaggtgaattatagcaccattaccat

SEQ ID NO: 482 - DNA, 35 bp, Artificial Sequence
  Description of Artificial Sequence Synthetic polynucleotide
gcgttaataatgcagaacgcgttaacaacgccaac

SEQ ID NO: 483 - DNA, 35 bp, Artificial Sequence
  Description of Artificial Sequence Synthetic polynucleotide
aaagccaacgctcacaaagaacgcgagatcttctg

SEQ ID NO: 484 - DNA, 42 bp, Artificial Sequence
  Description of Artificial Sequence Synthetic polynucleotide
atgtaatttaggcatttagttaatttcaaaactttttcaaat

SEQ ID NO: 485 - DNA, 42 bp, Artificial Sequence
  Description of Artificial Sequence Synthetic polynucleotide
acaacatgttcagcataagaataaacacattcttaccagtat

SEQ ID NO: 486 - DNA, 42 bp, Artificial Sequence
  Description of Artificial Sequence Synthetic polynucleotide
atcagatgccgggtccgcaagaatgccaaaccagcttacggc

SEQ ID NO: 487 - DNA, 42 bp, Artificial Sequence
  Description of Artificial Sequence Synthetic polynucleotide
ggtcactgttgcccgtcgctggcagcctggtccgttttttcg

SEQ ID NO: 488 - DNA, 42 bp, Artificial Sequence
  Description of Artificial Sequence Synthetic polynucleotide
tggaggtgtccagcgttccggcaaacgcccggccagagcaca

SEQ ID NO: 489 - DNA, 42 bp, Artificial Sequence
  Description of Artificial Sequence Synthetic polynucleotide
ctggtcagcagcaatacctgcagccagctgcggtatgagccg

SEQ ID NO: 490 - DNA, 35 bp, Artificial Sequence
  Description of Artificial Sequence Synthetic polynucleotide
tcctcataacggaaccgccgggcgcggtggtgccg

SEQ ID NO: 491 - DNA, 35 bp, Artificial Sequence
  Description of Artificial Sequence Synthetic polynucleotide
gattgccatcagcggggtcatagcgtggtgctggt

SEQ ID NO: 492 - DNA, 42 bp, Artificial Sequence
  Description of Artificial Sequence Synthetic polynucleotide
aaacaggaagattgaagattcaaaagggaatatgatattcaa

SEQ ID NO: 493 - DNA, 42 bp, Artificial Sequence
  Description of Artificial Sequence Synthetic polynucleotide
agagtctggagcaattttgcgggagaagattatgaccctgta

SEQ ID NO: 494 - DNA, 42 bp, Artificial Sequence
  Description of Artificial Sequence Synthetic polynucleotide
ccgttctagctgatgttgtaccaaaaaccctttatttcaacg

SEQ ID NO: 495 - DNA, 42 bp, Artificial Sequence
  Description of Artificial Sequence Synthetic polynucleotide
gtaatgtgtaggtatataagcaaatattcaggtcattgcctg

SEQ ID NO: 496 - DNA, 35 bp, Artificial Sequence
  Description of Artificial Sequence Synthetic polynucleotide
caaggataaaaatttctacaaaggctattaaattg

SEQ ID NO: 497 - DNA, 35 bp, Artificial Sequence
  Description of Artificial Sequence Synthetic polynucleotide
taaatcgaaattaatgccggaaatgcaatgcctga

SEQ ID NO: 498 - DNA, 42 bp, Artificial Sequence
  Description of Artificial Sequence Synthetic polynucleotide
taggattagcggggaataaatcctcatttaccgttccagtaa

SEQ ID NO: 499 - DNA, 42 bp, Artificial Sequence
  Description of Artificial Sequence Synthetic polynucleotide
cccgtataaacagtcagagccgccaccacctcagagccacca

SEQ ID NO: 500 - DNA, 42 bp, Artificial Sequence
  Description of Artificial Sequence Synthetic polynucleotide
gcgtcatacatggcctcagaaccgccacgaaccaccaccaga

SEQ ID NO: 501 - DNA, 42 bp, Artificial Sequence
  Description of Artificial Sequence Synthetic polynucleotide
gatattcacaaacattttgctcagtacccttgagtaacagtg

SEQ ID NO: 502 - DNA, 35 bp, Artificial Sequence
  Description of Artificial Sequence Synthetic polynucleotide
gccgccgccagcataacggggtcagtgcaggcgga

SEQ ID NO: 503 - DNA, 35 bp, Artificial Sequence
  Description of Artificial Sequence Synthetic polynucleotide
cgccaccttttgatgatacagagacgattggcctt

SEQ ID NO: 504 - DNA, 42 bp, Artificial Sequence
  Description of Artificial Sequence Synthetic polynucleotide
acataaaggtggcaaatatcagagagatagagcaagaaacaa

SEQ ID NO: 505 - DNA, 42 bp, Artificial Sequence
  Description of Artificial Sequence Synthetic polynucleotide
accgaggaaacgcatttacagagagaataaaatgaaaatagc

SEQ ID NO: 506 - DNA, 42 bp, Artificial Sequence
  Description of Artificial Sequence Synthetic polynucleotide
tgaaatagcaatagtttgtttaacgtcaaacataaaaacagg

SEQ ID NO: 507 - DNA, 42 bp, Artificial Sequence
  Description of Artificial Sequence Synthetic polynucleotide
ggtaattgagcgctacatataaaagaaagttaccagaaggaa

SEQ ID NO: 508 - DNA, 35 bp, Artificial Sequence
  Description of Artificial Sequence Synthetic polynucleotide
gaagcgcattagacgatagccgaacaaacgcaaag

SEQ ID NO: 509 - DNA, 35 bp, Artificial Sequence
  Description of Artificial Sequence Synthetic polynucleotide
acgatttctatcttaccgaaggaacaaagtcagag

SEQ ID NO: 510 - DNA, 42 bp, Artificial Sequence
  Description of Artificial Sequence Synthetic polynucleotide
ccggcttaggttggagaaaacaaaattattttaatggaaaca

SEQ ID NO: 511 - DNA, 42 bp, Artificial Sequence
  Description of Artificial Sequence Synthetic polynucleotide
ttgaaaacatagcgtgattgctttgaataacaataacggatt

SEQ ID NO: 512 - DNA, 42 bp, Artificial Sequence
  Description of Artificial Sequence Synthetic polynucleotide
gtacataaatcaattttacatcgggagaaccaagttacaaaa

SEQ ID NO: 513 - DNA, 42 bp, Artificial Sequence
  Description of Artificial Sequence Synthetic polynucleotide
tgaaacaaacatcagttatataactatattcccttagaatcc

SEQ ID NO: 514 - DNA, 35 bp, Artificial Sequence
  Description of Artificial Sequence Synthetic polynucleotide
tcgcgcagaggcgagctattaattaatttgtaaat

SEQ ID NO: 515 - DNA, 35 bp, Artificial Sequence
  Description of Artificial Sequence Synthetic polynucleotide
agtacctatatgtgagtgaatgcaaaagaagatga

SEQ ID NO: 516 - DNA, 46 bp, Artificial Sequence
  Description of Artificial Sequence Synthetic polynucleotide
ccaccacacccgccgcttttttttttttcggcctcaggaaaggggg

SEQ ID NO: 517 - DNA, 52 bp, Artificial Sequence
  Description of Artificial Sequence Synthetic polynucleotide
ggcgagaaaggatttttttttctccacaagctttttttttttgcgtattggg

SEQ ID NO: 518 - DNA, 46 bp, Artificial Sequence
  Description of Artificial Sequence Synthetic polynucleotide
cgggccgttttcacggttttttttttttcaacatacggggagaggc

SEQ ID NO: 519 - DNA, 52 bp, Artificial Sequence
  Description of Artificial Sequence Synthetic polynucleotide
gtgccccctgcattttttttttcaatcgtgctttttttttttaaacgatgct

SEQ ID NO: 520 - DNA, 46 bp, Artificial Sequence
  Description of Artificial Sequence Synthetic polynucleotide
cggccagtgccaagctttttttttttttgaagggatatgaagggta

SEQ ID NO: 521 - DNA, 46 bp, Artificial Sequence
  Description of Artificial Sequence Synthetic polynucleotide
tgataagaggtcatttttttttttttttatacatttccaataaagc

SEQ ID NO: 522 - DNA, 52 bp, Artificial Sequence
  Description of Artificial Sequence Synthetic polynucleotide
ttcgagcttcaattttttttttattcaggcatttttttttaacaacattatt

SEQ ID NO: 523 - DNA, 46 bp, Artificial Sequence
  Description of Artificial Sequence Synthetic polynucleotide
gattcaccagtcacactttttttttttttgacgagaaacgaactaa

SEQ ID NO: 524 - DNA, 52 bp, Artificial Sequence
  Description of Artificial Sequence Synthetic polynucleotide
ttgcaacaggaatttttttttttttaccagcttttttttttgttaaaattcg

SEQ ID NO: 525 - DNA, 52 bp, Artificial Sequence
  Description of Artificial Sequence Synthetic polynucleotide
taaacgttaatatttttttttagagatttagtttttttttagagcataaagc

SEQ ID NO: 526 - DNA, 46 bp, Artificial Sequence
  Description of Artificial Sequence Synthetic polynucleotide
acctaaatttaatggttttttttttttttcgccatatcctgtttat

SEQ ID NO: 527 - DNA, 52 bp, Artificial Sequence
  Description of Artificial Sequence Synthetic polynucleotide
gctgatgcaaatttttttttttcgtcattattttttttttatatacagtaac

SEQ ID NO: 528 - DNA, 46 bp, Artificial Sequence
  Description of Artificial Sequence Synthetic polynucleotide
tcaatagataatacatttttttttttttcctgattataacgtcaga

SEQ ID NO: 529 - DNA, 52 bp, Artificial Sequence
  Description of Artificial Sequence Synthetic polynucleotide
aatcaacagttgtttttttttcgaaccgaactttttttttttgtatcatcgc

SEQ ID NO: 530 - DNA, 46 bp, Artificial Sequence
  Description of Artificial Sequence Synthetic polynucleotide
caatcaataatcggctttttttttttttagacttttttacaacgga

SEQ ID NO: 531 - DNA, 46 bp, Artificial Sequence
  Description of Artificial Sequence Synthetic polynucleotide
accctcagaaccgccattttttttttttcgatctaaaaaatctcca

SEQ ID NO: 532 - DNA, 52 bp, Artificial Sequence
  Description of Artificial Sequence Synthetic polynucleotide
taagtgccgtcgtttttttttgtttttgacatttttttttctccctcagagc

SEQ ID NO: 533 - DNA, 46 bp, Artificial Sequence
  Description of Artificial Sequence Synthetic polynucleotide
acattcaaccgattgattttttttttttatagcagcacaccggaac

SEQ ID NO: 534 - DNA, 52 bp, Artificial Sequence
  Description of Artificial Sequence Synthetic polynucleotide
acaccacggaattttttttttaagcagggagttttttttttccaaataagaa

SEQ ID NO: 535 - DNA, 52 bp, Artificial Sequence
  Description of Artificial Sequence Synthetic polynucleotide
tattatttatcctttttttttcgcgaacaattttttttttaaaaggctccaa

SEQ ID NO: 536 - DNA, 52 bp, Artificial Sequence
  Description of Artificial Sequence Synthetic polynucleotide
gaagaaagcttgaggggatttttttgcctatttttttttctgccagcacgcg

SEQ ID NO: 537 - DNA, 52 bp, Artificial Sequence
  Description of Artificial Sequence Synthetic polynucleotide
gacgatccattatccgctttttacgtctgcagttttcggcttgacatatccc

SEQ ID NO: 538 - DNA, 38 bp, Artificial Sequence
  Description of Artificial Sequence Synthetic polynucleotide
tgctgcaaggagatagacttttgctacagggcgcgtac

SEQ ID NO: 539 - DNA, 52 bp, Artificial Sequence
  Description of Artificial Sequence Synthetic polynucleotide
gaaccagactagatttagttttcactatgcggttttaagggacattctggcc

SEQ ID NO: 540 - DNA, 32 bp, Artificial Sequence
  Description of Artificial Sequence Synthetic polynucleotide
cgctcatggttcagtgaatttttaaatagaat

SEQ ID NO: 541 - DNA, 30 bp, Artificial Sequence
  Description of Artificial Sequence Synthetic polynucleotide
ttttagagggtttttagagcttaattgctg

SEQ ID NO: 542 - DNA, 52 bp, Artificial Sequence
  Description of Artificial Sequence Synthetic polynucleotide
atcgcaagaacagtagggttttcctgaaacctttttaagtattagactttac

SEQ ID NO: 543 - DNA, 52 bp, Artificial Sequence
  Description of Artificial Sequence Synthetic polynucleotide
ggaattgaggagcggaatttttaaagaatcgcttttcattccaagaacgggt

SEQ ID NO: 544 - DNA, 38 bp, Artificial Sequence
  Description of Artificial Sequence Synthetic polynucleotide
caatagatatacagaggcttttccgtgtgataaataag

SEQ ID NO: 545 - DNA, 52 bp, Artificial Sequence
  Description of Artificial Sequence Synthetic polynucleotide
gggttgatacagccctcattttaggtcgagtgttttaaatattgacggaaat

SEQ ID NO: 546 - DNA, 32 bp, Artificial Sequence
  Description of Artificial Sequence Synthetic polynucleotide
tttattttgacgtcaccattttaccctccctt

SEQ ID NO: 547 - DNA, 30 bp, Artificial Sequence
  Description of Artificial Sequence Synthetic polynucleotide
cgcccatccgttttccaccctcagagccac

SEQ ID NO: 548 - DNA, 42 bp, Artificial Sequence
  Description of Artificial Sequence Synthetic polynucleotide
ctaaggaaaccgaggaattcgtaatcatagcctcctcacagt

SEQ ID NO: 549 - DNA, 42 bp, Artificial Sequence
  Description of Artificial Sequence Synthetic polynucleotide
caaagttaccaggtggtgcctaatgagtaacctgtcgtgcca

SEQ ID NO: 550 - DNA, 42 bp, Artificial Sequence
  Description of Artificial Sequence Synthetic polynucleotide
tgaggatccccgctctttccagtcgggagagctaactcacat

SEQ ID NO: 551 - DNA, 42 bp, Artificial Sequence
  Description of Artificial Sequence Synthetic polynucleotide
tcttaagacgctgacgaaacgtacagcgccgccacgggaacg

SEQ ID NO: 552 - DNA, 42 bp, Artificial Sequence
  Description of Artificial Sequence Synthetic polynucleotide
cgatagcttagaccagcggatcaaacttaaaaaatcccgtaa

SEQ ID NO: 553 - DNA, 42 bp, Artificial Sequence
  Description of Artificial Sequence Synthetic polynucleotide
gataacctcaccagtgtacatcgacataaaatttctgctcat

SEQ ID NO: 554 - DNA, 42 bp, Artificial Sequence
  Description of Artificial Sequence Synthetic polynucleotide
agtttgctaaacaatttcattccatataatgttttaaatatg

SEQ ID NO: 555 - DNA, 42 bp, Artificial Sequence
  Description of Artificial Sequence Synthetic polynucleotide
tctgtatgggatagattttcatttggggcaataaatcataca

SEQ ID NO: 556 - DNA, 42 bp, Artificial Sequence
  Description of Artificial Sequence Synthetic polynucleotide
caactaaagtacatgtagcattaacatccgcgagctgaaaag

SEQ ID NO: 557 - DNA, 42 bp, Artificial Sequence
  Description of Artificial Sequence Synthetic polynucleotide
taaatcaaaaatcagcaaaatcaccagtcaccgtcaccgact

SEQ ID NO: 558 - DNA, 42 bp, Artificial Sequence
  Description of Artificial Sequence Synthetic polynucleotide
agaatgaccatacctgcctttagcgtcattgccatcttttca

SEQ ID NO: 559 - DNA, 42 bp, Artificial Sequence
  Description of Artificial Sequence Synthetic polynucleotide
tgagccatttggaaccccttattagcgtgactgtagcgcgtt

SEQ ID NO: 560 - DNA, 42 bp, Artificial Sequence
  Description of Artificial Sequence Synthetic polynucleotide
ataaagagtctgtctatgcgttatacaacggaatcataatta

SEQ ID NO: 561 - DNA, 42 bp, Artificial Sequence
  Description of Artificial Sequence Synthetic polynucleotide
ggccaccgagtaatgaggcattttcgaggacgacgacaataa

SEQ ID NO: 562 - DNA, 42 bp, Artificial Sequence
  Description of Artificial Sequence Synthetic polynucleotide
ctagaaaaagccacagtaattctgtccaccagtaataagaga

SEQ ID NO: 563 - DNA, 42 bp, Artificial Sequence
  Description of Artificial Sequence Synthetic polynucleotide
ggtcagacgattggctggtgtgttcagcggtaaaggtttctt

SEQ ID NO: 564 - DNA, 42 bp, Artificial Sequence
  Description of Artificial Sequence Synthetic polynucleotide
aacggggtcagttctgcggctggtaatgaaatcgttaacggc

SEQ ID NO: 565 - DNA, 42 bp, Artificial Sequence
  Description of Artificial Sequence Synthetic polynucleotide
tcgccttgagtaacgtgccatcccacgcacggcagcaccgtc

SEQ ID NO: 566 - DNA, 42 bp, Artificial Sequence
  Description of Artificial Sequence Synthetic polynucleotide
gaacaccggaatcaccccggttgataatgaacggtaatcgta

SEQ ID NO: 567 - DNA, 42 bp, Artificial Sequence
  Description of Artificial Sequence Synthetic polynucleotide
cagaggcattttacacaagagaatcgatcagaaaagccccaa

SEQ ID NO: 568 - DNA, 42 bp, Artificial Sequence
  Description of Artificial Sequence Synthetic polynucleotide
atcgagccagtaatgtcaaatcaccatctgagaaaggccgga

SEQ ID NO: 569 - DNA, 42 bp, Artificial Sequence
  Description of Artificial Sequence Synthetic polynucleotide
aaggcttgcagggaagaggctgagactctatttcggaaccta

SEQ ID NO: 570 - DNA, 42 bp, Artificial Sequence
  Description of Artificial Sequence Synthetic polynucleotide
aaaatacgtaatcttaatgccccctgccctcaagagaaggat

SEQ ID NO: 571 - DNA, 42 bp, Artificial Sequence
  Description of Artificial Sequence Synthetic polynucleotide
ccgccactacgaaggcagtctctgaattaaagccagaatgga

SEQ ID NO: 572 - DNA, 42 bp, Artificial Sequence
  Description of Artificial Sequence Synthetic polynucleotide
gagccccagcaggcagtatgttagcaaacccaaaagaactgg

SEQ ID NO: 573 - DNA, 42 bp, Artificial Sequence
  Description of Artificial Sequence Synthetic polynucleotide
acgtcaaagggcccataataacggaatacgtagaaaatacat

SEQ ID NO: 574 - DNA, 42 bp, Artificial Sequence
  Description of Artificial Sequence Synthetic polynucleotide
aggaaaaaccgtcttaagcccaataataaacccacaagaatt

SEQ ID NO: 575 - DNA, 42 bp, Artificial Sequence
  Description of Artificial Sequence Synthetic polynucleotide
ttcaagtaccgcactaggtctgagagacgacgctgagaagag

SEQ ID NO: 576 - DNA, 42 bp, Artificial Sequence
  Description of Artificial Sequence Synthetic polynucleotide
gacttgcgggagccatagcttagattaatacctttttaacct

SEQ ID NO: 577 - DNA, 42 bp, Artificial Sequence
  Description of Artificial Sequence Synthetic polynucleotide
cggttttgaagcctatttgaattaccttattacatttaacaa

SEQ ID NO: 578 - DNA, 7704 bp, M13 bacteriophage
aatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcccgcctaaagagatagggccgaccaacgtcagacgcactcaatgtcacctggctgggtttgtcaggtgtcgtgactctgggcaagaccggcaactgctgttgttgagttccccatggtcggtctcagtgggaactgcttccaacttccgtctttatcgaggtaacaagcaccacgtagcttaagccctgtttactcattacaccaaccaggaggtcagagttcggagaaatgatttatgtgaaatgcgtcagccgattcaaggcccctatattcgtgcccaccgacgagttgcttacagatggcagggccgcactgtcggtatcatagagtcactccagggcgagcgtaaatagattagaagcggggttattttggcgggacattgtcataaggttgacaattcagcactaaggacacttaagtcgtgcgcatgaattcacaaccacttagaagaacatccaccctggcttctcctgagaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgtt

SEQ ID NO: 579 - DNA, 43 bp, Artificial Sequence
  Description of Artificial Sequence Synthetic polynucleotide
aggtgttgggcgaagcccaacgaatatagggtgcttgttacct

SEQ ID NO: 580 - DNA, 43 bp, Artificial Sequence
  Description of Artificial Sequence Synthetic polynucleotide
cggctatgggcgaagcccaaaaccaggcaaagcaaggcgatta

SEQ ID NO: 581 - DNA, 43 bp, Artificial Sequence
  Description of Artificial Sequence Synthetic polynucleotide
tactgttgggcgaagcccatgtaccccggttgttaaatcagct

SEQ ID NO: 582 - DNA, 43 bp, Artificial Sequence
  Description of Artificial Sequence Synthetic polynucleotide
gtcccttgggcgaagcccacgaaagacttcaataagaggtcat

SEQ ID NO: 583 - DNA, 43 bp, Artificial Sequence
  Description of Artificial Sequence Synthetic polynucleotide
atgtgttgggcgaagcccaggaacaacattagagcaacactat

SEQ ID NO: 584 - DNA, 43 bp, Artificial Sequence
  Description of Artificial Sequence Synthetic polynucleotide
aaagtatgggcgaagcccagagggagggaagaccattaccatt

SEQ ID NO: 585 - DNA, 43 bp, Artificial Sequence
  Description of Artificial Sequence Synthetic polynucleotide
acaatgtgggcgaagcccaaacaaagtcagattaccgaagccc

SEQ ID NO: 586 - DNA, 43 bp, Artificial Sequence
  Description of Artificial Sequence Synthetic polynucleotide
attagttgggcgaagcccaaagcaagccgttgcgaacctcccg

SEQ ID NO: 587 - DNA, 43 bp, Artificial Sequence
  Description of Artificial Sequence Synthetic polynucleotide
ccatcttgggcgaagcccatttgaattaccttgaaaacatagc

SEQ ID NO: 588 - DNA, 43 bp, Artificial Sequence
  Description of Artificial Sequence Synthetic polynucleotide
gtgcattgggcgaagcccatcattttgcggaacttctgaataa

SEQ ID NO: 589 - DNA, 43 bp, Artificial Sequence
  Description of Artificial Sequence Synthetic polynucleotide
ctaatgtgggcgaagcccatgatagccctaatgagagccagca

SEQ ID NO: 590 - DNA, 43 bp, Artificial Sequence
  Description of Artificial Sequence Synthetic polynucleotide
ggaacatgggcgaagcccagtaaagcctgggcggccaacgcgc

SEQ ID NO: 591 - DNA, 43 bp, Artificial Sequence
  Description of Artificial Sequence Synthetic polynucleotide
attgactgggcgaagcccacaggcaaggcaagcctttatttca

SEQ ID NO: 592 - DNA, 43 bp, Artificial Sequence
  Description of Artificial Sequence Synthetic polynucleotide
tgttcctgggcgaagcccacgtcaaagggcgcctaaagggagc

SEQ ID NO: 593 - DNA, 43 bp, Artificial Sequence
  Description of Artificial Sequence Synthetic polynucleotide
tggaagtgggcgaagcccaatcagcttgcttggcttgcaggga

SEQ ID NO: 594 - DNA, 43 bp, Artificial Sequence
  Description of Artificial Sequence Synthetic polynucleotide
acgtcgtgggcgaagcccatcctgagaagtgttgcctgagtag

SEQ ID NO: 595 - DNA, 43 bp, Artificial Sequence
  Description of Artificial Sequence Synthetic polynucleotide
taactctgggcgaagcccaccgtactcaggacccatgtaccgt

SEQ ID NO: 596 - DNA, 43 bp, Artificial Sequence
  Description of Artificial Sequence Synthetic polynucleotide
catgcttgggcgaagcccaaattactagaaaaacgccaacatg

SEQ ID NO: 597 - DNA, 43 bp, Artificial Sequence
  Description of Artificial Sequence Synthetic polynucleotide
acatactgggcgaagcccaagagccgccgccctgaatttaccg

SEQ ID NO: 598 - DNA, 43 bp, Artificial Sequence
  Description of Artificial Sequence Synthetic polynucleotide
gaaacctgggcgaagcccaaattgtgtcgaaaacggtgtacag

SEQ ID NO: 599 - DNA, 35 bp, Artificial Sequence
  Description of Artificial Sequence Synthetic polynucleotide
taacgccagggtgaagccagggtgattttagcgag

SEQ ID NO: 600 - DNA, 35 bp, Artificial Sequence
  Description of Artificial Sequence Synthetic polynucleotide
taaccaataggacattaaatgtgaattttaagaaa

SEQ ID NO: 601 - DNA, 35 bp, Artificial Sequence
  Description of Artificial Sequence Synthetic polynucleotide
gataaaaattttttcaaaagggtgattttagcctg

SEQ ID NO: 602 - DNA, 35 bp, Artificial Sequence
  Description of Artificial Sequence Synthetic polynucleotide
ctcgtttaccaggagggggtaataattttagctgc

SEQ ID NO: 603 - DNA, 35 bp, Artificial Sequence
  Description of Artificial Sequence Synthetic polynucleotide
gcataggctggccaacgtaacaaaattttagagga

SEQ ID NO: 604 - DNA, 35 bp, Artificial Sequence
  Description of Artificial Sequence Synthetic polynucleotide
gaaaagtaagcaaatacccaaaagattttagctaa

SEQ ID NO: 605 - DNA, 35 bp, Artificial Sequence
  Description of Artificial Sequence Synthetic polynucleotide
ggaggttttgaaaatcttaccaacattttagacaa

SEQ ID NO: 606 - DNA, 35 bp, Artificial Sequence
  Description of Artificial Sequence Synthetic polynucleotide
ggcagaggcatttgtccagacgacattttaagtgc

SEQ ID NO: 607 - DNA, 35 bp, Artificial Sequence
  Description of Artificial Sequence Synthetic polynucleotide
gttagaacctactcaggtttaacgattttaagttg

SEQ ID NO: 608 - DNA, 35 bp, Artificial Sequence
  Description of Artificial Sequence Synthetic polynucleotide
aaaaatctaaagtggcaaatcaacattttatacct

SEQ ID NO: 609 - DNA, 35 bp, Artificial Sequence
  Description of Artificial Sequence Synthetic polynucleotide
aaactatcggcccgctcatggaaaattttattttg

SEQ ID NO: 610 - DNA, 35 bp, Artificial Sequence
  Description of Artificial Sequence Synthetic polynucleotide
gacggaagttgggtcttgcccagaattttagatgt

SEQ ID NO: 611 - DNA, 35 bp, Artificial Sequence
  Description of Artificial Sequence Synthetic polynucleotide
ggcggtttgcgtttgcccttcaccattttagtcac

SEQ ID NO: 612 - DNA, 35 bp, Artificial Sequence
  Description of Artificial Sequence Synthetic polynucleotide
gatggcttagagaagtacggtgtcattttagtaaa

SEQ ID NO: 613 - DNA, 35 bp, Artificial Sequence
  Description of Artificial Sequence Synthetic polynucleotide
tagagcttgacgggcgctagggcgattttatggaa

SEQ ID NO: 614 - DNA, 35 bp, Artificial Sequence
  Description of Artificial Sequence Synthetic polynucleotide
gtttcgtcaccaaacgatctaaagattttaggctt

SEQ ID NO: 615 - DNA, 35 bp, Artificial Sequence
  Description of Artificial Sequence Synthetic polynucleotide
tagattaagacgtttttaacctccattttatcaga

SEQ ID NO: 616 - DNA, 35 bp, Artificial Sequence
  Description of Artificial Sequence Synthetic polynucleotide
aagcgtcatacagccttgagtaacattttacagac

SEQ ID NO: 617 - DNA, 35 bp, Artificial Sequence
  Description of Artificial Sequence Synthetic polynucleotide
ccggaaacgtcatgcctttagcgtattttaaactg

SEQ ID NO: 618 - DNA, 35 bp, Artificial Sequence
  Description of Artificial Sequence Synthetic polynucleotide
ccgcttttgcggtacagaggctttattttactggc

SEQ ID NO: 619 - DNA, 28 bp, Artificial Sequence
  Description of Artificial Sequence Synthetic polynucleotide
tcttctaagtggtttacgtgggccttga

SEQ ID NO: 620 - DNA, 28 bp, Artificial Sequence
  Description of Artificial Sequence Synthetic polynucleotide
taacaacccgtcggatgtgctgcgccat

SEQ ID NO: 621 - DNA, 28 bp, Artificial Sequence
  Description of Artificial Sequence Synthetic polynucleotide
ggccggagacagtcaatttttgataatc

SEQ ID NO: 622 - DNA, 28 bp, Artificial Sequence
  Description of Artificial Sequence Synthetic polynucleotide
atgtttagactggaccttttgaatatcg

SEQ ID NO: 623 - DNA, 28 bp, Artificial Sequence
  Description of Artificial Sequence Synthetic polynucleotide
tcattcagtgaataatagtaattacagg

SEQ ID NO: 624 - DNA, 28 bp, Artificial Sequence
  Description of Artificial Sequence Synthetic polynucleotide
gcatgattaagactcagtagcgtaaata

SEQ ID NO: 625 - DNA, 28 bp, Artificial Sequence
  Description of Artificial Sequence Synthetic polynucleotide
cgagcgtctttccaagctatcgggtaat

SEQ ID NO: 626 - DNA, 28 bp, Artificial Sequence
  Description of Artificial Sequence Synthetic polynucleotide
taaacaacatgttccgttttatttattt

SEQ ID NO: 627 - DNA, 28 bp, Artificial Sequence
  Description of Artificial Sequence Synthetic polynucleotide
tgaatatacagtaagaatccttttttaa

SEQ ID NO: 628 - DNA, 28 bp, Artificial Sequence
  Description of Artificial Sequence Synthetic polynucleotide
aaaggaattgaggaggattatacaaaga

SEQ ID NO: 629 - DNA, 28 bp, Artificial Sequence
  Description of Artificial Sequence Synthetic polynucleotide
acattttgacgctcgccacgcaacatcg

SEQ ID NO: 630 - DNA, 28 bp, Artificial Sequence
  Description of Artificial Sequence Synthetic polynucleotide
gacacctgacaaacaatgaatgtgccta

SEQ ID NO: 631 - DNA, 28 bp, Artificial Sequence
  Description of Artificial Sequence Synthetic polynucleotide
gccctgagagagttgggagaaagaatta

SEQ ID NO: 632 - DNA, 28 bp, Artificial Sequence
  Description of Artificial Sequence Synthetic polynucleotide
gtttcattccatattcggaacaaaaacc

SEQ ID NO: 633 - DNA, 28 bp, Artificial Sequence
  Description of Artificial Sequence Synthetic polynucleotide
aagtgtagcggtcatcgctgatcgaggt

SEQ ID NO: 634 - DNA, 28 bp, Artificial Sequence
  Description of Artificial Sequence Synthetic polynucleotide
tcgtctttccagacacatcactttttat

SEQ ID NO: 635 - DNA, 28 bp, Artificial Sequence
  Description of Artificial Sequence Synthetic polynucleotide
aggttgggttatatataggaaggtttag

SEQ ID NO: 636 - DNA, 28 bp, Artificial Sequence
  Description of Artificial Sequence Synthetic polynucleotide
ccgtataaacagttatttaacaagcctg

SEQ ID NO: 637 - DNA, 28 bp, Artificial Sequence
  Description of Artificial Sequence Synthetic polynucleotide
tgtagcgcgttttcgcagtctagcattg

SEQ ID NO: 638 - DNA, 28 bp, Artificial Sequence
  Description of Artificial Sequence Synthetic polynucleotide
ctaaagactttttcacagatgatccgcg

SEQ ID NO: 639 - DNA, 40 bp, Artificial Sequence
  Description of Artificial Sequence Synthetic polynucleotide
ctcaggatttcccaaagcaactcgtcggtgggcacaccta

SEQ ID NO: 640 - DNA, 40 bp, Artificial Sequence
  Description of Artificial Sequence Synthetic polynucleotide
tcatcaaacgccatgcaccgcttctggtgccggtagccga

SEQ ID NO: 641 - DNA, 40 bp, Artificial Sequence
  Description of Artificial Sequence Synthetic polynucleotide
gtaaagatagaaccaactagcatgtcaatcataacagtaa

SEQ ID NO: 642 - DNA, 40 bp, Artificial Sequence
  Description of Artificial Sequence Synthetic polynucleotide
tttgccaacgacgaaaaagattaagaggaagccagggaca

SEQ ID NO: 643 - DNA, 40 bp, Artificial Sequence
  Description of Artificial Sequence Synthetic polynucleotide
cccaaattgaccttgttaataaaacgaactaacacacata

SEQ ID NO: 644 - DNA, 40 bp, Artificial Sequence
  Description of Artificial Sequence Synthetic polynucleotide
ataacgggatagccgggcgacattcaaccgatttacttta

SEQ ID NO: 645 - DNA, 40 bp, Artificial Sequence
  Description of Artificial Sequence Synthetic polynucleotide
tatcctggccttaagaattaactgaacaccctgcattgta

SEQ ID NO: 646 - DNA, 40 bp, Artificial Sequence
  Description of Artificial Sequence Synthetic polynucleotide
gtaattcttcgagctaccgcactcatcgagaacactaata

SEQ ID NO: 647 - DNA, 40 bp, Artificial Sequence
  Description of Artificial Sequence Synthetic polynucleotide
tagatttcatatcaattacatttaacaatttcaagatgga

SEQ ID NO: 648 - DNA, 40 bp, Artificial Sequence
  Description of Artificial Sequence Synthetic polynucleotide
ggtcagtcatcaccaaaagtttgagtaacattaatgcaca

SEQ ID NO: 649 - DNA, 40 bp, Artificial Sequence
  Description of Artificial Sequence Synthetic polynucleotide
ggaaaaattgctggtagtctttaatgcgcgaaccattaga

SEQ ID NO: 650 - DNA, 40 bp, Artificial Sequence
  Description of Artificial Sequence Synthetic polynucleotide
gttgccgaagcagtcgagccggaagcataaagttgttcca

SEQ ID NO: 651 - DNA, 40 bp, Artificial Sequence
  Description of Artificial Sequence Synthetic polynucleotide
cagctgaattgggctaacatccaataaatcatagtcaata

SEQ ID NO: 652 - DNA, 40 bp, Artificial Sequence
  Description of Artificial Sequence Synthetic polynucleotide
gcaactacttaatttaaagaacgtggactccaaggaacaa

SEQ ID NO: 653 - DNA, 40 bp, Artificial Sequence
  Description of Artificial Sequence Synthetic polynucleotide
aggagcggggaaagcctttaattgtatcggtttcttccaa

SEQ ID NO: 654 - DNA, 40 bp, Artificial Sequence
  Description of Artificial Sequence Synthetic polynucleotide
ttagcgtgtacaaacaggaacggtacgccagaacgacgta

SEQ ID NO: 655 - DNA, 40 bp, Artificial Sequence
  Description of Artificial Sequence Synthetic polynucleotide
gactaccctgagaagcccggaataggtgtatcagagttaa

SEQ ID NO: 656 - DNA, 40 bp, Artificial Sequence
  Description of Artificial Sequence Synthetic polynucleotide
ggtcagttggctttgaataaacaccggaatcatagcatga

SEQ ID NO: 657 - DNA, 40 bp, Artificial Sequence
  Description of Artificial Sequence Synthetic polynucleotide
tcaagttccaatgacgccaccagaaccaccaccgtatgta

SEQ ID NO: 658 - DNA, 40 bp, Artificial Sequence
  Description of Artificial Sequence Synthetic polynucleotide
caacggcgatcgtctttgtatcatcgcctgataggtttca

SEQ ID NO: 659 - DNA, 42 bp, Artificial Sequence
  Description of Artificial Sequence Synthetic polynucleotide
atcggctcatctgtgtcacgacgttgtaaaacgaacagtgcg

SEQ ID NO: 660 - DNA, 42 bp, Artificial Sequence
  Description of Artificial Sequence Synthetic polynucleotide
tcgccatctttccgcaaaaataattcgcgtctggtcgcactc

SEQ ID NO: 661 - DNA, 42 bp, Artificial Sequence
  Description of Artificial Sequence Synthetic polynucleotide
agaaaagatcgtaactcatatattttaaatgcagaatcgatg

SEQ ID NO: 662 - DNA, 42 bp, Artificial Sequence
  Description of Artificial Sequence Synthetic polynucleotide
cgttttatgcatcataaaaaccaaaatagcgagaagaagcaa

SEQ ID NO: 663 - DNA, 42 bp, Artificial Sequence
  Description of Artificial Sequence Synthetic polynucleotide
tagaaagaatctaccatcaagagtaatcttgacaaacgttgg

SEQ ID NO: 664 - DNA, 42 bp, Artificial Sequence
  Description of Artificial Sequence Synthetic polynucleotide
ttgacgggacaaaagaacaaagttaccagaaggcattaccag

SEQ ID NO: 665 - DNA, 42 bp, Artificial Sequence
  Description of Artificial Sequence Synthetic polynucleotide
tgagcgcgacgggaatcaagattagttgctattttagggaag

SEQ ID NO: 666 - DNA, 42 bp, Artificial Sequence
  Description of Artificial Sequence Synthetic polynucleotide
tcatcgtaaccaagcagtaataagagaatataataaagaacg

SEQ ID NO: 667 - DNA, 42 bp, Artificial Sequence
  Description of Artificial Sequence Synthetic polynucleotide
tggaaacaaaattaaaattatttgcacgtaaaacgaacatca

SEQ ID NO: 668 - DNA, 42 bp, Artificial Sequence
  Description of Artificial Sequence Synthetic polynucleotide
aaccacctaattttttgctgaacctcaaatatcgatgcccga

SEQ ID NO: 669 - DNA, 42 bp, Artificial Sequence
  Description of Artificial Sequence Synthetic polynucleotide
ccattaatggctattaatatccagaacaatattctacaatat

SEQ ID NO: 670 - DNA, 42 bp, Artificial Sequence
  Description of Artificial Sequence Synthetic polynucleotide
atgagtgcaacatatcccactgagaccgaccatcgctcacaa

SEQ ID NO: 671 - DNA, 42 bp, Artificial Sequence
  Description of Artificial Sequence Synthetic polynucleotide
gcaaaatgtagcatgccagggtggtttttctttaattctact

SEQ ID NO: 672 - DNA, 42 bp, Artificial Sequence
  Description of Artificial Sequence Synthetic polynucleotide
gtctatcccactatgctgaatataatgctgtagcatttggaa

SEQ ID NO: 673 - DNA, 42 bp, Artificial Sequence
  Description of Artificial Sequence Synthetic polynucleotide
gaatttcaaaggagccggcgaacgtggcgagaagaaaaaaaa

SEQ ID NO: 674 - DNA, 42 bp, Artificial Sequence
  Description of Artificial Sequence Synthetic polynucleotide
aatcagtttttagactacaacgcctgtagcattacgccgatt

SEQ ID NO: 675 - DNA, 42 bp, Artificial Sequence
  Description of Artificial Sequence Synthetic polynucleotide
taccgccaagtatagagtcaatagtgaatttataggagaggg

SEQ ID NO: 676 - DNA, 42 bp, Artificial Sequence
  Description of Artificial Sequence Synthetic polynucleotide
tttagtataaataatgatgatacaggagtgtacttgataaat

SEQ ID NO: 677 - DNA, 42 bp, Artificial Sequence
  Description of Artificial Sequence Synthetic polynucleotide
acaggagtcagagcaaccatcgatagcagcaccagcagagcc

SEQ ID NO: 678 - DNA, 42 bp, Artificial Sequence
  Description of Artificial Sequence Synthetic polynucleotide
acctgctacggagaaccctcagcagcgaaagacagcgaaaca

SEQ ID NO: 679 - DNA, 42 bp, Artificial Sequence
  Description of Artificial Sequence Synthetic polynucleotide
gccctgcgacgcataacagggcttaagcgtgaattaagcttt

SEQ ID NO: 680 - DNA, 42 bp, Artificial Sequence
  Description of Artificial Sequence Synthetic polynucleotide
cagccagtcaggctctggcgaaagggggattctccccagctt

SEQ ID NO: 681 - DNA, 42 bp, Artificial Sequence
  Description of Artificial Sequence Synthetic polynucleotide
aacggtaccccaaataaaattcgcattaaaatcactgtgtag

SEQ ID NO: 682 - DNA, 42 bp, Artificial Sequence
  Description of Artificial Sequence Synthetic polynucleotide
agcggatattcgagtacctttaattgcttagcgtcaagaagt

SEQ ID NO: 683 - DNA, 42 bp, Artificial Sequence
  Description of Artificial Sequence Synthetic polynucleotide
gaagaaaattcatcaggaattacgaggcaggcttgttcatta

SEQ ID NO: 684 - DNA, 42 bp, Artificial Sequence
  Description of Artificial Sequence Synthetic polynucleotide
cgccaaaaaattatgccagcaaaatcacccttattcgcaata

SEQ ID NO: 685 - DNA, 42 bp, Artificial Sequence
  Description of Artificial Sequence Synthetic polynucleotide
cgcattataatatcaatgaaatagcaatgagcctaacaattt

SEQ ID NO: 686 - DNA, 42 bp, Artificial Sequence
  Description of Artificial Sequence Synthetic polynucleotide
ggtattaaggaatcctaagaacgcgaggagctaataggtaaa

SEQ ID NO: 687 - DNA, 42 bp, Artificial Sequence
  Description of Artificial Sequence Synthetic polynucleotide
agaaaacagtacatttaattttcccttacagtaccaattgcg

SEQ ID NO: 688 - DNA, 42 bp, Artificial Sequence
  Description of Artificial Sequence Synthetic polynucleotide
acgttatagaaggaaatcctgattgtttaggttataatatct

SEQ ID NO: 689 - DNA, 42 bp, Artificial Sequence
  Description of Artificial Sequence Synthetic polynucleotide
ttttgaaaaataccccgcctgcaacagtaatcgtctgcaaca

SEQ ID NO: 690 - DNA, 42 bp, Artificial Sequence
  Description of Artificial Sequence Synthetic polynucleotide
ttccacaagctaacgtgccagctgcattccagccaaacagca

SEQ ID NO: 691 - DNA, 42 bp, Artificial Sequence
  Description of Artificial Sequence Synthetic polynucleotide
aatagtataagcaatgtaatacttttgcgcagcaacgggcaa

SEQ ID NO: 692 - DNA, 42 bp, Artificial Sequence
  Description of Artificial Sequence Synthetic polynucleotide
caagagtagggcgacgtaaagcactaaaaacagtttaaatat

SEQ ID NO: 693 - DNA, 42 bp, Artificial Sequence
  Description of Artificial Sequence Synthetic polynucleotide
ggctccattaaacaaccgatatattcggcgctgcgaagcgaa

SEQ ID NO: 694 - DNA, 42 bp, Artificial Sequence
  Description of Artificial Sequence Synthetic polynucleotide
aaagggagaggccatttgattagtaatagttagtactcatag

SEQ ID NO: 695 - DNA, 42 bp, Artificial Sequence
  Description of Artificial Sequence Synthetic polynucleotide
ttgatataccctcaggatagcaagcccaaactatatctgaga

SEQ ID NO: 696 - DNA, 42 bp, Artificial Sequence
  Description of Artificial Sequence Synthetic polynucleotide
aaggcgttcatatgttgagaatcgccataatgcccttaacgg

SEQ ID NO: 697 - DNA, 42 bp, Artificial Sequence
  Description of Artificial Sequence Synthetic polynucleotide
accacccgttgaggccagaatggaaagcatcggcagacagaa

SEQ ID NO: 698 - DNA, 42 bp, Artificial Sequence
  Description of Artificial Sequence Synthetic polynucleotide
aagtacaccatgttaactttgaaagaggatgaggaagggtag

SEQ ID NO: 699 - DNA, 42 bp, Artificial Sequence
  Description of Artificial Sequence Synthetic polynucleotide
aaacggccagtgcccatgcgcacgacttatgagtattcacat

SEQ ID NO: 700 - DNA, 42 bp, Artificial Sequence
  Description of Artificial Sequence Synthetic polynucleotide
tcgccttcctgtaggtgggaacaaacggacgccaggcgcaac

SEQ ID NO: 701 - DNA, 42 bp, Artificial Sequence
  Description of Artificial Sequence Synthetic polynucleotide
ttatgcctgagtaacatcaatatgatatattttgtaacagga

SEQ ID NO: 702 - DNA, 42 bp, Artificial Sequence
  Description of Artificial Sequence Synthetic polynucleotide
agaggcttttgcaacaatactgcggaattagagagcttcaaa

SEQ ID NO: 703 - DNA, 42 bp, Artificial Sequence
  Description of Artificial Sequence Synthetic polynucleotide
ttaagaaccggataccctgacgagaaaccgccaaaagttgag

SEQ ID NO: 704 - DNA, 42 bp, Artificial Sequence
  Description of Artificial Sequence Synthetic polynucleotide
cgaaaccgaggaaaacgcagtatgttagaattagatcattaa

SEQ ID NO: 705 - DNA, 42 bp, Artificial Sequence
  Description of Artificial Sequence Synthetic polynucleotide
ttttgcacccagctatttgccagttacaaagaaacagagaga

SEQ ID NO: 706 - DNA, 42 bp, Artificial Sequence
  Description of Artificial Sequence Synthetic polynucleotide
ccagtaccgacaaagcagaacgcgcctgcggtattattaccg

SEQ ID NO: 707 - DNA, 42 bp, Artificial Sequence
  Description of Artificial Sequence Synthetic polynucleotide
atcagaaataaagattttacatcgggagctattaaaaatcaa

SEQ ID NO: 708 - DNA, 42 bp, Artificial Sequence
  Description of Artificial Sequence Synthetic polynucleotide
ttaaaccctcaatcctaaaatatctttatcaatatgcggaat

SEQ ID NO: 709 - DNA, 42 bp, Artificial Sequence
  Description of Artificial Sequence Synthetic polynucleotide
gattattattaacagaacgaaccaccagggcacagggccaac

SEQ ID NO: 710 - DNA, 42 bp, Artificial Sequence
  Description of Artificial Sequence Synthetic polynucleotide
ttgagtgacctgtctcacattaattgcgttatccgagctcga

SEQ ID NO: 711 - DNA, 42 bp, Artificial Sequence
  Description of Artificial Sequence Synthetic polynucleotide
acgctggatgaccctaaagcctcagagcgcatcaataacctg

SEQ ID NO: 712 - DNA, 42 bp, Artificial Sequence
  Description of Artificial Sequence Synthetic polynucleotide
aattctggaggtgctggcccactacgtggttccagaaatccc

SEQ ID NO: 713 - DNA, 42 bp, Artificial Sequence
  Description of Artificial Sequence Synthetic polynucleotide
accacacacgcatagcttgataccgataatctccaaaggaac

SEQ ID NO: 714 - DNA, 42 bp, Artificial Sequence
  Description of Artificial Sequence Synthetic polynucleotide
tttctgtatacttcccgagtaaaagagtacaggaggagcacg

SEQ ID NO: 715 - DNA, 42 bp, Artificial Sequence
  Description of Artificial Sequence Synthetic polynucleotide
gctgatgttttcaggaaccgccaccctctgccgtcagaagga

SEQ ID NO: 716 - DNA, 42 bp, Artificial Sequence
  Description of Artificial Sequence Synthetic polynucleotide
atttcggggcttaacgttatacaaattcaccgtgtttagtta

SEQ ID NO: 717 - DNA, 42 bp, Artificial Sequence
  Description of Artificial Sequence Synthetic polynucleotide
tcatagcattaaagcaggtcagacgattccaccctagccacc

SEQ ID NO: 718 - DNA, 42 bp, Artificial Sequence
  Description of Artificial Sequence Synthetic polynucleotide
attaaacactgaccacttagccggaacgccaagcggcaaaag

SEQ ID NO: 719 - DNA, 42 bp, Artificial Sequence
  Description of Artificial Sequence Synthetic polynucleotide
ggtcagttacattgagggacattaccgccagccattgaaatg

SEQ ID NO: 720 - DNA, 42 bp, Artificial Sequence
  Description of Artificial Sequence Synthetic polynucleotide
tcgggaacgtctgacccgggtacggggaactcaacggtgaca

SEQ ID NO: 721 - DNA, 42 bp, Artificial Sequence
  Description of Artificial Sequence Synthetic polynucleotide
aaacatttttgccccaaatggtctcaccagtgagagcggtcc

SEQ ID NO: 722 - DNA, 42 bp, Artificial Sequence
  Description of Artificial Sequence Synthetic polynucleotide
tggggtccgaacgacgaaatcggctcaacatgtttgattccc

SEQ ID NO: 723 - DNA, 42 bp, Artificial Sequence
  Description of Artificial Sequence Synthetic polynucleotide
atcgcccccgccgcgtgagaataaggaagggaagacgtaacc

SEQ ID NO: 724 - DNA, 42 bp, Artificial Sequence
  Description of Artificial Sequence Synthetic polynucleotide
tgtagcaatgggatgttgctttgccacagacagccaatgaat

SEQ ID NO: 725 - DNA, 42 bp, Artificial Sequence
  Description of Artificial Sequence Synthetic polynucleotide
accctcacaaatccgactcctcacaaaatcataggtgtaaat

SEQ ID NO: 726 - DNA, 42 bp, Artificial Sequence
  Description of Artificial Sequence Synthetic polynucleotide
acagtagaacctattcaaatatatggtaataagttcctgcct

SEQ ID NO: 727 - DNA, 42 bp, Artificial Sequence
  Description of Artificial Sequence Synthetic polynucleotide
aatcctcccccttaaccggaaccgtaatcagtagcttttcgg

SEQ ID NO: 728 - DNA, 42 bp, Artificial Sequence
  Description of Artificial Sequence Synthetic polynucleotide
gaaccgagggtaaaaaacgaaagagcatcggaacgagtttcc

SEQ ID NO: 729 - DNA, 29 bp, Artificial Sequence
  Description of Artificial Sequence Synthetic polynucleotide
agagatagaacccttctgatcacgaccag

SEQ ID NO: 730 - DNA, 29 bp, Artificial Sequence
  Description of Artificial Sequence Synthetic polynucleotide
attcgtaatcatggtcatcactctttagg

SEQ ID NO: 731 - DNA, 29 bp, Artificial Sequence
  Description of Artificial Sequence Synthetic polynucleotide
tttagctatattttcattaccattagata

SEQ ID NO: 732 - DNA, 29 bp, Artificial Sequence
  Description of Artificial Sequence Synthetic polynucleotide
ttataaatcaaaagaatacctgtttgatg

SEQ ID NO: 733 - DNA, 29 bp, Artificial Sequence
  Description of Artificial Sequence Synthetic polynucleotide
aactaaaggaattgcgaattcaacagttt

SEQ ID NO: 734 - DNA, 29 bp, Artificial Sequence
  Description of Artificial Sequence Synthetic polynucleotide
tataacgtgctttcctcgtacagggcgcg

SEQ ID NO: 735 - DNA, 29 bp, Artificial Sequence
  Description of Artificial Sequence Synthetic polynucleotide
ttaggattagcggggtttaaagtattaag

SEQ ID NO: 736 - DNA, 29 bp, Artificial Sequence
  Description of Artificial Sequence Synthetic polynucleotide
atttcatcttctgacctagaacgcgagaa

SEQ ID NO: 737 - DNA, 29 bp, Artificial Sequence
  Description of Artificial Sequence Synthetic polynucleotide
accggaaccgcctccctctatttcataat

SEQ ID NO: 738 - DNA, 29 bp, Artificial Sequence
  Description of Artificial Sequence Synthetic polynucleotide
aatacactaaaacactcactcgaaggcac

SEQ ID NO: 739 - DNA, 35 bp, Artificial Sequence
  Description of Artificial Sequence Synthetic polynucleotide
taataaagcagattcaccagtagcagaggtgaggc

SEQ ID NO: 740 - DNA, 35 bp, Artificial Sequence
  Description of Artificial Sequence Synthetic polynucleotide
cgggatccgttggtcggccctaccccgctttccag

SEQ ID NO: 741 - DNA, 35 bp, Artificial Sequence
  Description of Artificial Sequence Synthetic polynucleotide
catttcgcagcaggcgaaaatgccggttgtaccaa

SEQ ID NO: 742 - DNA, 35 bp, Artificial Sequence
  Description of Artificial Sequence Synthetic polynucleotide
gtggttcgtagatttagtttgtgaatcaagttttt

SEQ ID NO: 743 - DNA, 35 bp, Artificial Sequence
  Description of Artificial Sequence Synthetic polynucleotide
cagcggagcttaatgcgccgcttatgacaacaacc

SEQ ID NO: 744 - DNA, 35 bp, Artificial Sequence
  Description of Artificial Sequence Synthetic polynucleotide
tactatgtttgctaaacaacttacaaattaaccgt

SEQ ID NO: 745 - DNA, 35 bp, Artificial Sequence
  Description of Artificial Sequence Synthetic polynucleotide
aggctgaaatcgcaagacaaaaactcagagccacc

SEQ ID NO: 746 - DNA, 35 bp, Artificial Sequence
  Description of Artificial Sequence Synthetic polynucleotide
aactttttattctgaaacatgtgagccaacgctca

SEQ ID NO: 747 - DNA, 35 bp, Artificial Sequence
  Description of Artificial Sequence Synthetic polynucleotide
caaaatcttagcgtttgccattccacaaacaaata

SEQ ID NO: 748 - DNA, 35 bp, Artificial Sequence
  Description of Artificial Sequence Synthetic polynucleotide
caacctaatacgtaatgccacagtcaatcataagg

SEQ ID NO: 749 - DNA, 32 bp, Artificial Sequence
  Description of Artificial Sequence Synthetic polynucleotide
gaaagcgtaagaatacgtcagaagataaaact

SEQ ID NO: 750 - DNA, 32 bp, Artificial Sequence
  Description of Artificial Sequence Synthetic polynucleotide
gtttcctgtgtgaaattgttgcgctcactgct

SEQ ID NO: 751 - DNA, 32 bp, Artificial Sequence
  Description of Artificial Sequence Synthetic polynucleotide
gcgcgagctgaaaaggtgataaagctaaatcc

SEQ ID NO: 752 - DNA, 32 bp, Artificial Sequence
  Description of Artificial Sequence Synthetic polynucleotide
gagatagggttgagtgttaaccatcacccagg

SEQ ID NO: 753 - DNA, 32 bp, Artificial Sequence
  Description of Artificial Sequence Synthetic polynucleotide
aattttttcacgttgaaagttgcgccgacaag

SEQ ID NO: 754 - DNA, 32 bp, Artificial Sequence
  Description of Artificial Sequence Synthetic polynucleotide
aatcagagcgggagctaactgtccatcacgat

SEQ ID NO: 755 - DNA, 32 bp, Artificial Sequence
  Description of Artificial Sequence Synthetic polynucleotide
cagtaccaggcggataagagaaccgccacctt

SEQ ID NO: 756 - DNA, 32 bp, Artificial Sequence
  Description of Artificial Sequence Synthetic polynucleotide
taatggtttgaaataccgttaccagtataact

SEQ ID NO: 757 - DNA, 32 bp, Artificial Sequence
  Description of Artificial Sequence Synthetic polynucleotide
ccgccaccctcagaaccgggccttgatatttt

SEQ ID NO: 758 - DNA, 32 bp, Artificial Sequence
  Description of Artificial Sequence Synthetic polynucleotide
tgacccccagcgattataaggcgcagacggag

SEQ ID NO: 759 - DNA, 19 bp, Artificial Sequence
  Description of Artificial Sequence Synthetic polynucleotide
tgacctcctggttggtgta

SEQ ID NO: 760 - DNA, 19 bp, Artificial Sequence
  Description of Artificial Sequence Synthetic polynucleotide
aaatcatttctccgaactc

SEQ ID NO: 761 - DNA, 27 bp, Artificial Sequence
  Description of Artificial Sequence Synthetic polynucleotide
cgccctggagtgactctatgataccga

SEQ ID NO: 762 - DNA, 27 bp, Artificial Sequence
  Description of Artificial Sequence Synthetic polynucleotide
taaccccgcttctaatctatttacgct

SEQ ID NO: 763 - DNA, 23 bp, Artificial Sequence
  Description of Artificial Sequence Synthetic polynucleotide
caaccttatgacaatgtcccgcc

SEQ ID NO: 764 - DNA, 23 bp, Artificial Sequence
  Description of Artificial Sequence Synthetic polynucleotide
aagtgtccttagtgctgaattgt

SEQ ID NO: 765 - DNA, 19 bp, Artificial Sequence
  Description of Artificial Sequence Synthetic polynucleotide
tgcgggcctcttcgctatt

SEQ ID NO: 766 - DNA, 19 bp, Artificial Sequence
  Description of Artificial Sequence Synthetic polynucleotide
tgttgggaagggcgatcgg

SEQ ID NO: 767 - DNA, 27 bp, Artificial Sequence
  Description of Artificial Sequence Synthetic polynucleotide
cgacgacagtatcggcctcaggaagat

SEQ ID NO: 768 - DNA, 27 bp, Artificial Sequence
  Description of Artificial Sequence Synthetic polynucleotide
aaccgtgcatctgccagtttgagggga

SEQ ID NO: 769 - DNA, 23 bp, Artificial Sequence
  Description of Artificial Sequence Synthetic polynucleotide
tcacgttggtgtagatgggcgca

SEQ ID NO: 770 - DNA, 23 bp, Artificial Sequence
  Description of Artificial Sequence Synthetic polynucleotide
cggattgaccgtaatgggatagg

SEQ ID NO: 771 - DNA, 19 bp, Artificial Sequence
  Description of Artificial Sequence Synthetic polynucleotide
ttaaattgtaaacgttaat

SEQ ID NO: 772 - DNA, 19 bp, Artificial Sequence
  Description of Artificial Sequence Synthetic polynucleotide
agattgtataagcaaatat

SEQ ID NO: 773 - DNA, 27 bp, Artificial Sequence
  Description of Artificial Sequence Synthetic polynucleotide
gcctgagagtctggagcaaacaagaga

SEQ ID NO: 774 - DNA, 27 bp, Artificial Sequence
  Description of Artificial Sequence Synthetic polynucleotide
gagatctacaaaggctatcaggtcatt

SEQ ID NO: 775 - DNA, 23 bp, Artificial Sequence
  Description of Artificial Sequence Synthetic polynucleotide
aatgccggagagggtagctattt

SEQ ID NO: 776 - DNA, 23 bp, Artificial Sequence
  Description of Artificial Sequence Synthetic polynucleotide
tcaaccgttctagctgataaatt

SEQ ID NO: 777 - DNA, 19 bp, Artificial Sequence
  Description of Artificial Sequence Synthetic polynucleotide
aactccaacaggtcaggat

SEQ ID NO: 778 - DNA, 19 bp, Artificial Sequence
  Description of Artificial Sequence Synthetic polynucleotide
gcgaaccagaccggaagca

SEQ ID NO: 779 - DNA, 27 bp, Artificial Sequence
  Description of Artificial Sequence Synthetic polynucleotide
aggtctttaccctgactattatagtca

SEQ ID NO: 780 - DNA, 27 bp, Artificial Sequence
  Description of Artificial Sequence Synthetic polynucleotide
aacgagaatgaccataaatcaaaaatc

SEQ ID NO: 781 - DNA, 23 bp, Artificial Sequence
  Description of Artificial Sequence Synthetic polynucleotide
ccctcaaatgctttaaacagttc

SEQ ID NO: 782 - DNA, 23 bp, Artificial Sequence
  Description of Artificial Sequence Synthetic polynucleotide
cgtcataaatattcattgaatcc

SEQ ID NO: 783 - DNA, 19 bp, Artificial Sequence
  Description of Artificial Sequence Synthetic polynucleotide
aactaatgcagatacataa

SEQ ID NO: 784 - DNA, 19 bp, Artificial Sequence
  Description of Artificial Sequence Synthetic polynucleotide
atttaggaataccacattc

SEQ ID NO: 785 - DNA, 27 bp, Artificial Sequence
  Description of Artificial Sequence Synthetic polynucleotide
aagaactggctcattataccagtcagg

SEQ ID NO: 786 - DNA, 27 bp, Artificial Sequence
  Description of Artificial Sequence Synthetic polynucleotide
tcattgtgaattaccttatgcgatttt

SEQ ID NO: 787 - DNA, 23 bp, Artificial Sequence
  Description of Artificial Sequence Synthetic polynucleotide
cttgagatggtttaatttcaact

SEQ ID NO: 788 - DNA, 23 bp, Artificial Sequence
  Description of Artificial Sequence Synthetic polynucleotide
accagaacgagtagtaaattggg

SEQ ID NO: 789 - DNA, 19 bp, Artificial Sequence
  Description of Artificial Sequence Synthetic polynucleotide
ccgacttgagccatttggg

SEQ ID NO: 790 - DNA, 19 bp, Artificial Sequence
  Description of Artificial Sequence Synthetic polynucleotide
aggtgaattatcaccgtca

SEQ ID NO: 791 - DNA, 27 bp, Artificial Sequence
  Description of Artificial Sequence Synthetic polynucleotide
tcacaatcaatagaaaattcatatggt

SEQ ID NO: 792 - DNA, 27 bp, Artificial Sequence
  Description of Artificial Sequence Synthetic polynucleotide
aagacaccacggaataagtttattttg

SEQ ID NO: 793 - DNA, 23 bp, Artificial Sequence
  Description of Artificial Sequence Synthetic polynucleotide
aaggtggcaacatataaaagaaa

SEQ ID NO: 794 - DNA, 23 bp, Artificial Sequence
  Description of Artificial Sequence Synthetic polynucleotide
caaacgtagaaaatacatacata

SEQ ID NO: 795 - DNA, 19 bp, Artificial Sequence
  Description of Artificial Sequence Synthetic polynucleotide
taagcccaataataagagc

SEQ ID NO: 796 - DNA, 19 bp, Artificial Sequence
  Description of Artificial Sequence Synthetic polynucleotide
taacccacaagaattgagt

SEQ ID NO: 797 - DNA, 27 bp, Artificial Sequence
  Description of Artificial Sequence Synthetic polynucleotide
cctttacagagagaataacataaaaac

SEQ ID NO: 798 - DNA, 27 bp, Artificial Sequence
  Description of Artificial Sequence Synthetic polynucleotide
gtttaacgtcaaaaatgaaaatagcag

SEQ ID NO: 799 - DNA, 23 bp, Artificial Sequence
  Description of Artificial Sequence Synthetic polynucleotide
cccaatccaaataagaaacgatt

SEQ ID NO: 800 - DNA, 23 bp, Artificial Sequence
  Description of Artificial Sequence Synthetic polynucleotide
aaataaacagccatattatttat

SEQ ID NO: 801 - DNA, 19 bp, Artificial Sequence
  Description of Artificial Sequence Synthetic polynucleotide
cagatatagaaggcttatc

SEQ ID NO: 802 - DNA, 19 bp, Artificial Sequence
  Description of Artificial Sequence Synthetic polynucleotide
cgcccaatagcaagcaaat

SEQ ID NO: 803 - DNA, 27 bp, Artificial Sequence
  Description of Artificial Sequence Synthetic polynucleotide
taatcggctgtctttccttatcattcc

SEQ ID NO: 804 - DNA, 27 bp, Artificial Sequence
  Description of Artificial Sequence Synthetic polynucleotide
atttacgagcatgtagaaaccaatcaa

SEQ ID NO: 805 - DNA, 23 bp, Artificial Sequence
  Description of Artificial Sequence Synthetic polynucleotide
gaacaagaaaaataatatcccat

SEQ ID NO: 806 - DNA, 23 bp, Artificial Sequence
  Description of Artificial Sequence Synthetic polynucleotide
tttatcaacaatagataagtcct

SEQ ID NO: 807 - DNA, 19 bp, Artificial Sequence
  Description of Artificial Sequence Synthetic polynucleotide
ttgcttctgtaaatcgtcg

SEQ ID NO: 808 - DNA, 19 bp, Artificial Sequence
  Description of Artificial Sequence Synthetic polynucleotide
tatatgtgagtgaataacc

SEQ ID NO: 809 - DNA, 27 bp, Artificial Sequence
  Description of Artificial Sequence Synthetic polynucleotide
acctgagcaaaagaagatgatgaaaca

SEQ ID NO: 810 - DNA, 27 bp, Artificial Sequence
  Description of Artificial Sequence Synthetic polynucleotide
cgcagaggcgaattattcatttcaatt

SEQ ID NO: 811 - DNA, 23 bp, Artificial Sequence
  Description of Artificial Sequence Synthetic polynucleotide
tgctttgaataccaagttacaaa

SEQ ID NO: 812 - DNA, 23 bp, Artificial Sequence
  Description of Artificial Sequence Synthetic polynucleotide
aaacaataacggattcgcctgat

SEQ ID NO: 813 - DNA, 19 bp, Artificial Sequence
  Description of Artificial Sequence Synthetic polynucleotide
atcagatgatggcaattca

SEQ ID NO: 814 - DNA, 19 bp, Artificial Sequence
  Description of Artificial Sequence Synthetic polynucleotide
tatcatcatattcctgatt

SEQ ID NO: 815 - DNA, 27 bp, Artificial Sequence
  Description of Artificial Sequence Synthetic polynucleotide
caattcgacaactcgtattaaatcctt

SEQ ID NO: 816 - DNA, 27 bp, Artificial Sequence
  Description of Artificial Sequence Synthetic polynucleotide
ggatttagaagtattagactttacaaa

SEQ ID NO: 817 - DNA, 23 bp, Artificial Sequence
  Description of Artificial Sequence Synthetic polynucleotide
agagccgtcaatagataatacat

SEQ ID NO: 818 - DNA, 23 bp, Artificial Sequence
  Description of Artificial Sequence Synthetic polynucleotide
ggagcactaacaactaatagatt

SEQ ID NO: 819 - DNA, 20 bp, Artificial Sequence
  Description of Artificial Sequence Synthetic polynucleotide
atgacctcctggttggtgta

SEQ ID NO: 820 - DNA, 20 bp, Artificial Sequence
  Description of Artificial Sequence Synthetic polynucleotide
aaatcatttctccgaactca

SEQ ID NO: 821 - DNA, 28 bp, Artificial Sequence
  Description of Artificial Sequence Synthetic polynucleotide
acgccctggagtgactctatgataccga

SEQ ID NO: 822 - DNA, 28 bp, Artificial Sequence
  Description of Artificial Sequence Synthetic polynucleotide
taaccccgcttctaatctatttacgcta

SEQ ID NO: 823 - DNA, 24 bp, Artificial Sequence
  Description of Artificial Sequence Synthetic polynucleotide
acaaccttatgacaatgtcccgcc

SEQ ID NO: 824 - DNA, 24 bp, Artificial Sequence
  Description of Artificial Sequence Synthetic polynucleotide
aagtgtccttagtgctgaattgta

SEQ ID NO: 825 - DNA, 20 bp, Artificial Sequence
  Description of Artificial Sequence Synthetic polynucleotide
atgcgggcctcttcgctatt

SEQ ID NO: 826 - DNA, 20 bp, Artificial Sequence
  Description of Artificial Sequence Synthetic polynucleotide
tgttgggaagggcgatcgga

SEQ ID NO: 827 - DNA, 28 bp, Artificial Sequence
  Description of Artificial Sequence Synthetic polynucleotide
acgacgacagtatcggcctcaggaagat

SEQ ID NO: 828 - DNA, 28 bp, Artificial Sequence
  Description of Artificial Sequence Synthetic polynucleotide
aaccgtgcatctgccagtttgaggggaa

SEQ ID NO: 829 - DNA, 24 bp, Artificial Sequence
  Description of Artificial Sequence Synthetic polynucleotide
atcacgttggtgtagatgggcgca

SEQ ID NO: 830 - DNA, 24 bp, Artificial Sequence
  Description of Artificial Sequence Synthetic polynucleotide
cggattgaccgtaatgggatagga

SEQ ID NO: 831 - DNA, 20 bp, Artificial Sequence
  Description of Artificial Sequence Synthetic polynucleotide
attaaattgtaaacgttaat

SEQ ID NO: 832 - DNA, 20 bp, Artificial Sequence
  Description of Artificial Sequence Synthetic polynucleotide
agattgtataagcaaatata

SEQ ID NO: 833 - DNA, 28 bp, Artificial Sequence
  Description of Artificial Sequence Synthetic polynucleotide
agcctgagagtctggagcaaacaagaga

SEQ ID NO: 834 - DNA, 28 bp, Artificial Sequence
  Description of Artificial Sequence Synthetic polynucleotide
gagatctacaaaggctatcaggtcatta

SEQ ID NO: 835 - DNA, 24 bp, Artificial Sequence
  Description of Artificial Sequence Synthetic polynucleotide
aaatgccggagagggtagctattt

SEQ ID NO: 836 - DNA, 24 bp, Artificial Sequence
  Description of Artificial Sequence Synthetic polynucleotide
tcaaccgttctagctgataaatta

SEQ ID NO: 837 - DNA, 20 bp, Artificial Sequence
  Description of Artificial Sequence Synthetic polynucleotide
aaactccaacaggtcaggat

SEQ ID NO: 838 - DNA, 20 bp, Artificial Sequence
  Description of Artificial Sequence Synthetic polynucleotide
gcgaaccagaccggaagcaa

SEQ ID NO: 839 - DNA, 28 bp, Artificial Sequence
  Description of Artificial Sequence Synthetic polynucleotide
aaggtctttaccctgactattatagtca

SEQ ID NO: 840 - DNA, 28 bp, Artificial Sequence
  Description of Artificial Sequence Synthetic polynucleotide
aacgagaatgaccataaatcaaaaatca

SEQ ID NO: 841 - DNA, 24 bp, Artificial Sequence
  Description of Artificial Sequence Synthetic polynucleotide
accctcaaatgctttaaacagttc

SEQ ID NO: 842 - DNA, 24 bp, Artificial Sequence
  Description of Artificial Sequence Synthetic polynucleotide
cgtcataaatattcattgaatcca

SEQ ID NO: 843 - DNA, 20 bp, Artificial Sequence
  Description of Artificial Sequence Synthetic polynucleotide
aaactaatgcagatacataa

SEQ ID NO: 844 - DNA, 20 bp, Artificial Sequence
  Description of Artificial Sequence Synthetic polynucleotide
atttaggaataccacattca

SEQ ID NO: 845 - DNA, 28 bp, Artificial Sequence
  Description of Artificial Sequence Synthetic polynucleotide
aaagaactggctcattataccagtcagg

SEQ ID NO: 846 - DNA, 28 bp, Artificial Sequence
  Description of Artificial Sequence Synthetic polynucleotide
tcattgtgaattaccttatgcgatttta

SEQ ID NO: 847 - DNA, 24 bp, Artificial Sequence
  Description of Artificial Sequence Synthetic polynucleotide
acttgagatggtttaatttcaact

SEQ ID NO: 848 - DNA, 24 bp, Artificial Sequence
  Description of Artificial Sequence Synthetic polynucleotide
accagaacgagtagtaaattggga

SEQ ID NO: 849 - DNA, 20 bp, Artificial Sequence
  Description of Artificial Sequence Synthetic polynucleotide
accgacttgagccatttggg

SEQ ID NO: 850 - DNA, 20 bp, Artificial Sequence
  Description of Artificial Sequence Synthetic polynucleotide
aggtgaattatcaccgtcaa

SEQ ID NO: 851 - DNA, 28 bp, Artificial Sequence
  Description of Artificial Sequence Synthetic polynucleotide
atcacaatcaatagaaaattcatatggt

SEQ ID NO: 852 - DNA, 28 bp, Artificial Sequence
  Description of Artificial Sequence Synthetic polynucleotide
aagacaccacggaataagtttattttga

SEQ ID NO: 853 - DNA, 24 bp, Artificial Sequence
  Description of Artificial Sequence Synthetic polynucleotide
aaaggtggcaacatataaaagaaa

SEQ ID NO: 854 - DNA, 24 bp, Artificial Sequence
  Description of Artificial Sequence Synthetic polynucleotide
caaacgtagaaaatacatacataa

SEQ ID NO: 855 - DNA, 20 bp, Artificial Sequence
  Description of Artificial Sequence Synthetic polynucleotide
ataagcccaataataagagc

SEQ ID NO: 856 - DNA, 20 bp, Artificial Sequence
  Description of Artificial Sequence Synthetic polynucleotide
taacccacaagaattgagta

SEQ ID NO: 857 - DNA, 28 bp, Artificial Sequence
  Description of Artificial Sequence Synthetic polynucleotide
acctttacagagagaataacataaaaac

SEQ ID NO: 858 - DNA, 28 bp, Artificial Sequence
  Description of Artificial Sequence Synthetic polynucleotide
gtttaacgtcaaaaatgaaaatagcaga

SEQ ID NO: 859 - DNA, 24 bp, Artificial Sequence
  Description of Artificial Sequence Synthetic polynucleotide
acccaatccaaataagaaacgatt

SEQ ID NO: 860 - DNA, 24 bp, Artificial Sequence
  Description of Artificial Sequence Synthetic polynucleotide
aaataaacagccatattatttata

SEQ ID NO: 861 - DNA, 20 bp, Artificial Sequence
  Description of Artificial Sequence Synthetic polynucleotide
acagatatagaaggcttatc

SEQ ID NO: 862 - DNA, 20 bp, Artificial Sequence
  Description of Artificial Sequence Synthetic polynucleotide
cgcccaatagcaagcaaata

SEQ ID NO: 863 - DNA, 28 bp, Artificial Sequence
  Description of Artificial Sequence Synthetic polynucleotide
ataatcggctgtctttccttatcattcc

SEQ ID NO: 864 - DNA, 28 bp, Artificial Sequence
  Description of Artificial Sequence Synthetic polynucleotide
atttacgagcatgtagaaaccaatcaaa

SEQ ID NO: 865 - DNA, 24 bp, Artificial Sequence
  Description of Artificial Sequence Synthetic polynucleotide
agaacaagaaaaataatatcccat

SEQ ID NO: 866 - DNA, 24 bp, Artificial Sequence
  Description of Artificial Sequence Synthetic polynucleotide
tttatcaacaatagataagtccta

SEQ ID NO: 867 - DNA, 20 bp, Artificial Sequence
  Description of Artificial Sequence Synthetic polynucleotide
attgcttctgtaaatcgtcg

SEQ ID NO: 868 - DNA, 20 bp, Artificial Sequence
  Description of Artificial Sequence Synthetic polynucleotide
tatatgtgagtgaataacca

SEQ ID NO: 869 - DNA, 28 bp, Artificial Sequence
  Description of Artificial Sequence Synthetic polynucleotide
aacctgagcaaaagaagatgatgaaaca

SEQ ID NO: 870 - DNA, 28 bp, Artificial Sequence
  Description of Artificial Sequence Synthetic polynucleotide
cgcagaggcgaattattcatttcaatta

SEQ ID NO: 871 - DNA, 24 bp, Artificial Sequence
  Description of Artificial Sequence Synthetic polynucleotide
atgctttgaataccaagttacaaa

SEQ ID NO: 872 - DNA, 24 bp, Artificial Sequence
  Description of Artificial Sequence Synthetic polynucleotide
aaacaataacggattcgcctgata

SEQ ID NO: 873 - DNA, 20 bp, Artificial Sequence
  Description of Artificial Sequence Synthetic polynucleotide
aatcagatgatggcaattca

SEQ ID NO: 874 - DNA, 20 bp, Artificial Sequence
  Description of Artificial Sequence Synthetic polynucleotide
tatcatcatattcctgatta

SEQ ID NO: 875 - DNA, 28 bp, Artificial Sequence
  Description of Artificial Sequence Synthetic polynucleotide
acaattcgacaactcgtattaaatcctt

SEQ ID NO: 876 - DNA, 28 bp, Artificial Sequence
  Description of Artificial Sequence Synthetic polynucleotide
ggatttagaagtattagactttacaaaa

SEQ ID NO: 877 - DNA, 24 bp, Artificial Sequence
  Description of Artificial Sequence Synthetic polynucleotide
aagagccgtcaatagataatacat

SEQ ID NO: 878 - DNA, 24 bp, Artificial Sequence
  Description of Artificial Sequence Synthetic polynucleotide
ggagcactaacaactaatagatta

SEQ ID NO: 879 - DNA, 27 bp, Artificial Sequence
  Description of Artificial Sequence Synthetic polynucleotide
aggtgttgacacctcctggttggtgta

SEQ ID NO: 880 - DNA, 39 bp, Artificial Sequence
  Description of Artificial Sequence Synthetic polynucleotide
aaatcatttctccgaactcaggcgaagcgacaacaccta

SEQ ID NO: 881 - DNA, 35 bp, Artificial Sequence
  Description of Artificial Sequence Synthetic polynucleotide
gtatatgcgcccctggagtgactctatgataccga

SEQ ID NO: 882 - DNA, 47 bp, Artificial Sequence
  Description of Artificial Sequence Synthetic polynucleotide
taaccccgcttctaatctatttacgctaagcgaagcaagcatataca

SEQ ID NO: 883 - DNA, 31 bp, Artificial Sequence
  Description of Artificial Sequence Synthetic polynucleotide
accgagaagtaccttatgacaatgtcccgcc

SEQ ID NO: 884 - DNA, 43 bp, Artificial Sequence
  Description of Artificial Sequence Synthetic polynucleotide
aagtgtccttagtgctgaattgtccgcgaagcccggtcttcaa

SEQ ID NO: 885 - DNA, 27 bp, Artificial Sequence
  Description of Artificial Sequence Synthetic polynucleotide
caggtttcaccgggcctcttcgctatt

SEQ ID NO: 886 - DNA, 39 bp, Artificial Sequence
  Description of Artificial Sequence Synthetic polynucleotide
tgttgggaagggcgatcgggcgcgaagccggaaacctga

SEQ ID NO: 887 - DNA, 35 bp, Artificial Sequence
  Description of Artificial Sequence Synthetic polynucleotide
agcagacagcacgacagtatcggcctcaggaagat

SEQ ID NO: 888 - DNA, 47 bp, Artificial Sequence
  Description of Artificial Sequence Synthetic polynucleotide
aaccgtgcatctgccagtttgaggggattgcgaagctttgtctgcta

SEQ ID NO: 889 - DNA, 31 bp, Artificial Sequence
  Description of Artificial Sequence Synthetic polynucleotide
tatgccctagacgttggtgtagatgggcgca

SEQ ID NO: 890 - DNA, 43 bp, Artificial Sequence
  Description of Artificial Sequence Synthetic polynucleotide
cggattgaccgtaatgggataggaagcgaagcaactatcacta

SEQ ID NO: 891 - DNA, 27 bp, Artificial Sequence
  Description of Artificial Sequence Synthetic polynucleotide
agcagacaaaaaattgtaaacgttaat

SEQ ID NO: 892 - DNA, 39 bp, Artificial Sequence
  Description of Artificial Sequence Synthetic polynucleotide
agattgtataagcaaatatcggcgaagcgctgtctgcta

SEQ ID NO: 893 - DNA, 35 bp, Artificial Sequence
  Description of Artificial Sequence Synthetic polynucleotide
caggtttccgctgagagtctggagcaaacaagaga

SEQ ID NO: 894 - DNA, 47 bp, Artificial Sequence
  Description of Artificial Sequence Synthetic polynucleotide
gagatctacaaaggctatcaggtcatttggcgaagcgtgaaacctga

SEQ ID NO: 895 - DNA, 31 bp, Artificial Sequence
  Description of Artificial Sequence Synthetic polynucleotide
agtgatagtttgccggagagggtagctattt

SEQ ID NO: 896 - DNA, 43 bp, Artificial Sequence
  Description of Artificial Sequence Synthetic polynucleotide
tcaaccgttctagctgataaatttcgcgaagcctagggcataa

SEQ ID NO: 897 - DNA, 27 bp, Artificial Sequence
  Description of Artificial Sequence Synthetic polynucleotide
gtatatgcttctccaacaggtcaggat

SEQ ID NO: 898 - DNA, 39 bp, Artificial Sequence
  Description of Artificial Sequence Synthetic polynucleotide
gcgaaccagaccggaagcacggcgaagcgcgcatataca

SEQ ID NO: 899 - DNA, 35 bp, Artificial Sequence
  Description of Artificial Sequence Synthetic polynucleotide
aggtgttgtcgtctttaccctgactattatagtca

SEQ ID NO: 900 - DNA, 47 bp, Artificial Sequence
  Description of Artificial Sequence Synthetic polynucleotide
aacgagaatgaccataaatcaaaaatctggcgaagcgtcaacaccta

SEQ ID NO: 901 - DNA, 31 bp, Artificial Sequence
  Description of Artificial Sequence Synthetic polynucleotide
tgaagaccggctcaaatgctttaaacagttc

SEQ ID NO: 902 - DNA, 43 bp, Artificial Sequence
  Description of Artificial Sequence Synthetic polynucleotide
cgtcataaatattcattgaatcccagcgaagcacttctcggta

SEQ ID NO: 903 - DNA, 27 bp, Artificial Sequence
  Description of Artificial Sequence Synthetic polynucleotide
catgtacattctaatgcagatacataa

SEQ ID NO: 904 - DNA, 39 bp, Artificial Sequence
  Description of Artificial Sequence Synthetic polynucleotide
atttaggaataccacattccagcgaagcactgtacatga

SEQ ID NO: 905 - DNA, 35 bp, Artificial Sequence
  Description of Artificial Sequence Synthetic polynucleotide
gctgtattttgaactggctcattataccagtcagg

SEQ ID NO: 906 - DNA, 47 bp, Artificial Sequence
  Description of Artificial Sequence Synthetic polynucleotide
tcattgtgaattaccttatgcgattttatgcgaagctaaatacagca

SEQ ID NO: 907 - DNA, 31 bp, Artificial Sequence
  Description of Artificial Sequence Synthetic polynucleotide
gatagctagatgagatggtttaatttcaact

SEQ ID NO: 908 - DNA, 43 bp, Artificial Sequence
  Description of Artificial Sequence Synthetic polynucleotide
accagaacgagtagtaaattgggaggcgaagcgaacggttaga

SEQ ID NO: 909 - DNA, 27 bp, Artificial Sequence
  Description of Artificial Sequence Synthetic polynucleotide
tcctgtatgggacttgagccatttggg

SEQ ID NO: 910 - DNA, 39 bp, Artificial Sequence
  Description of Artificial Sequence Synthetic polynucleotide
aggtgaattatcaccgtcaacgcgaagccaatacaggaa

SEQ ID NO: 911 - DNA, 35 bp, Artificial Sequence
  Description of Artificial Sequence Synthetic polynucleotide
atgatgggagacaatcaatagaaaattcatatggt

SEQ ID NO: 912 - DNA, 47 bp, Artificial Sequence
  Description of Artificial Sequence Synthetic polynucleotide
aagacaccacggaataagtttattttgttgcgaagcttcccatcata

SEQ ID NO: 913 - DNA, 31 bp, Artificial Sequence
  Description of Artificial Sequence Synthetic polynucleotide
acgctatattggtggcaacatataaaagaaa

SEQ ID NO: 914 - DNA, 43 bp, Artificial Sequence
  Description of Artificial Sequence Synthetic polynucleotide
caaacgtagaaaatacatacatatggcgaagcgtaaccgagta

SEQ ID NO: 915 - DNA, 27 bp, Artificial Sequence
  Description of Artificial Sequence Synthetic polynucleotide
agcatctgatagcccaataataagagc

SEQ ID NO: 916 - DNA, 39 bp, Artificial Sequence
  Description of Artificial Sequence Synthetic polynucleotide
taacccacaagaattgagttagcgaagcatcagatgcta

SEQ ID NO: 917 - DNA, 35 bp, Artificial Sequence
  Description of Artificial Sequence Synthetic polynucleotide
ttacgtcgggtttacagagagaataacataaaaac

SEQ ID NO: 918 - DNA, 47 bp, Artificial Sequence
  Description of Artificial Sequence Synthetic polynucleotide
gtttaacgtcaaaaatgaaaatagcagcagcgaagcaccgacgtaaa

SEQ ID NO: 919 - DNA, 31 bp, Artificial Sequence
  Description of Artificial Sequence Synthetic polynucleotide
gtgcgaaaggcaatccaaataagaaacgatt

SEQ ID NO: 920 - DNA, 43 bp, Artificial Sequence
  Description of Artificial Sequence Synthetic polynucleotide
aaataaacagccatattatttatgagcgaagcagcatttgtca

SEQ ID NO: 921 - DNA, 27 bp, Artificial Sequence
  Description of Artificial Sequence Synthetic polynucleotide
ttacgtcggtgatatagaaggcttatc

SEQ ID NO: 922 - DNA, 39 bp, Artificial Sequence
  Description of Artificial Sequence Synthetic polynucleotide
cgcccaatagcaagcaaatccgcgaagccccgacgtaaa

SEQ ID NO: 923 - DNA, 35 bp, Artificial Sequence
  Description of Artificial Sequence Synthetic polynucleotide
agcatctgatatcggctgtctttccttatcattcc

SEQ ID NO: 924 - DNA, 47 bp, Artificial Sequence
  Description of Artificial Sequence Synthetic polynucleotide
atttacgagcatgtagaaaccaatcaatagcgaagcatcagatgcta

SEQ ID NO: 925 - DNA, 31 bp, Artificial Sequence
  Description of Artificial Sequence Synthetic polynucleotide
gacaaatgctacaagaaaaataatatcccat

SEQ ID NO: 926 - DNA, 43 bp, Artificial Sequence
  Description of Artificial Sequence Synthetic polynucleotide
tttatcaacaatagataagtcctccgcgaagccctttcgcaca

SEQ ID NO: 927 - DNA, 27 bp, Artificial Sequence
  Description of Artificial Sequence Synthetic polynucleotide
atgatgggaagcttctgtaaatcgtcg

SEQ ID NO: 928 - DNA, 39 bp, Artificial Sequence
  Description of Artificial Sequence Synthetic polynucleotide
tatatgtgagtgaataacctcgcgaagcctcccatcata

SEQ ID NO: 929 - DNA, 35 bp, Artificial Sequence
  Description of Artificial Sequence Synthetic polynucleotide
tcctgtattgctgagcaaaagaagatgatgaaaca

SEQ ID NO: 930 - DNA, 47 bp, Artificial Sequence
  Description of Artificial Sequence Synthetic polynucleotide
cgcagaggcgaattattcatttcaattccgcgaagcccatacaggaa

SEQ ID NO: 931 - DNA, 31 bp, Artificial Sequence
  Description of Artificial Sequence Synthetic polynucleotide
actcggttacctttgaataccaagttacaaa

SEQ ID NO: 932 - DNA, 43 bp, Artificial Sequence
  Description of Artificial Sequence Synthetic polynucleotide
aaacaataacggattcgcctgataagcgaagcaatatagcgta

SEQ ID NO: 933 - DNA, 27 bp, Artificial Sequence
  Description of Artificial Sequence Synthetic polynucleotide
gctgtatttacagatgatggcaattca

SEQ ID NO: 934 - DNA, 39 bp, Artificial Sequence
  Description of Artificial Sequence Synthetic polynucleotide
tatcatcatattcctgattaagcgaagcaaaatacagca

SEQ ID NO: 935 - DNA, 35 bp, Artificial Sequence
  Description of Artificial Sequence Synthetic polynucleotide
catgtacagtattcgacaactcgtattaaatcctt

SEQ ID NO: 936 - DNA, 47 bp, Artificial Sequence
  Description of Artificial Sequence Synthetic polynucleotide
ggatttagaagtattagactttacaaaaagcgaagcaatgtacatga

SEQ ID NO: 937 - DNA, 31 bp, Artificial Sequence
  Description of Artificial Sequence Synthetic polynucleotide
ctaaccgttcagccgtcaatagataatacat

SEQ ID NO: 938 - DNA, 43 bp, Artificial Sequence
  Description of Artificial Sequence Synthetic polynucleotide
ggagcactaacaactaatagattctgcgaagctctagctatca

SEQ ID NO: 939 - DNA, 27 bp, Artificial Sequence
  Description of Artificial Sequence Synthetic polynucleotide
cttggtgaacacctcctggttggtgta

SEQ ID NO: 940 - DNA, 39 bp, Artificial Sequence
  Description of Artificial Sequence Synthetic polynucleotide
aaatcatttctccgaactcaggcgaagcgatcaccaaga

SEQ ID NO: 941 - DNA, 35 bp, Artificial Sequence
  Description of Artificial Sequence Synthetic polynucleotide
gaaggatcgcccctggagtgactctatgataccga

SEQ ID NO: 942 - DNA, 47 bp, Artificial Sequence
  Description of Artificial Sequence Synthetic polynucleotide
taaccccgcttctaatctatttacgctaagcgaagcaagatccttca

SEQ ID NO: 943 - DNA, 31 bp, Artificial Sequence
  Description of Artificial Sequence Synthetic polynucleotide
atcccttggtaccttatgacaatgtcccgcc

SEQ ID NO: 944 - DNA, 43 bp, Artificial Sequence
  Description of Artificial Sequence Synthetic polynucleotide
aagtgtccttagtgctgaattgtccgcgaagcccgcattcgaa

SEQ ID NO: 945 - DNA, 27 bp, Artificial Sequence
  Description of Artificial Sequence Synthetic polynucleotide
accgttgtaccgggcctcttcgctatt

SEQ ID NO: 946 - DNA, 39 bp, Artificial Sequence
  Description of Artificial Sequence Synthetic polynucleotide
tgttgggaagggcgatcgggcgcgaagccgacaacggta

SEQ ID NO: 947 - DNA, 35 bp, Artificial Sequence
  Description of Artificial Sequence Synthetic polynucleotide
gttgaaaggcacgacagtatcggcctcaggaagat

SEQ ID NO: 948 - DNA, 47 bp, Artificial Sequence
  Description of Artificial Sequence Synthetic polynucleotide
aaccgtgcatctgccagtttgaggggattgcgaagcttctttcaaca

SEQ ID NO: 949 - DNA, 31 bp, Artificial Sequence
  Description of Artificial Sequence Synthetic polynucleotide
caccagaaagacgttggtgtagatgggcgca

SEQ ID NO: 950 - DNA, 43 bp, Artificial Sequence
  Description of Artificial Sequence Synthetic polynucleotide
cggattgaccgtaatgggataggaagcgaagcaaggctataaa

SEQ ID NO: 951 - DNA, 27 bp, Artificial Sequence
  Description of Artificial Sequence Synthetic polynucleotide
gttgaaagaaaaattgtaaacgttaat

SEQ ID NO: 952 - DNA, 39 bp, Artificial Sequence
  Description of Artificial Sequence Synthetic polynucleotide
agattgtataagcaaatatcggcgaagcgcctttcaaca

SEQ ID NO: 953 - DNA, 35 bp, Artificial Sequence
  Description of Artificial Sequence Synthetic polynucleotide
accgttgtcgctgagagtctggagcaaacaagaga

SEQ ID NO: 954 - DNA, 47 bp, Artificial Sequence
  Description of Artificial Sequence Synthetic polynucleotide
gagatctacaaaggctatcaggtcatttggcgaagcgtacaacggta

SEQ ID NO: 955 - DNA, 31 bp, Artificial Sequence
  Description of Artificial Sequence Synthetic polynucleotide
ttatagcctttgccggagagggtagctattt

SEQ ID NO: 956 - DNA, 43 bp, Artificial Sequence
  Description of Artificial Sequence Synthetic polynucleotide
tcaaccgttctagctgataaatttcgcgaagcctttctggtga

SEQ ID NO: 957 - DNA, 27 bp, Artificial Sequence
  Description of Artificial Sequence Synthetic polynucleotide
gaaggatcttctccaacaggtcaggat

SEQ ID NO: 958 - DNA, 39 bp, Artificial Sequence
  Description of Artificial Sequence Synthetic polynucleotide
gcgaaccagaccggaagcacggcgaagcgcgatccttca

SEQ ID NO: 959 - DNA, 35 bp, Artificial Sequence
  Description of Artificial Sequence Synthetic polynucleotide
cttggtgatcgtctttaccctgactattatagtca

SEQ ID NO: 960 - DNA, 47 bp, Artificial Sequence
  Description of Artificial Sequence Synthetic polynucleotide
aacgagaatgaccataaatcaaaaatctggcgaagcgttcaccaaga

SEQ ID NO: 961 - DNA, 31 bp, Artificial Sequence
  Description of Artificial Sequence Synthetic polynucleotide
tcgaatgcggctcaaatgctttaaacagttc

SEQ ID NO: 962 - DNA, 43 bp, Artificial Sequence
  Description of Artificial Sequence Synthetic polynucleotide
cgtcataaatattcattgaatcccagcgaagcaccaagggata

SEQ ID NO: 963 - DNA, 27 bp, Artificial Sequence
  Description of Artificial Sequence Synthetic polynucleotide
tacctgtattctaatgcagatacataa

SEQ ID NO: 964 - DNA, 39 bp, Artificial Sequence
  Description of Artificial Sequence Synthetic polynucleotide
atttaggaataccacattccagcgaagcactacaggtaa

SEQ ID NO: 965 - DNA, 35 bp, Artificial Sequence
  Description of Artificial Sequence Synthetic polynucleotide
taggtcaattgaactggctcattataccagtcagg

SEQ ID NO: 966 - DNA, 47 bp, Artificial Sequence
  Description of Artificial Sequence Synthetic polynucleotide
tcattgtgaattaccttatgcgattttatgcgaagctattgacctaa

SEQ ID NO: 967 - DNA, 31 bp, Artificial Sequence
  Description of Artificial Sequence Synthetic polynucleotide
aatgtggcgatgagatggtttaatttcaact

SEQ ID NO: 968 - DNA, 43 bp, Artificial Sequence
  Description of Artificial Sequence Synthetic polynucleotide
accagaacgagtagtaaattgggaggcgaagcgatgaatgaca

SEQ ID NO: 969 - DNA, 27 bp, Artificial Sequence
  Description of Artificial Sequence Synthetic polynucleotide
attggtctgggacttgagccatttggg

SEQ ID NO: 970 - DNA, 39 bp, Artificial Sequence
  Description of Artificial Sequence Synthetic polynucleotide
aggtgaattatcaccgtcaacgcgaagccaagaccaata

SEQ ID NO: 971 - DNA, 35 bp, Artificial Sequence
  Description of Artificial Sequence Synthetic polynucleotide
cactcaatagacaatcaatagaaaattcatatggt

SEQ ID NO: 972 - DNA, 47 bp, Artificial Sequence
  Description of Artificial Sequence Synthetic polynucleotide
aagacaccacggaataagtttattttgttgcgaagcttattgagtga

SEQ ID NO: 973 - DNA, 31 bp, Artificial Sequence
  Description of Artificial Sequence Synthetic polynucleotide
gaaatgccttggtggcaacatataaaagaaa

SEQ ID NO: 974 - DNA, 43 bp, Artificial Sequence
  Description of Artificial Sequence Synthetic polynucleotide
caaacgtagaaaatacatacatatggcgaagcgtgttctgtga

SEQ ID NO: 975 - DNA, 27 bp, Artificial Sequence
  Description of Artificial Sequence Synthetic polynucleotide
tgttgaccatagcccaataataagagc

SEQ ID NO: 976 - DNA, 39 bp, Artificial Sequence
  Description of Artificial Sequence Synthetic polynucleotide
taacccacaagaattgagttagcgaagcatggtcaacaa

SEQ ID NO: 977 - DNA, 35 bp, Artificial Sequence
  Description of Artificial Sequence Synthetic polynucleotide
tgactttcggtttacagagagaataacataaaaac

SEQ ID NO: 978 - DNA, 47 bp, Artificial Sequence
  Description of Artificial Sequence Synthetic polynucleotide
gtttaacgtcaaaaatgaaaatagcagcagcgaagcacgaaagtcaa

SEQ ID NO: 979 - DNA, 31 bp, Artificial Sequence
  Description of Artificial Sequence Synthetic polynucleotide
agagagtcggcaatccaaataagaaacgatt

SEQ ID NO: 980 - DNA, 43 bp, Artificial Sequence
  Description of Artificial Sequence Synthetic polynucleotide
aaataaacagccatattatttatgagcgaagcagtctacttca

SEQ ID NO: 981 - DNA, 27 bp, Artificial Sequence
  Description of Artificial Sequence Synthetic polynucleotide
tgactttcgtgatatagaaggcttatc

SEQ ID NO: 982 - DNA, 39 bp, Artificial Sequence
  Description of Artificial Sequence Synthetic polynucleotide
cgcccaatagcaagcaaatccgcgaagcccgaaagtcaa

SEQ ID NO: 983 - DNA, 35 bp, Artificial Sequence
  Description of Artificial Sequence Synthetic polynucleotide
tgttgaccatatcggctgtctttccttatcattcc

SEQ ID NO: 984 - DNA, 47 bp, Artificial Sequence
  Description of Artificial Sequence Synthetic polynucleotide
atttacgagcatgtagaaaccaatcaatagcgaagcatggtcaacaa

SEQ ID NO: 985 - DNA, 31 bp, Artificial Sequence
  Description of Artificial Sequence Synthetic polynucleotide
gaagtagactacaagaaaaataatatcccat

SEQ ID NO: 986 - DNA, 43 bp, Artificial Sequence
  Description of Artificial Sequence Synthetic polynucleotide
tttatcaacaatagataagtcctccgcgaagcccgactctcta

SEQ ID NO: 987 - DNA, 27 bp, Artificial Sequence
  Description of Artificial Sequence Synthetic polynucleotide
cactcaataagcttctgtaaatcgtcg

SEQ ID NO: 988 - DNA, 39 bp, Artificial Sequence
  Description of Artificial Sequence Synthetic polynucleotide
tatatgtgagtgaataacctcgcgaagcctattgagtga

SEQ ID NO: 989 - DNA, 35 bp, Artificial Sequence
  Description of Artificial Sequence Synthetic polynucleotide
attggtcttgctgagcaaaagaagatgatgaaaca

SEQ ID NO: 990 - DNA, 47 bp, Artificial Sequence
  Description of Artificial Sequence Synthetic polynucleotide
cgcagaggcgaattattcatttcaattccgcgaagcccagaccaata

SEQ ID NO: 991 - DNA, 31 bp, Artificial Sequence
  Description of Artificial Sequence Synthetic polynucleotide
cacagaacacctttgaataccaagttacaaa

SEQ ID NO: 992 - DNA, 43 bp, Artificial Sequence
  Description of Artificial Sequence Synthetic polynucleotide
aaacaataacggattcgcctgataagcgaagcaaggcatttca

SEQ ID NO: 993 - DNA, 27 bp, Artificial Sequence
  Description of Artificial Sequence Synthetic polynucleotide
taggtcaatacagatgatggcaattca

SEQ ID NO: 994 - DNA, 39 bp, Artificial Sequence
  Description of Artificial Sequence Synthetic polynucleotide
tatcatcatattcctgattaagcgaagcaattgacctaa

SEQ ID NO: 995 - DNA, 35 bp, Artificial Sequence
  Description of Artificial Sequence Synthetic polynucleotide
tacctgtagtattcgacaactcgtattaaatcctt

SEQ ID NO: 996 - DNA, 47 bp, Artificial Sequence
  Description of Artificial Sequence Synthetic polynucleotide
ggatttagaagtattagactttacaaaaagcgaagcaatacaggtaa

SEQ ID NO: 997 - DNA, 31 bp, Artificial Sequence
  Description of Artificial Sequence Synthetic polynucleotide
gtcattcatcagccgtcaatagataatacat

SEQ ID NO: 998 - DNA, 43 bp, Artificial Sequence
  Description of Artificial Sequence Synthetic polynucleotide
ggagcactaacaactaatagattctgcgaagctcgccacatta

SEQ ID NO: 999 - DNA, 27 bp, Artificial Sequence
  Description of Artificial Sequence Synthetic polynucleotide
tgcttggtacacctcctggttggtgta

SEQ ID NO: 1000 - DNA, 39 bp, Artificial Sequence
  Description of Artificial Sequence Synthetic polynucleotide
aaatcatttctccgaactcaggcgaagcgaaccaagcaa

SEQ ID NO: 1001 - DNA, 35 bp, Artificial Sequence
  Description of Artificial Sequence Synthetic polynucleotide
ccacttttgcccctggagtgactctatgataccga

SEQ ID NO: 1002 - DNA, 47 bp, Artificial Sequence
  Description of Artificial Sequence Synthetic polynucleotide
taaccccgcttctaatctatttacgctaagcgaagcaaaaaagtgga

SEQ ID NO: 1003 - DNA, 31 bp, Artificial Sequence
  Description of Artificial Sequence Synthetic polynucleotide
tcagcattgtaccttatgacaatgtcccgcc

SEQ ID NO: 1004 - DNA, 43 bp, Artificial Sequence
  Description of Artificial Sequence Synthetic polynucleotide
aagtgtccttagtgctgaattgtccgcgaagcccgatttccaa

SEQ ID NO: 1005 - DNA, 27 bp, Artificial Sequence
  Description of Artificial Sequence Synthetic polynucleotide
tgtgagaaaccgggcctcttcgctatt

SEQ ID NO: 1006 - DNA, 39 bp, Artificial Sequence
  Description of Artificial Sequence Synthetic polynucleotide
tgttgggaagggcgatcgggcgcgaagccgttctcacaa

SEQ ID NO: 1007 - DNA, 35 bp, Artificial Sequence
  Description of Artificial Sequence Synthetic polynucleotide
cggaaacagcacgacagtatcggcctcaggaagat

SEQ ID NO: 1008 - DNA, 47 bp, Artificial Sequence
  Description of Artificial Sequence Synthetic polynucleotide
aaccgtgcatctgccagtttgaggggattgcgaagctttgtttccga

SEQ ID NO: 1009 - DNA, 31 bp, Artificial Sequence
  Description of Artificial Sequence Synthetic polynucleotide
ttcctctgagacgttggtgtagatgggcgca

SEQ ID NO: 1010 - DNA, 43 bp, Artificial Sequence
  Description of Artificial Sequence Synthetic polynucleotide
cggattgaccgtaatgggataggaagcgaagcaacgctagtaa

SEQ ID NO: 1011 - DNA, 27 bp, Artificial Sequence
  Description of Artificial Sequence Synthetic polynucleotide
cggaaacaaaaaattgtaaacgttaat

SEQ ID NO: 1012 - DNA, 39 bp, Artificial Sequence
  Description of Artificial Sequence Synthetic polynucleotide
agattgtataagcaaatatcggcgaagcgctgtttccga

SEQ ID NO: 1013 - DNA, 35 bp, Artificial Sequence
  Description of Artificial Sequence Synthetic polynucleotide
tgtgagaacgctgagagtctggagcaaacaagaga

SEQ ID NO: 1014 - DNA, 47 bp, Artificial Sequence
  Description of Artificial Sequence Synthetic polynucleotide
gagatctacaaaggctatcaggtcatttggcgaagcgtttctcacaa

SEQ ID NO: 1015 - DNA, 31 bp, Artificial Sequence
  Description of Artificial Sequence Synthetic polynucleotide
tactagcgtttgccggagagggtagctattt

SEQ ID NO: 1016 - DNA, 43 bp, Artificial Sequence
  Description of Artificial Sequence Synthetic polynucleotide
tcaaccgttctagctgataaatttcgcgaagcctcagaggaaa

SEQ ID NO: 1017 - DNA, 27 bp, Artificial Sequence
  Description of Artificial Sequence Synthetic polynucleotide
ccacttttttctccaacaggtcaggat

SEQ ID NO: 1018 - DNA, 39 bp, Artificial Sequence
  Description of Artificial Sequence Synthetic polynucleotide
gcgaaccagaccggaagcacggcgaagcgcaaaagtgga

SEQ ID NO: 1019 - DNA, 35 bp, Artificial Sequence
  Description of Artificial Sequence Synthetic polynucleotide
tgcttggttcgtctttaccctgactattatagtca

SEQ ID NO: 1020 - DNA, 47 bp, Artificial Sequence
  Description of Artificial Sequence Synthetic polynucleotide
aacgagaatgaccataaatcaaaaatctggcgaagcgtaccaagcaa

SEQ ID NO: 1021 - DNA, 31 bp, Artificial Sequence
  Description of Artificial Sequence Synthetic polynucleotide
tggaaatcggctcaaatgctttaaacagttc

SEQ ID NO: 1022 - DNA, 43 bp, Artificial Sequence
  Description of Artificial Sequence Synthetic polynucleotide
cgtcataaatattcattgaatcccagcgaagcacaatgctgaa

SEQ ID NO: 1023 - DNA, 27 bp, Artificial Sequence
  Description of Artificial Sequence Synthetic polynucleotide
taacacacttctaatgcagatacataa

SEQ ID NO: 1024 - DNA, 39 bp, Artificial Sequence
  Description of Artificial Sequence Synthetic polynucleotide
atttaggaataccacattccagcgaagcacgtgtgttaa

SEQ ID NO: 1025 - DNA, 35 bp, Artificial Sequence
  Description of Artificial Sequence Synthetic polynucleotide
actttgccttgaactggctcattataccagtcagg

SEQ ID NO: 1026 - DNA, 47 bp, Artificial Sequence
  Description of Artificial Sequence Synthetic polynucleotide
tcattgtgaattaccttatgcgattttatgcgaagctaggcaaagta

SEQ ID NO: 1027 - DNA, 31 bp, Artificial Sequence
  Description of Artificial Sequence Synthetic polynucleotide
tcaaatcggatgagatggtttaatttcaact

SEQ ID NO: 1028 - DNA, 43 bp, Artificial Sequence
  Description of Artificial Sequence Synthetic polynucleotide
accagaacgagtagtaaattgggaggcgaagcgaagtcgtgaa

SEQ ID NO: 1029 - DNA, 27 bp, Artificial Sequence
  Description of Artificial Sequence Synthetic polynucleotide
tcgaaccagggacttgagccatttggg

SEQ ID NO: 1030 - DNA, 39 bp, Artificial Sequence
  Description of Artificial Sequence Synthetic polynucleotide
aggtgaattatcaccgtcaacgcgaagccatggttcgaa

SEQ ID NO: 1031 - DNA, 35 bp, Artificial Sequence
  Description of Artificial Sequence Synthetic polynucleotide
ttacggacagacaatcaatagaaaattcatatggt

SEQ ID NO: 1032 - DNA, 47 bp, Artificial Sequence
  Description of Artificial Sequence Synthetic polynucleotide
aagacaccacggaataagtttattttgttgcgaagcttgtccgtaaa

SEQ ID NO: 1033 - DNA, 31 bp, Artificial Sequence
  Description of Artificial Sequence Synthetic polynucleotide
gcgtttttttggtggcaacatataaaagaaa

SEQ ID NO: 1034 - DNA, 43 bp, Artificial Sequence
  Description of Artificial Sequence Synthetic polynucleotide
caaacgtagaaaatacatacatatggcgaagcgtagcgtagaa

SEQ ID NO: 1035 - DNA, 27 bp, Artificial Sequence
  Description of Artificial Sequence Synthetic polynucleotide
tcattcagatagcccaataataagagc

SEQ ID NO: 1036 - DNA, 39 bp, Artificial Sequence
  Description of Artificial Sequence Synthetic polynucleotide
taacccacaagaattgagttagcgaagcatctgaatgaa

SEQ ID NO: 1037 - DNA, 35 bp, Artificial Sequence
  Description of Artificial Sequence Synthetic polynucleotide
ttcatagcggtttacagagagaataacataaaaac

SEQ ID NO: 1038 - DNA, 47 bp, Artificial Sequence
  Description of Artificial Sequence Synthetic polynucleotide
gtttaacgtcaaaaatgaaaatagcagcagcgaagcacgctatgaaa

SEQ ID NO: 1039 - DNA, 31 bp, Artificial Sequence
  Description of Artificial Sequence Synthetic polynucleotide
ctgactagggcaatccaaataagaaacgatt

SEQ ID NO: 1040 - DNA, 43 bp, Artificial Sequence
  Description of Artificial Sequence Synthetic polynucleotide
aaataaacagccatattatttatgagcgaagcagtgttgctta

SEQ ID NO: 1041 - DNA, 27 bp, Artificial Sequence
  Description of Artificial Sequence Synthetic polynucleotide
ttcatagcgtgatatagaaggcttatc

SEQ ID NO: 1042 - DNA, 39 bp, Artificial Sequence
  Description of Artificial Sequence Synthetic polynucleotide
cgcccaatagcaagcaaatccgcgaagcccgctatgaaa

SEQ ID NO: 1043 - DNA, 35 bp, Artificial Sequence
  Description of Artificial Sequence Synthetic polynucleotide
tcattcagatatcggctgtctttccttatcattcc

SEQ ID NO: 1044 - DNA, 47 bp, Artificial Sequence
  Description of Artificial Sequence Synthetic polynucleotide
atttacgagcatgtagaaaccaatcaatagcgaagcatctgaatgaa

SEQ ID NO: 1045 - DNA, 31 bp, Artificial Sequence
  Description of Artificial Sequence Synthetic polynucleotide
aagcaacactacaagaaaaataatatcccat

SEQ ID NO: 1046 - DNA, 43 bp, Artificial Sequence
  Description of Artificial Sequence Synthetic polynucleotide
tttatcaacaatagataagtcctccgcgaagcccctagtcaga

SEQ ID NO: 1047 - DNA, 27 bp, Artificial Sequence
  Description of Artificial Sequence Synthetic polynucleotide
ttacggacaagcttctgtaaatcgtcg

SEQ ID NO: 1048 - DNA, 39 bp, Artificial Sequence
  Description of Artificial Sequence Synthetic polynucleotide
tatatgtgagtgaataacctcgcgaagcctgtccgtaaa

SEQ ID NO: 1049 - DNA, 35 bp, Artificial Sequence
  Description of Artificial Sequence Synthetic polynucleotide
tcgaaccatgctgagcaaaagaagatgatgaaaca

SEQ ID NO: 1050 - DNA, 47 bp, Artificial Sequence
  Description of Artificial Sequence Synthetic polynucleotide
cgcagaggcgaattattcatttcaattccgcgaagccctggttcgaa

SEQ ID NO: 1051 - DNA, 31 bp, Artificial Sequence
  Description of Artificial Sequence Synthetic polynucleotide
tctacgctacctttgaataccaagttacaaa

SEQ ID NO: 1052 - DNA, 43 bp, Artificial Sequence
  Description of Artificial Sequence Synthetic polynucleotide
aaacaataacggattcgcctgataagcgaagcaaaaaaacgca

SEQ ID NO: 1053 - DNA, 27 bp, Artificial Sequence
  Description of Artificial Sequence Synthetic polynucleotide
actttgcctacagatgatggcaattca

SEQ ID NO: 1054 - DNA, 39 bp, Artificial Sequence
  Description of Artificial Sequence Synthetic polynucleotide
tatcatcatattcctgattaagcgaagcaaggcaaagta

SEQ ID NO: 1055 - DNA, 35 bp, Artificial Sequence
  Description of Artificial Sequence Synthetic polynucleotide
taacacacgtattcgacaactcgtattaaatcctt

SEQ ID NO: 1056 - DNA, 47 bp, Artificial Sequence
  Description of Artificial Sequence Synthetic polynucleotide
ggatttagaagtattagactttacaaaaagcgaagcaagtgtgttaa

SEQ ID NO: 1057 - DNA, 31 bp, Artificial Sequence
  Description of Artificial Sequence Synthetic polynucleotide
tcacgacttcagccgtcaatagataatacat

SEQ ID NO: 1058 - DNA, 43 bp, Artificial Sequence
  Description of Artificial Sequence Synthetic polynucleotide
ggagcactaacaactaatagattctgcgaagctccgatttgaa

SEQ ID NO: 1059 - DNA, 7560 bp, M13 bacteriophage
agcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctccgtctttatcgaggtaacaagcaccacgtagcttaagccctgtttactcattacaccaaccaggaggtcagagttcggagaaatgatttatgtgaaatgcgtcagccgattcaaggcccctatattcgtgcccaccgacgagttgcttacagatggcagggccgcactgtcggtatcatagagtcactccagggcgagcgtaaatagattagaagcggggttattttggcgggacattgtcataaggttgacaattcagcactaaggacacttaagtcgtgcgcatgaattcacaaccacttagaagaacatccaccctggcttctcctgagaa

SEQ ID NO: 1060 - DNA, 42 bp, Artificial Sequence
  Description of Artificial Sequence Synthetic polynucleotide
acaaaaacgctcataggaggccgattaacctgagaagtgtcg

SEQ ID NO: 1061 - DNA, 42 bp, Artificial Sequence
  Description of Artificial Sequence Synthetic polynucleotide
cattgcaacaggataacaatattaccgcctttttataatcag

SEQ ID NO: 1062 - DNA, 42 bp, Artificial Sequence
  Description of Artificial Sequence Synthetic polynucleotide
ttagaatcagagcctataacgtgctttccatcaccagtcaca

SEQ ID NO: 1063 - DNA, 42 bp, Artificial Sequence
  Description of Artificial Sequence Synthetic polynucleotide
cgcgggagctaaacggaaatacctacatacattggcagatgc

SEQ ID NO: 1064 - DNA, 42 bp, Artificial Sequence
  Description of Artificial Sequence Synthetic polynucleotide
cgaccaggagcacggcgcttaatgcgccgtaaccactcaatc

SEQ ID NO: 1065 - DNA, 42 bp, Artificial Sequence
  Description of Artificial Sequence Synthetic polynucleotide
ctttgactaataaagaaatggattattttttgacgccacacc

SEQ ID NO: 1066 - DNA, 42 bp, Artificial Sequence
  Description of Artificial Sequence Synthetic polynucleotide
gtcttctgacctgacggtcacgctgcgcgctacagggcgcgg

SEQ ID NO: 1067 - DNA, 42 bp, Artificial Sequence
  Description of Artificial Sequence Synthetic polynucleotide
agagatagaaccctagggacattctggcgcgtactatggttg

SEQ ID NO: 1068 - DNA, 42 bp, Artificial Sequence
  Description of Artificial Sequence Synthetic polynucleotide
gcgctagggcgcccagaaagcgaaaggacacgaactgatagc

SEQ ID NO: 1069 - DNA, 42 bp, Artificial Sequence
  Description of Artificial Sequence Synthetic polynucleotide
cctggcaagtgtagaagcgtaagaatacgtctttaatgcgac

SEQ ID NO: 1070 - DNA, 42 bp, Artificial Sequence
  Description of Artificial Sequence Synthetic polynucleotide
cctaaaagaagggacgatttagagcttgaaccctaagacaat

SEQ ID NO: 1071 - DNA, 42 bp, Artificial Sequence
  Description of Artificial Sequence Synthetic polynucleotide
gagaaagcatcgccttgaatggctattagtggcacaagggag

SEQ ID NO: 1072 - DNA, 42 bp, Artificial Sequence
  Description of Artificial Sequence Synthetic polynucleotide
atagataaaacagaaagcactaaatcggacggggaaagccaa

SEQ ID NO: 1073 - DNA, 42 bp, Artificial Sequence
  Description of Artificial Sequence Synthetic polynucleotide
accaccagcagattattaaaaataccgatcggcgaacgtggc

SEQ ID NO: 1074 - DNA, 42 bp, Artificial Sequence
  Description of Artificial Sequence Synthetic polynucleotide
gttttttggggtacaaccatcacccaaaacaatgaaaaatct

SEQ ID NO: 1075 - DNA, 42 bp, Artificial Sequence
  Description of Artificial Sequence Synthetic polynucleotide
ggcgaggtgccgtaggtgaggcggtcaggagccagcagcaga

SEQ ID NO: 1076 - DNA, 42 bp, Artificial Sequence
  Description of Artificial Sequence Synthetic polynucleotide
aaagcatctacgtgtccaacgtcaaaggctattaaaccgcct

SEQ ID NO: 1077 - DNA, 42 bp, Artificial Sequence
  Description of Artificial Sequence Synthetic polynucleotide
tggcccacaccttgcagtgccacgctgatattaacagaacgt

SEQ ID NO: 1078 - DNA, 42 bp, Artificial Sequence
  Description of Artificial Sequence Synthetic polynucleotide
gctatctggtcagtggaacaagagtccagcgaaaaaccgtga

SEQ ID NO: 1079 - DNA, 42 bp, Artificial Sequence
  Description of Artificial Sequence Synthetic polynucleotide
accctcaatcaaaactgaacctcaaatagactatcagggcga

SEQ ID NO: 1080 - DNA, 42 bp, Artificial Sequence
  Description of Artificial Sequence Synthetic polynucleotide
tagggttgagtgagcaaaagaatagccctcctaacaactaat

SEQ ID NO: 1081 - DNA, 42 bp, Artificial Sequence
  Description of Artificial Sequence Synthetic polynucleotide
gcttgttccagttttggcaaatcaacagtctttaggagcaaa

SEQ ID NO: 1082 - DNA, 42 bp, Artificial Sequence
  Description of Artificial Sequence Synthetic polynucleotide
agattagtataaatgcgaaaatcctgttgctggttgaattga

SEQ ID NO: 1083 - DNA, 42 bp, Artificial Sequence
  Description of Artificial Sequence Synthetic polynucleotide
aatccctagccgtcggttatctaaaatattgaaagtgcccca

SEQ ID NO: 1084 - DNA, 42 bp, Artificial Sequence
  Description of Artificial Sequence Synthetic polynucleotide
ggattagactttacagcaagcggtccactgatggtggttctc

SEQ ID NO: 1085 - DNA, 42 bp, Artificial Sequence
  Description of Artificial Sequence Synthetic polynucleotide
ggatttagaagtaaaatagataatacatctcgaaatcggcaa

SEQ ID NO: 1086 - DNA, 42 bp, Artificial Sequence
  Description of Artificial Sequence Synthetic polynucleotide
accgcctggcccgcaacagctgattgcctttgagtaacatta

SEQ ID NO: 1087 - DNA, 42 bp, Artificial Sequence
  Description of Artificial Sequence Synthetic polynucleotide
agtgagagagttgcaaacaattcgacaaattttaaaagttga

SEQ ID NO: 1088 - DNA, 42 bp, Artificial Sequence
  Description of Artificial Sequence Synthetic polynucleotide
tcattttgacgggcggtttgcgtattggcaacgcgtaaatcc

SEQ ID NO: 1089 - DNA, 42 bp, Artificial Sequence
  Description of Artificial Sequence Synthetic polynucleotide
ccagtgagcggaaccccgaacgttattactcgtatcggggag

SEQ ID NO: 1090 - DNA, 42 bp, Artificial Sequence
  Description of Artificial Sequence Synthetic polynucleotide
ttatcatattcctgattaatgaatcggcgcgccagggtgggt

SEQ ID NO: 1091 - DNA, 42 bp, Artificial Sequence
  Description of Artificial Sequence Synthetic polynucleotide
agcggaattatctgaaagaaaccaccagcatttttcttttca

SEQ ID NO: 1092 - DNA, 42 bp, Artificial Sequence
  Description of Artificial Sequence Synthetic polynucleotide
cgggaaacctgtgtcactgcccgctttcaaataatggaaggg

SEQ ID NO: 1093 - DNA, 42 bp, Artificial Sequence
  Description of Artificial Sequence Synthetic polynucleotide
gtcgtgccagctgcattatcagatgatgttatacttctgagg

SEQ ID NO: 1094 - DNA, 42 bp, Artificial Sequence
  Description of Artificial Sequence Synthetic polynucleotide
ttagaacttgcgctaaagcctggggtgcgccggaaatcaata

SEQ ID NO: 1095 - DNA, 42 bp, Artificial Sequence
  Description of Artificial Sequence Synthetic polynucleotide
aattgcgctaccatcctgattgtttggagcaattcgcataaa

SEQ ID NO: 1096 - DNA, 42 bp, Artificial Sequence
  Description of Artificial Sequence Synthetic polynucleotide
taaaagaaattgcgcacacaacatacgactaatgagtgaggt

SEQ ID NO: 1097 - DNA, 42 bp, Artificial Sequence
  Description of Artificial Sequence Synthetic polynucleotide
taaaacagaaatatatcaaaattatttggtctaactcacatt

SEQ ID NO: 1098 - DNA, 42 bp, Artificial Sequence
  Description of Artificial Sequence Synthetic polynucleotide
gaaattgttatccccatagctgtttcctcagaaacaataacg

SEQ ID NO: 1099 - DNA, 42 bp, Artificial Sequence
  Description of Artificial Sequence Synthetic polynucleotide
tacgctcacaattctagattttcaggtttttacatcgggacg

SEQ ID NO: 1100 - DNA, 42 bp, Artificial Sequence
  Description of Artificial Sequence Synthetic polynucleotide
gattcgctcatggttcgataaagacggatacgtggagatgaa

SEQ ID NO: 1101 - DNA, 42 bp, Artificial Sequence
  Description of Artificial Sequence Synthetic polynucleotide
ttcgtaactgattgcagtaacagtaccttaacgtctgcttgt

SEQ ID NO: 1102 - DNA, 42 bp, Artificial Sequence
  Description of Artificial Sequence Synthetic polynucleotide
taaggcgaattattaacagggcttaagcggatccccgggttg

SEQ ID NO: 1103 - DNA, 42 bp, Artificial Sequence
  Description of Artificial Sequence Synthetic polynucleotide
aaaatcgcgcagtactttgaataccaagtcaccgagctcgaa

SEQ ID NO: 1104 - DNA, 42 bp, Artificial Sequence
  Description of Artificial Sequence Synthetic polynucleotide
acctcctggttgcgtcatttctccgaacttattacatttaac

SEQ ID NO: 1105 - DNA, 42 bp, Artificial Sequence
  Description of Artificial Sequence Synthetic polynucleotide
ctgtgtaatgagtacatttcaattacctaaaacaaaattaac

SEQ ID NO: 1106 - DNA, 42 bp, Artificial Sequence
  Description of Artificial Sequence Synthetic polynucleotide
aatttcaacataaatcggtgggcacgaaccatctgagaagat

SEQ ID NO: 1107 - DNA, 42 bp, Artificial Sequence
  Description of Artificial Sequence Synthetic polynucleotide
gcatttctttgaataaacaaacatcaaggagcaaataagcaa

SEQ ID NO: 1108 - DNA, 42 bp, Artificial Sequence
  Description of Artificial Sequence Synthetic polynucleotide
gacaatatatgtgaacagtgcggccctgtataggggccttcg

SEQ ID NO: 1109 - DNA, 42 bp, Artificial Sequence
  Description of Artificial Sequence Synthetic polynucleotide
cagtacataaattgtaccttttttaatgctgaatcggctgac

SEQ ID NO: 1110 - DNA, 42 bp, Artificial Sequence
  Description of Artificial Sequence Synthetic polynucleotide
ccctggagtgacctctaatctatttacggagaaaacatagcg

SEQ ID NO: 1111 - DNA, 42 bp, Artificial Sequence
  Description of Artificial Sequence Synthetic polynucleotide
tctctatgataccggtgaataaccttgccttagaatccttaa

SEQ ID NO: 1112 - DNA, 42 bp, Artificial Sequence
  Description of Artificial Sequence Synthetic polynucleotide
atagcttcccgctttagtgctgaattgtgcacgacaatcgtc

SEQ ID NO: 1113 - DNA, 42 bp, Artificial Sequence
  Description of Artificial Sequence Synthetic polynucleotide
aaataacagattaattaattaattttccttctgtattaagtg

SEQ ID NO: 1114 - DNA, 42 bp, Artificial Sequence
  Description of Artificial Sequence Synthetic polynucleotide
gccaaaatcataggttgtgaattcatgccaaccttatgacgc

SEQ ID NO: 1115 - DNA, 42 bp, Artificial Sequence
  Description of Artificial Sequence Synthetic polynucleotide
tagtgaatttattagacgctgagaagagaaaatgtcccgcca

SEQ ID NO: 1116 - DNA, 42 bp, Artificial Sequence
  Description of Artificial Sequence Synthetic polynucleotide
cagggtggatgtggagctttctcaggagtctgctgatgcaaa

SEQ ID NO: 1117 - DNA, 42 bp, Artificial Sequence
  Description of Artificial Sequence Synthetic polynucleotide
tttcttctaagtggtctgagagactaccactatatgtaaaaa

SEQ ID NO: 1118 - DNA, 42 bp, Artificial Sequence
  Description of Artificial Sequence Synthetic polynucleotide
tccaatcagtgccagtaacgccagggttgcaaggccctccgg

SEQ ID NO: 1119 - DNA, 42 bp, Artificial Sequence
  Description of Artificial Sequence Synthetic polynucleotide
gacggccgcaagacggttgggttatatatttttaagattaag

SEQ ID NO: 1120 - DNA, 42 bp, Artificial Sequence
  Description of Artificial Sequence Synthetic polynucleotide
ctttttagttaattaagggggatgtgctttcccagtcacgct

SEQ ID NO: 1121 - DNA, 42 bp, Artificial Sequence
  Description of Artificial Sequence Synthetic polynucleotide
ttttcaaatatataaaagaacgcgagaagcacgttgtaaaac

SEQ ID NO: 1122 - DNA, 42 bp, Artificial Sequence
  Description of Artificial Sequence Synthetic polynucleotide
cttcgctattacccggcgatcggtgcggaataaataagaata

SEQ ID NO: 1123 - DNA, 42 bp, Artificial Sequence
  Description of Artificial Sequence Synthetic polynucleotide
tggccagctggcgatcatcttctgaccttaaataaggcgtct

SEQ ID NO: 1124 - DNA, 42 bp, Artificial Sequence
  Description of Artificial Sequence Synthetic polynucleotide
aacaccgtgggaagggaaaccaggcaaaggcaccgatggttt

SEQ ID NO: 1125 - DNA, 42 bp, Artificial Sequence
  Description of Artificial Sequence Synthetic polynucleotide
caactgtgaatcattaccgaccgtgtgaaaatttacttctgg

SEQ ID NO: 1126 - DNA, 42 bp, Artificial Sequence
  Description of Artificial Sequence Synthetic polynucleotide
gaatgcgttatacaccagccagctttccgcgccattcgccag

SEQ ID NO: 1127 - DNA, 42 bp, Artificial Sequence
  Description of Artificial Sequence Synthetic polynucleotide
gtttagtatcataaaattactagaaaaaacattcaggctgcg

SEQ ID NO: 1128 - DNA, 42 bp, Artificial Sequence
  Description of Artificial Sequence Synthetic polynucleotide
tatcggcctcagtgttgaggggacgacggctttaacaacgcc

SEQ ID NO: 1129 - DNA, 42 bp, Artificial Sequence
  Description of Artificial Sequence Synthetic polynucleotide
aagaagatcgcactaattcttaccagtaagaatcgccatact

SEQ ID NO: 1130 - DNA, 42 bp, Artificial Sequence
  Description of Artificial Sequence Synthetic polynucleotide
aacatgttgccagtggataggtcacgttggcggataacgctc

SEQ ID NO: 1131 - DNA, 42 bp, Artificial Sequence
  Description of Artificial Sequence Synthetic polynucleotide
gtgcatcaatttaggtagggcttaattgtaaagcctgaccgt

SEQ ID NO: 1132 - DNA, 42 bp, Artificial Sequence
  Description of Artificial Sequence Synthetic polynucleotide
aaaatataaagtacccgtgggaacaaacggtgtagatgggga

SEQ ID NO: 1133 - DNA, 42 bp, Artificial Sequence
  Description of Artificial Sequence Synthetic polynucleotide
cagtaataagagcagcagaggcattttcgtcgcatcgtaacc

SEQ ID NO: 1134 - DNA, 42 bp, Artificial Sequence
  Description of Artificial Sequence Synthetic polynucleotide
gcgagtaacaaccacatcaacattaaatgatgcagaacgcgc

SEQ ID NO: 1135 - DNA, 42 bp, Artificial Sequence
  Description of Artificial Sequence Synthetic polynucleotide
acccgtcggattctcgacaaaaggtaaaatgttcagctaagc

SEQ ID NO: 1136 - DNA, 42 bp, Artificial Sequence
  Description of Artificial Sequence Synthetic polynucleotide
ctgtttacagctttataggaacgccatccagctcatgtccag

SEQ ID NO: 1137 - DNA, 42 bp, Artificial Sequence
  Description of Artificial Sequence Synthetic polynucleotide
ctgtagctcaacaacgacaataaacaacgtaattctttttta

SEQ ID NO: 1138 - DNA, 42 bp, Artificial Sequence
  Description of Artificial Sequence Synthetic polynucleotide
accccatcctaattaatttttgttaaataaaaataattcgtt

SEQ ID NO: 1139 - DNA, 42 bp, Artificial Sequence
  Description of Artificial Sequence Synthetic polynucleotide
gaaaaataatatgatagataagtcctgacgcgtctggccttc

SEQ ID NO: 1140 - DNA, 42 bp, Artificial Sequence
  Description of Artificial Sequence Synthetic polynucleotide
aatattttgttaacatttaaattgtaaaacggtattaaacca

SEQ ID NO: 1141 - DNA, 42 bp, Artificial Sequence
  Description of Artificial Sequence Synthetic polynucleotide
gtaaattcgcattatacgagcatgtagaattccaagaacgaa

SEQ ID NO: 1142 - DNA, 42 bp, Artificial Sequence
  Description of Artificial Sequence Synthetic polynucleotide
agtaccgagcaaatcccggttgataatccatgtcacaataat

SEQ ID NO: 1143 - DNA, 42 bp, Artificial Sequence
  Description of Artificial Sequence Synthetic polynucleotide
ttgtatacactcattgtctttccttatcaaccaatatcatat

SEQ ID NO: 1144 - DNA, 42 bp, Artificial Sequence
  Description of Artificial Sequence Synthetic polynucleotide
cgcgtaggaatcataatcgtaaaactagagaaaagccccaag

SEQ ID NO: 1145 - DNA, 42 bp, Artificial Sequence
  Description of Artificial Sequence Synthetic polynucleotide
ttttattttcatgccgagaacaagcaagggaaaacaggaaga

SEQ ID NO: 1146 - DNA, 42 bp, Artificial Sequence
  Description of Artificial Sequence Synthetic polynucleotide
caaacaagagaattttgcctgagagtctccgcgaggcgtttt

SEQ ID NO: 1147 - DNA, 42 bp, Artificial Sequence
  Description of Artificial Sequence Synthetic polynucleotide
aatcgatgaacggttaccgcgcccaatatattctaagaacgt

SEQ ID NO: 1148 - DNA, 42 bp, Artificial Sequence
  Description of Artificial Sequence Synthetic polynucleotide
agcgaaccaggtcaaatgccggagagggcgttctaaatcaga

SEQ ID NO: 1149 - DNA, 42 bp, Artificial Sequence
  Description of Artificial Sequence Synthetic polynucleotide
aggctatctcccgagaaggcttatccgggcaagcagctgata

SEQ ID NO: 1150 - DNA, 42 bp, Artificial Sequence
  Description of Artificial Sequence Synthetic polynucleotide
tagattagttgctaatatgatattcaactagctatttttggg

SEQ ID NO: 1151 - DNA, 42 bp, Artificial Sequence
  Description of Artificial Sequence Synthetic polynucleotide
gccttaaatcaatacttgcgggaggtttaaagagatctacaa

SEQ ID NO: 1152 - DNA, 42 bp, Artificial Sequence
  Description of Artificial Sequence Synthetic polynucleotide
ccggagacagtctttcaaaagggtgagatgagagcctaattt

SEQ ID NO: 1153 - DNA, 42 bp, Artificial Sequence
  Description of Artificial Sequence Synthetic polynucleotide
ttaaatcaccatcattttgcacccagctgagcgtctttccaa

SEQ ID NO: 1154 - DNA, 42 bp, Artificial Sequence
  Description of Artificial Sequence Synthetic polynucleotide
gccagtttaaagatagaaccctcatatagcaaggatatcctg

SEQ ID NO: 1155 - DNA, 42 bp, Artificial Sequence
  Description of Artificial Sequence Synthetic polynucleotide
gtgtaggacaaaatttaccaacgctaacacaattttaaaaat

SEQ ID NO: 1156 - DNA, 42 bp, Artificial Sequence
  Description of Artificial Sequence Synthetic polynucleotide
aataagaaacgattgcctttatttcaacttttaaatgcaaga

SEQ ID NO: 1157 - DNA, 42 bp, Artificial Sequence
  Description of Artificial Sequence Synthetic polynucleotide
tcccaatccaaatcaaacagccatattaattgcctgagtaat

SEQ ID NO: 1158 - DNA, 42 bp, Artificial Sequence
  Description of Artificial Sequence Synthetic polynucleotide
ccctgtaatactaagtaccaaaaacattttagggaagcgcat

SEQ ID NO: 1159 - DNA, 42 bp, Artificial Sequence
  Description of Artificial Sequence Synthetic polynucleotide
gctttgcgggagaattttgtttaacgtctaacataaaaacta

SEQ ID NO: 1160 - DNA, 42 bp, Artificial Sequence
  Description of Artificial Sequence Synthetic polynucleotide
tagacggatcggttagaattagcaaaatatcatacaaaatag

SEQ ID NO: 1161 - DNA, 42 bp, Artificial Sequence
  Description of Artificial Sequence Synthetic polynucleotide
aagctaagagaattctttacagagagaaaaaaatgaggcaag

SEQ ID NO: 1162 - DNA, 42 bp, Artificial Sequence
  Description of Artificial Sequence Synthetic polynucleotide
caaattgagcgctattaacatccaataataagcaataaagca

SEQ ID NO: 1163 - DNA, 42 bp, Artificial Sequence
  Description of Artificial Sequence Synthetic polynucleotide
aagtcagagggtgcaactgaacaccctgggcctcagagcata

SEQ ID NO: 1164 - DNA, 42 bp, Artificial Sequence
  Description of Artificial Sequence Synthetic polynucleotide
tcaattctactagacgagctgaaaaggtaaatgaaatagcaa

SEQ ID NO: 1165 - DNA, 42 bp, Artificial Sequence
  Description of Artificial Sequence Synthetic polynucleotide
taatagtagtagcaatatcagagagatagagcaagaaacaca

SEQ ID NO: 1166 - DNA, 42 bp, Artificial Sequence
  Description of Artificial Sequence Synthetic polynucleotide
tagctattggggcgtacatttcgcaaattttagttagaattg

SEQ ID NO: 1167 - DNA, 42 bp, Artificial Sequence
  Description of Artificial Sequence Synthetic polynucleotide
tttcattcttaccgaagcccaataataaacccacatgaccat

SEQ ID NO: 1168 - DNA, 42 bp, Artificial Sequence
  Description of Artificial Sequence Synthetic polynucleotide
agccgaacaaagtttgcgaacgagtagaggtcaataaccttc

SEQ ID NO: 1169 - DNA, 42 bp, Artificial Sequence
  Description of Artificial Sequence Synthetic polynucleotide
gtaagcagatagttaagccctttttaagatgtttagctatat

SEQ ID NO: 1170 - DNA, 42 bp, Artificial Sequence
  Description of Artificial Sequence Synthetic polynucleotide
catataacagtttgtgtctggaagtttcaattaagactcctt

SEQ ID NO: 1171 - DNA, 42 bp, Artificial Sequence
  Description of Artificial Sequence Synthetic polynucleotide
atgattcccaattcaccagaaggaaaccgaactggcatgaaa

SEQ ID NO: 1172 - DNA, 42 bp, Artificial Sequence
  Description of Artificial Sequence Synthetic polynucleotide
attacgcagtacggctgaatataatgcttggcttacgcaata

SEQ ID NO: 1173 - DNA, 42 bp, Artificial Sequence
  Description of Artificial Sequence Synthetic polynucleotide
caactaaagtatgtcggaatacccaaaagaggaaagagctta

SEQ ID NO: 1174 - DNA, 42 bp, Artificial Sequence
  Description of Artificial Sequence Synthetic polynucleotide
attggcaacatatagtcatttttgcggagtagctcaacatcc

SEQ ID NO: 1175 - DNA, 42 bp, Artificial Sequence
  Description of Artificial Sequence Synthetic polynucleotide
catacataaaggaatagcaaacgtagaatagttttaaatatg

SEQ ID NO: 1176 - DNA, 42 bp, Artificial Sequence
  Description of Artificial Sequence Synthetic polynucleotide
tttaattgctcccgtcaggattagagagaaatatggtttacc

SEQ ID NO: 1177 - DNA, 42 bp, Artificial Sequence
  Description of Artificial Sequence Synthetic polynucleotide
atttttgataagagaaagaaacgcaaagaatagaaaattcta

SEQ ID NO: 1178 - DNA, 42 bp, Artificial Sequence
  Description of Artificial Sequence Synthetic polynucleotide
agcgccacaacaggcgttttaattcgaggaaagacggaataa

SEQ ID NO: 1179 - DNA, 42 bp, Artificial Sequence
  Description of Artificial Sequence Synthetic polynucleotide
caaactcaagacaaattttgtcacaatcacaccacttcaaat

SEQ ID NO: 1180 - DNA, 42 bp, Artificial Sequence
  Description of Artificial Sequence Synthetic polynucleotide
gtaaggtaaatattttaagaggaagccccttcaaagcgaaaa

SEQ ID NO: 1181 - DNA, 42 bp, Artificial Sequence
  Description of Artificial Sequence Synthetic polynucleotide
attgagggagggttaagggcgacattcaagccagaccggaag

SEQ ID NO: 1182 - DNA, 42 bp, Artificial Sequence
  Description of Artificial Sequence Synthetic polynucleotide
gcaaagcggatttttgactattatagtcacattagagccagc

SEQ ID NO: 1183 - DNA, 42 bp, Artificial Sequence
  Description of Artificial Sequence Synthetic polynucleotide
ctgcatcaaaaagagacggaaattattcagccatttgggacg

SEQ ID NO: 1184 - DNA, 42 bp, Artificial Sequence
  Description of Artificial Sequence Synthetic polynucleotide
aaaatcatttacccaaacagttcagaaaatcccccgtgaatt

SEQ ID NO: 1185 - DNA, 42 bp, Artificial Sequence
  Description of Artificial Sequence Synthetic polynucleotide
tcaggtcccagtagccgtcaccgacttgattaaagtcaaatg

SEQ ID NO: 1186 - DNA, 42 bp, Artificial Sequence
  Description of Artificial Sequence Synthetic polynucleotide
atcaccaatgaaactaaatattcattgaacgagaatgaccta

SEQ ID NO: 1187 - DNA, 42 bp, Artificial Sequence
  Description of Artificial Sequence Synthetic polynucleotide
ggccggaaacgtcacaccattaccattagaataaatcaaaaa

SEQ ID NO: 1188 - DNA, 42 bp, Artificial Sequence
  Description of Artificial Sequence Synthetic polynucleotide
gcgtccaatactccaaatgtttagactggcctgtagcgcgtt

SEQ ID NO: 1189 - DNA, 42 bp, Artificial Sequence
  Description of Artificial Sequence Synthetic polynucleotide
aagcggaatcgtcacatcgatagcagcatttagcgtcagaaa

SEQ ID NO: 1190 - DNA, 42 bp, Artificial Sequence
  Description of Artificial Sequence Synthetic polynucleotide
ttcatcgaatagtaaaaatagcgagaggcagacgacagtagc

SEQ ID NO: 1191 - DNA, 42 bp, Artificial Sequence
  Description of Artificial Sequence Synthetic polynucleotide
agggggtgcattttgaatcaagtttgccccgtaatcgataaa

SEQ ID NO: 1192 - DNA, 42 bp, Artificial Sequence
  Description of Artificial Sequence Synthetic polynucleotide
gatcttttcataattaaccctcgtttaccttttgcaaaagga

SEQ ID NO: 1193 - DNA, 42 bp, Artificial Sequence
  Description of Artificial Sequence Synthetic polynucleotide
tagcgtttgccacacggtcatagcccccacaagttttgccag

SEQ ID NO: 1194 - DNA, 42 bp, Artificial Sequence
  Description of Artificial Sequence Synthetic polynucleotide
ggcatagtaagaagcgccaaaaggaattttagaaccgccacc

SEQ ID NO: 1195 - DNA, 42 bp, Artificial Sequence
  Description of Artificial Sequence Synthetic polynucleotide
aagcaacactatcacaaaatcaccggaagccgccaccctcat

SEQ ID NO: 1196 - DNA, 42 bp, Artificial Sequence
  Description of Artificial Sequence Synthetic polynucleotide
ctcagagtacataaattcatcagttgagattattacaccacc

SEQ ID NO: 1197 - DNA, 42 bp, Artificial Sequence
  Description of Artificial Sequence Synthetic polynucleotide
atgcagaccaccacccgcctccctcagaccagagccaggtag

SEQ ID NO: 1198 - DNA, 42 bp, Artificial Sequence
  Description of Artificial Sequence Synthetic polynucleotide
ggagccgccgccagactaacggaacaacatttaggaatacaa

SEQ ID NO: 1199 - DNA, 42 bp, Artificial Sequence
  Description of Artificial Sequence Synthetic polynucleotide
aaccaccaccagaacctcagagccgccaggcacattcaacta

SEQ ID NO: 1200 - DNA, 42 bp, Artificial Sequence
  Description of Artificial Sequence Synthetic polynucleotide
gaaaaatctacgaacagtcaggacgttgccatcctcattaaa

SEQ ID NO: 1201 - DNA, 42 bp, Artificial Sequence
  Description of Artificial Sequence Synthetic polynucleotide
tcttaataaaacgacattgacaggaggtacaaacaaataaag

SEQ ID NO: 1202 - DNA, 42 bp, Artificial Sequence
  Description of Artificial Sequence Synthetic polynucleotide
gccagaaattatacctttaatcattgtggagatggggtcaga

SEQ ID NO: 1203 - DNA, 42 bp, Artificial Sequence
  Description of Artificial Sequence Synthetic polynucleotide
ctggctctggaaagtggccttgatattctgaggcatttaatt

SEQ ID NO: 1204 - DNA, 42 bp, Artificial Sequence
  Description of Artificial Sequence Synthetic polynucleotide
cgcgtcatacatggagtaaattgggcttaattaccttatgcc

SEQ ID NO: 1205 - DNA, 42 bp, Artificial Sequence
  Description of Artificial Sequence Synthetic polynucleotide
cgttccagtaagatcgcagtctctgaattgcgattttaagaa

SEQ ID NO: 1206 - DNA, 42 bp, Artificial Sequence
  Description of Artificial Sequence Synthetic polynucleotide
ctgacgagaaacctcagtgaataaggctttaacagtgcccgt

SEQ ID NO: 1207 - DNA, 42 bp, Artificial Sequence
  Description of Artificial Sequence Synthetic polynucleotide
ataccagaacgagtcttttgatgatacaagtgccttgagtac

SEQ ID NO: 1208 - DNA, 42 bp, Artificial Sequence
  Description of Artificial Sequence Synthetic polynucleotide
ataaacagctcatttgacaagaaccggacttcatcactggta

SEQ ID NO: 1209 - DNA, 42 bp, Artificial Sequence
  Description of Artificial Sequence Synthetic polynucleotide
caaagctgttaatggttttaacggggtcggagtgtaagagta

SEQ ID NO: 1210 - DNA, 42 bp, Artificial Sequence
  Description of Artificial Sequence Synthetic polynucleotide
atgaaacatgaaagataggctggctgactattcattacccag

SEQ ID NO: 1211 - DNA, 42 bp, Artificial Sequence
  Description of Artificial Sequence Synthetic polynucleotide
acctattattctaaccccctgcctatttacaaatcaacgtaa

SEQ ID NO: 1212 - DNA, 42 bp, Artificial Sequence
  Description of Artificial Sequence Synthetic polynucleotide
atgaacggtgtattaactttgaaagaggcgaccaggcggata

SEQ ID NO: 1213 - DNA, 42 bp, Artificial Sequence
  Description of Artificial Sequence Synthetic polynucleotide
tgcagaccaggcgctattaagaggctgagttttgctcagtga

SEQ ID NO: 1214 - DNA, 42 bp, Artificial Sequence
  Description of Artificial Sequence Synthetic polynucleotide
agtgccgactgaccacttagccggaacgcgcgacccaagaga

SEQ ID NO: 1215 - DNA, 42 bp, Artificial Sequence
  Description of Artificial Sequence Synthetic polynucleotide
gaaccgatcgagagttaggattagcggggactccttgctcca

SEQ ID NO: 1216 - DNA, 42 bp, Artificial Sequence
  Description of Artificial Sequence Synthetic polynucleotide
agtatcaccgtactattgtgtcgaaatcaggcgcagacggcg

SEQ ID NO: 1217 - DNA, 42 bp, Artificial Sequence
  Description of Artificial Sequence Synthetic polynucleotide
ccggaataggtggaggttgatataagtaaatcaatcataagg

SEQ ID NO: 1218 - DNA, 42 bp, Artificial Sequence
  Description of Artificial Sequence Synthetic polynucleotide
gagatttgtatcgggcgaaacaaagtactagccaccaccctc

SEQ ID NO: 1219 - DNA, 42 bp, Artificial Sequence
  Description of Artificial Sequence Synthetic polynucleotide
gaatcgcctgataacaggaggtttagtagccaccctcagagc

SEQ ID NO: 1220 - DNA, 42 bp, Artificial Sequence
  Description of Artificial Sequence Synthetic polynucleotide
attttcaaccaagccaaaagaatacactaacctaacctcaga

SEQ ID NO: 1221 - DNA, 42 bp, Artificial Sequence
  Description of Artificial Sequence Synthetic polynucleotide
cgattatgggatagccaccctcagaaccccgccacaacgaaa

SEQ ID NO: 1222 - DNA, 42 bp, Artificial Sequence
  Description of Artificial Sequence Synthetic polynucleotide
acactgagtttcgtactacgaaggcaccaaaacactcatcat

SEQ ID NO: 1223 - DNA, 42 bp, Artificial Sequence
  Description of Artificial Sequence Synthetic polynucleotide
atgtaccgtaaccgcaagcccaataggacctttgacccccag

SEQ ID NO: 1224 - DNA, 42 bp, Artificial Sequence
  Description of Artificial Sequence Synthetic polynucleotide
taaacgggtaaagctcatgaggaagtttacacgatctaaagt

SEQ ID NO: 1225 - DNA, 42 bp, Artificial Sequence
  Description of Artificial Sequence Synthetic polynucleotide
caatacgtaatgcccaccagtacaaactatagttagcgtacc

SEQ ID NO: 1226 - DNA, 42 bp, Artificial Sequence
  Description of Artificial Sequence Synthetic polynucleotide
tttgtcggacttttatcggaacgagggttcagcagctgtagc

SEQ ID NO: 1227 - DNA, 42 bp, Artificial Sequence
  Description of Artificial Sequence Synthetic polynucleotide
gactaaatctttcccacagacagccctcacaacgccgaaaga

SEQ ID NO: 1228 - DNA, 42 bp, Artificial Sequence
  Description of Artificial Sequence Synthetic polynucleotide
atattttgctaaaccgggatcgtcacccagcaacggctactt

SEQ ID NO: 1229 - DNA, 42 bp, Artificial Sequence
  Description of Artificial Sequence Synthetic polynucleotide
tttctgtatgggtcagacgttagtaaatgcagaggctttgag

SEQ ID NO: 1230 - DNA, 42 bp, Artificial Sequence
  Description of Artificial Sequence Synthetic polynucleotide
gcagggagttaataattcggtcgctgaggataataatttttt

SEQ ID NO: 1231 - DNA, 42 bp, Artificial Sequence
  Description of Artificial Sequence Synthetic polynucleotide
gaaggccgcttttgaactttcaacagttaggaattgcgaaat

SEQ ID NO: 1232 - DNA, 42 bp, Artificial Sequence
  Description of Artificial Sequence Synthetic polynucleotide
cacgttgccgatatccgatagttgcgccttcttaaagtgaga

SEQ ID NO: 1233 - DNA, 42 bp, Artificial Sequence
  Description of Artificial Sequence Synthetic polynucleotide
cgcataaaaaatctaaaggaacaactaatcagcggacagctt

SEQ ID NO: 1234 - DNA, 32 bp, Artificial Sequence
  Description of Artificial Sequence Synthetic polynucleotide
cacgcaaattaaccaaaaaagagtctgtccat

SEQ ID NO: 1235 - DNA, 30 bp, Artificial Sequence
  Description of Artificial Sequence Synthetic polynucleotide
gggttgtagttttttttttttcaatacttc

SEQ ID NO: 1236 - DNA, 36 bp, Artificial Sequence
  Description of Artificial Sequence Synthetic polynucleotide
gaagaactcaaattttttttttttctatcggccttg

SEQ ID NO: 1237 - DNA, 42 bp, Artificial Sequence
  Description of Artificial Sequence Synthetic polynucleotide
ctggtaaaccgagtcggtacgccagaatagggattagtaata

SEQ ID NO: 1238 - DNA, 42 bp, Artificial Sequence
  Description of Artificial Sequence Synthetic polynucleotide
tgaggcctatccagcacttgcctgagtatttgattttagaca

SEQ ID NO: 1239 - DNA, 36 bp, Artificial Sequence
  Description of Artificial Sequence Synthetic polynucleotide
tgatggtggttcttttttttttttcgaaatcggcaa

SEQ ID NO: 1240 - DNA, 50 bp, Artificial Sequence
  Description of Artificial Sequence Synthetic polynucleotide
ggattagactttacaaacatttttttttttttgagagagttgcagcaagc

SEQ ID NO: 1241 - DNA, 36 bp, Artificial Sequence
  Description of Artificial Sequence Synthetic polynucleotide
ttgaggatttagaagtaaaatagataatacattttt

SEQ ID NO: 1242 - DNA, 28 bp, Artificial Sequence
  Description of Artificial Sequence Synthetic polynucleotide
cgcaaattaaccaacggtacgccagaat

SEQ ID NO: 1243 - DNA, 32 bp, Artificial Sequence
  Description of Artificial Sequence Synthetic polynucleotide
tgaggccaccgagtaaaagagtctgtccataa

SEQ ID NO: 1244 - DNA, 31 bp, Artificial Sequence
  Description of Artificial Sequence Synthetic polynucleotide
gtatatgcgtatacttctttgattagtaata

SEQ ID NO: 1245 - DNA, 44 bp, Artificial Sequence
  Description of Artificial Sequence Synthetic polynucleotide
agggattttagacagggttgtaggcggcgaagcccggcatatac

SEQ ID NO: 1246 - DNA, 34 bp, Artificial Sequence
  Description of Artificial Sequence Synthetic polynucleotide
accgagaagaatcggccttgctggtaatatccag

SEQ ID NO: 1247 - DNA, 47 bp, Artificial Sequence
  Description of Artificial Sequence Synthetic polynucleotide
cacttgcctgagtagaagaactcaaaaaggcgaagccaattctcggt

SEQ ID NO: 1248 - DNA, 31 bp, Artificial Sequence
  Description of Artificial Sequence Synthetic polynucleotide
ttgctttcgaggtgaatttcttaaacagctt

SEQ ID NO: 1249 - DNA, 35 bp, Artificial Sequence
  Description of Artificial Sequence Synthetic polynucleotide
tcagcggagtgagaatattgtatcggtttatcaca

SEQ ID NO: 1250 - DNA, 41 bp, Artificial Sequence
  Description of Artificial Sequence Synthetic polynucleotide
caggtttccgttgctttcgaggtgaatttcttaaacagctt

SEQ ID NO: 1251 - DNA, 54 bp, Artificial Sequence
  Description of Artificial Sequence Synthetic polynucleotide
tcagcggagtgagaatattgtatcggtttatcacaggcgaagccacgaaacctg

SEQ ID NO: 1252 - DNA, 38 bp, Artificial Sequence
  Description of Artificial Sequence Synthetic polynucleotide
agcagacattaaggagcctttaagaaaggaacaactaa

SEQ ID NO: 1253 - DNA, 51 bp, Artificial Sequence
  Description of Artificial Sequence Synthetic polynucleotide
cacgttgaaaatctccaaaaaaaaggctccctggcgaagcctctgtctgct

SEQ ID NO: 1254 - DNA, 21 bp, Artificial Sequence
  Description of Artificial Sequence Synthetic polynucleotide
atacttctttgattagtaata

SEQ ID NO: 1255 - DNA, 25 bp, Artificial Sequence
  Description of Artificial Sequence Synthetic polynucleotide
agggattttagacagggttgtaggc

SEQ ID NO: 1256 - DNA, 31 bp, Artificial Sequence
  Description of Artificial Sequence Synthetic polynucleotide
caggtttcgtatacttctttgattagtaata

SEQ ID NO: 1257 - DNA, 44 bp, Artificial Sequence
  Description of Artificial Sequence Synthetic polynucleotide
agggattttagacagggttgtaggcggcgaagcccggaaacctg

SEQ ID NO: 1258 - DNA, 34 bp, Artificial Sequence
  Description of Artificial Sequence Synthetic polynucleotide
agcagacagaatcggccttgctggtaatatccag

SEQ ID NO: 1259 - DNA, 47 bp, Artificial Sequence
  Description of Artificial Sequence Synthetic polynucleotide
cacttgcctgagtagaagaactcaaaaaggcgaagccaatgtctgct

SEQ ID NO: 1260 - DNA, 28 bp, Artificial Sequence
  Description of Artificial Sequence Synthetic polynucleotide
aaggagcctttaagaaaggaacaactaa

SEQ ID NO: 1261 - DNA, 32 bp, Artificial Sequence
  Description of Artificial Sequence Synthetic polynucleotide
cacgttgaaaatctccaaaaaaaaggctccct

SEQ ID NO: 1262 - DNA, 41 bp, Artificial Sequence
  Description of Artificial Sequence Synthetic polynucleotide
ctatcactcgttgctttcgaggtgaatttcttaaacagctt

SEQ ID NO: 1263 - DNA, 54 bp, Artificial Sequence
  Description of Artificial Sequence Synthetic polynucleotide
tcagcggagtgagaatattgtatcggtttatcacaggcgaagccacagtgatag

SEQ ID NO: 1264 - DNA, 38 bp, Artificial Sequence
  Description of Artificial Sequence Synthetic polynucleotide
catgtacattaaggagcctttaagaaaggaacaactaa

SEQ ID NO: 1265 - DNA, 51 bp, Artificial Sequence
  Description of Artificial Sequence Synthetic polynucleotide
cacgttgaaaatctccaaaaaaaaggctccctggcgaagcctctgtacatg

(19) United States
(12) Patent Application Publication
(10) Pub. No.: US 2010/0216978 A1
(43) Pub. Date: Aug. 26, 2010

(54) WIREFRAME NANOSTRUCTURES

(75) Inventor: William Shih, Cambridge, MA (US)

Correspondence Address: WOLF GREENFIELD & SACKS, P.C., 600 ATLANTIC AVENUE, BOSTON, MA, 02210-2206, US

(73) Assignee: Dsna-Farber Cancer Institute Inc., Bostom, MA (US)

(21) Appl. No.: 12/594,264

(22) PCT Filed: Apr. 17, 2008
(86) PCT No.: PCT/US2008/004939

§ 371 Date: May 3, 2010

Related U.S. Application Data
(60) Provisional application No. 60/923,831, filed on Apr. 17, 2007.

(51) Int. Cl. C07H 21/00

(52) U.S. Cl. 536/22.1; 977/700; 977/734

(57) ABSTRACT

The present invention generally relates to nanotechnology and, in particular, to wireframe nanostructures which may be formed from nucleic acids. In various aspects, the invention relates to molecular structures having a plurality of vertices and pathways connecting the vertices, which may be formed from nucleic acids, including bundles or tubes of nucleic acids. Such molecular structures may form shapes such as icosahedrons, octahedrons, tetrahedrons, or other polyhedra, which may define an interior space. The interior space may be used, for example, to contain a molecule for further study, or to contain a molecule for drug delivery purposes. In some cases, the molecular structure may be stabilized using relatively short nucleic acid strands that interact with two or more nucleic acid portions within the structure, thereby substantially immobilizing the portions relative to each other. Other aspects of the invention relate to techniques for forming such molecular structures, techniques for using such molecular structures, techniques of promoting such molecular structures, kits involving such molecular structures, and the like.

### RELATED APPLICATIONS

This application claims the benefit of U.S. Provisional Patent Application Ser. No. 60/923,831, filed Apr. 17, 2007, entitled “Wireframe Nanostructures,” by Shih, incorporated herein by reference.

### FIELD OF INVENTION

The present invention generally relates to nanotechnology and, in particular, to wireframe nanostructures which may be formed from nucleic acids.

### BACKGROUND

There have been recent efforts to produce molecular containers or cages that can contain other molecules, for example, to confine molecules, e.g., for NMR analysis or electron microscopy. An example of such a molecule is buckminsterfullerene, or C₆₀, which defines an interior space having a diameter of about 1 nm, large enough for atoms such as xenon. In addition, DNA has been used to produce molecular containers having the shape of cubes or truncated octahedra. However, such structures are not easily altered and have thus found only limited uses as molecular containers or cages.

### SUMMARY OF THE INVENTION

The present invention generally relates to nanotechnology and, in particular, to wireframe nanostructures which may be formed from nucleic acids. The subject matter of the present invention involves, in some cases, interrelated products, alternative solutions to a particular problem, and/or a plurality of different uses of one or more systems and/or articles.

One aspect of the invention is directed to a composition. The composition, according to one set of embodiments, comprises a molecular structure defining a plurality of vertices and pathways, at least some of the vertices having at least three pathways emanating therefrom, each of which connects two vertices. In one embodiment, at least one pathway connecting two vertices comprises a nanotube comprising nucleic acid. In another embodiment, at least one pathway connecting two vertices comprises a nucleic acid and/or has a length between the two vertices of at least about 40 nm. In yet another embodiment, at least one pathway connecting two vertices comprises a six-helix nucleic acid bundle and/or a ten-helix nucleic acid bundle.

The composition, in another set of embodiments, includes a molecular structure defining a three-dimensional interior space. In certain embodiments, the molecular structure is formed from one or more nucleic acids and/or is substantially rigid. In one embodiment, the molecular structure is formed from one or more nucleic acids having a persistence of at least about 100 nm.

According to yet another set of embodiments, the composition includes a molecular structure defining a three-dimensional interior space, where the molecular structure is formed from one or more nucleic acids and has a smallest dimension of at least about 100 nm.

Still another set of embodiments includes a composition comprising a substantially rigid molecular structure formed from nucleic acid, and a lipid membrane associated with at least a portion of the substantially rigid molecular structure. Yet another set of embodiments is directed to a liposome rigidified by a nucleic acid.

Another aspect of the invention is directed to a method. The method, in one set of embodiments, includes an act of administering, to a subject, a composition comprising a molecular structure defining an interior space and a plurality of vertices and pathways, at least some of the vertices having at least three pathways emanating therefrom, each of which connects two vertices. In one embodiment, at least one pathway connecting two vertices comprises a nanotube comprising nucleic acid. In another embodiment, at least one pathway connecting two vertices comprises a nucleic acid and has a length between the two vertices of at least about 40 nm. In yet another embodiment, at least one pathway connecting two vertices comprises a six-helix nucleic acid bundle. In still another embodiment, at least one pathway connecting two vertices comprises a ten-helix nucleic acid bundle.

The method, according to another set of embodiments, includes an act of administering, to a subject, a composition comprising a molecular structure defining a three-dimensional interior space. In one embodiment, the molecular structure is formed from one or more nucleic acids and/or is substantially rigid. In another embodiment, the molecular structure is formed from one or more nucleic acids having a persistence of at least about 100 nm. In yet another set of embodiments, the molecular structure is formed from one or more nucleic acids and having a smallest dimension of at least about 100 nm.

The method according to still another set of embodiments, includes an act of exposing a first molecular structure comprising a first nucleic acid to a second molecular structure comprising a second nucleic acid to produce a combined molecular structure defining a plurality of vertices and pathways, at least some of the vertices having at least three pathways emanating therefrom, each of which connects two vertices. In one embodiment, at least one pathway connecting two vertices comprises a nanotube. In another embodiment, at least one pathway connecting two vertices comprises a nanotube.

The method, in yet another set of embodiments, includes acts of preparing one or more structures that when folded, produces a polyhedron (or other structure, as described herein), the one or more structures comprising, prior to folding, one or more vertices and one or more pathways that connect two vertices and/or half-pathways that, when connected with other half-pathways when the one or more structures are folded, connects two vertices; preparing one or more routes that span the one or more vertices and the one or more pathways and/or half-pathways of the one or more structures; mapping one or more nucleic acids having a length of at least about 1,000 nucleotides on the one or more routes; and identifying a location within the one or more nucleic acids that are to be immobilized relative to each other when the structure is folded to produce the polyhedron. In some cases, at least one pair of vertices is connected by at least four spans of the one or more routes, for example, six or ten spans.

In another aspect, the present invention is directed to a method of making one or more of the embodiments described herein, for example, a molecular wireframe structure. In another aspect, the present invention is directed to a method of using one or more of the embodiments described herein, for example, a molecular wireframe structure.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1C illustrate various nucleic acid bundles, according to certain embodiments of the invention;

FIGS. 2A-2B illustrate the formation of a tetrahedral structure, according to one embodiment of the invention;

FIGS. 3A-3C illustrate the formation of nucleic acid nanotubes, according to another embodiment of the invention;

FIGS. 4A-4D illustrate the formation of icosahedral structures, in various embodiments of the invention;

FIGS. 5A-5D illustrate tetrahedra formed in one embodiment of the invention;

FIGS. 6A-6B illustrate icosahedra formed in another embodiment of the invention;

FIGS. 7A-7D illustrate various sequences used to produce a molecular wireframe structure, in accordance with one embodiment of the invention;

FIGS. 8A-8B illustrate various stabilizers used to produce a molecular wireframe structure, in another embodiment of the invention;

FIGS. 9A-9D illustrate a six-helix nucleic acid bundle stabilized using various stabilizers, according to yet another embodiment of the invention;

FIGS. 10A-10B illustrate the results of an experiment showing specific heterodimerization between two nucleic acids, in one embodiment of the invention; and

FIGS. 11A-11B illustrate the formation of nucleic acid nanotubes, according to another embodiment of the invention;

FIG. 12 illustrates a half-strut, in one embodiment of the invention;

FIGS. 13A-13E illustrate various sequences used to produce a molecular wireframe structure, in accordance with another embodiment of the invention; and

FIGS. 14A-14H illustrate various sequences used to produce a molecular wireframe structure, in accordance with another embodiment of the invention.

### DETAILED DESCRIPTION

The present invention generally relates to nanotechnology and, in particular, to wireframe nanostructures which may be formed from nucleic acids. In various aspects, the invention relates to molecular structures having a plurality of vertices and pathways connecting the vertices, which may be formed from nucleic acids, including bundles or tubes of nucleic acids. Such molecular structures may form shapes such as icosahedrons, octahedrons, tetrahedrons, or other polyhedra, which may define an interior space. The interior space may be used, for example, to contain a molecule for further study, or to contain a molecule for drug delivery purposes. In some cases, the molecular structure may be stabilized using relatively short nucleic acid strands that interact with two or more nucleic acid portions within the structure, thereby substantially immobilizing the portions relative to each other. Other aspects of the invention relate to techniques for forming such molecular structures, techniques for using such molecular structures, techniques of promoting such molecular structures, kits involving such molecular structures, and the like.

Various aspects of the present invention are generally directed to molecular structures that are typically supramolecular, i.e., portions of the molecular structure are held together by noncovalent bonds such as hydrogen bonding or hydrophobic forces, and/or effects such as physical entanglement. Thus, a molecular structure can include one, or a plurality of molecules. In such molecular structures, besides the molecules themselves (i.e., their primary structure), the three-dimensional relationship, or the positions of the molecules relative to the overall molecular structure, can also be important. In some embodiments of the present invention, the overall three-dimensional structure of the molecular structure itself is novel, irrespective of the primary structure of the molecules forming the molecular structure.

For example, in certain embodiments, as discussed below, molecular structures of the present invention include vertices and pathways connecting the vertices that together define a novel molecular shape, which may define an interior space in some cases. The interior space may be used, for example, to contain a molecule or a drug. An “interior space,” as used herein, is defined as a three-dimensional volume of space that is created by an at least partially enclosing molecular structure. Typically, in an interior space, one of the dimensions is not substantially smaller than the other dimensions, i.e., the interior space has a length, a width, and a thickness (and is not merely defined by a length and a width but substantially no thickness, such as would be created by a relatively flat, two-dimensional structure, such as a benzene ring or a two-dimensional circle of nucleic acid). The interior space may have any shape (which is created by the enclosing molecular structure), for example, spherical, ellipsoidal (prolate or oblate), cubical, icosahedral, etc. Often, the length, width, and thickness of the interior space are of comparable dimensions, and in some cases, the dimensions are substantially the same, for example, as in a spherical interior space, a cubic interior space, or an icosahedral interior space.

The molecular structure may be formed from one or more nucleic acids according to one aspect of the invention. As used herein, a “nucleic acid” is given its ordinary meaning as used in the art, and may include deoxyribonucleic acid (DNA), ribonucleic acid (RNA), and/or artificial nucleic acids, such as a peptide nucleic acid (PNA). The molecular structure may include one type of nucleic acid (e.g., DNA), or more than one type in some cases, which may form part of the same molecule and/or different molecules assembled together in a supramolecular assembly defining the overall molecular structure. Typically, the nucleic acid is a polymeric molecule comprising one or more “bases” (usually nitrogeneous) connected to a backbone structure, which may be a sugar-phosphate backbone (e.g., as in DNA or RNA) or a peptide backbone (e.g., as in PNA).

The sugars within the nucleic acid, when present, may be, for example, ribose sugars (as in RNA), or deoxyribose sugars (as in DNA). In some cases, the nucleic acid may comprise both ribose and deoxyribose sugars. Examples of bases that may be found within a nucleic acid include, but are not limited to, the naturally-occurring bases (e.g., adenosine or “A,” thymidine or “T,” guanosine or “G,” cytidine or “C,” or uridine or “U”). The bases typically interact on a specific basis (i.e., guanosine interacts with cytidine via hydrogen bonding and vice versa, and adenosine interacts with thymidine or uridine via hydrogen bonding and vice versa). In some cases, the nucleic acid may comprise nucleoside analogs (e.g., 2-aminoadenosine, 2-thiothymidine, inosine, pyrrolopyrimidine, 3-methyladenosine, C5-bromouridine, C5-fluorouridine, C5-iodouridine, C5-propynyluridine, C5-propynylcytidine, C5-methylcytidine, 7-deazaadenosine, 7-deazaguanosine, 8-oxoadenosine, 8-oxoguanosine, 06-methylguanosine, 2-thiocytidine, 2-aminopurine, 2-amino-6-chloropurine, 2,6-diaminopurine, hypoxanthine), chemically or biologically modified bases (e.g., methylated bases), intercalated bases, modified sugars (2′-fluororibose, arabinose, or hexose), modified phosphate moieties (e.g., phosphorothioates or 5′-N-phosphoramidite linkages), and/or other naturally and non-naturally occurring bases substitutable into the nucleic acid, including substituted and unsubstituted aromatic moieties. Other suitable base and/or backbone modifications are well-known to those of skill in the art.

The nucleic acid present within the molecular structure may be single-stranded or double-stranded, i.e., formed of two strands (or of the same strand looped back on itself, such as in a hairpin turn or a stem-loop structure) associated with each other via hydrogen bonding, e.g., via guanosine/cytidine base-pair interactions, adenosine/thymidine base-pair interactions, adenosine/uridine base-pair interactions, etc.

In certain embodiments of the invention, the nucleic acids may be present within the molecular structure as “bundles,” which may comprise two or more non-complementary nucleic acid portions associated with each other. The nucleic acids forming the bundles may be single stranded or double stranded, and the non-complementary nucleic acid portions may be part of the same nucleic acid molecule, or may be part of different nucleic acid molecules. For instance, there may be 2, 3, 4, 5, 6, 8, 10, 12, 14, 16, 18, 20, 24, 30, 42, 54, 66, 78, 90, or more non-complementary nucleic acid portions associated with each other as part of a bundle. In certain embodiments, there may be other nucleic acid strands associated with one or more portions of the nucleic acids forming the nucleic acid bundle, e.g., to provide stability, as discussed below.

It should be noted that, in a bundle of nucleic acid, not all of the nucleic acid strands need run from one end of the bundle to the other. For example, one or more nucleic acid strands may run from a first end of the bundle, through a hairpin turn or a stem-loop structure, back to the first end of the bundle (or may go through more than one hairpin turn or a stem-loop structure, in some cases); or a nucleic acid strand may end within the bundle.

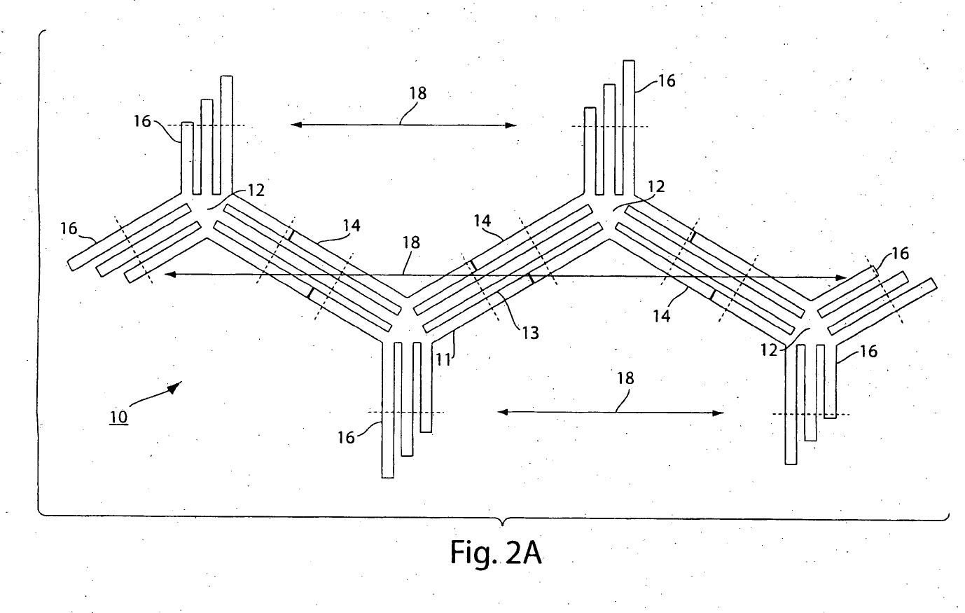

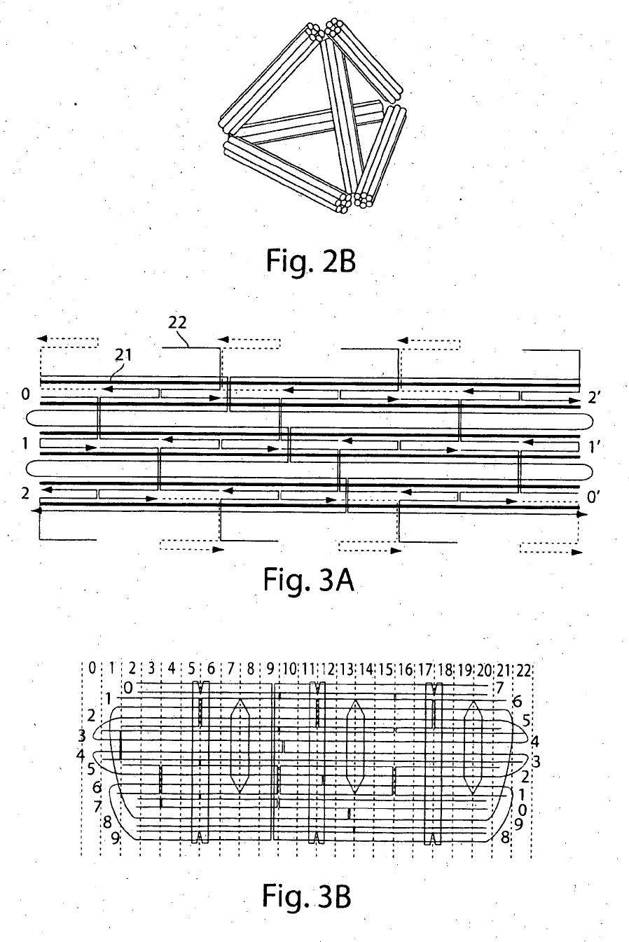

As a specific example, referring now to FIG. 2, a layout 10 in FIG. 2A of a nucleic acid that may be folded to form a tetrahedron is shown. The folded tetrahedron structure is shown in FIG. 2B. Each edge of the tetrahedron is formed from a six-helix nanotube. In the layout of FIG. 2A, there are four vertices 12, each having three pathways emanating therefrom. In some cases, the pathways extend directly between two vertices, such as pathway 14, and within each pathway, the nucleic acid strands run from one vertex to the other. For instance, in a pathway connecting two vertices 12, there may be six spans running between the two vertices (which, when assembled, may result in a six-helix nucleic acid bundle). In other cases, however, such as half-pathway 16, the nucleic acids do not run from one vertex to the other, but instead run from one vertex back to the same vertex. When the layout is assembled to form a tetrahedron, however, a complete pathway between two vertices is still formed, as nucleic acid stabilizers or “staples” (discussed below) can be used to connect the two half-pathways to form a complete pathway extending between two vertices in the final assembled molecular structure. In FIG. 2A, there are three such connections that need to be made between various half-pathways; these are indicated by arrows 18.

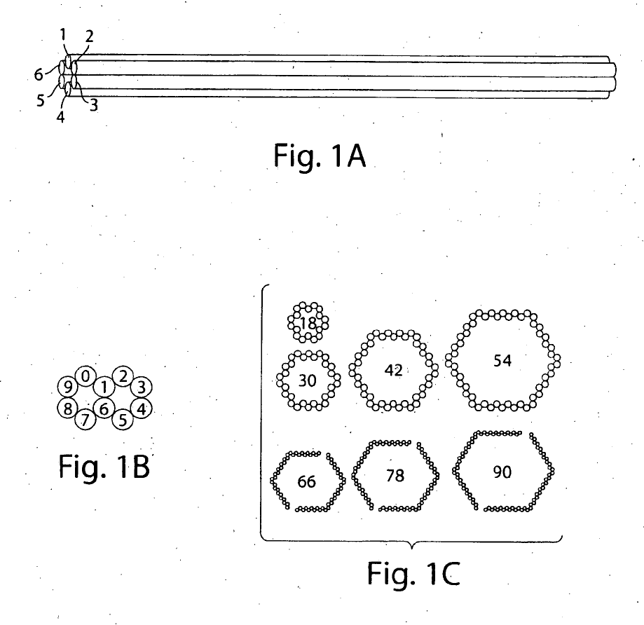

In some cases, the bundles may define a nanotube. The nanotube may have a hollow center, with nucleic acid strands arranged around the center (thus, a double strand of DNA, by itself, is not a nanotube, as the two sugar-phosphate backbones forming the DNA are interconnected by bases hydrogen bonded to each other, which thus does not result in a hollow center). The nanotube may be circular or elliptical, or in some cases, the nanotube may have polygonal shapes such as a hexagon. In some cases, the nanotube may have more than one hollow center, e.g., having the shape of a lemniscate. Non-limiting examples of such nanotubes are shown in FIG. 1A (with a six-helix nucleic acid bundle), FIG. 1B (with a ten-helix nucleic acid bundle, having a lemniscate shape with two, hollow centers; thus, more than one hollow center may be present within the nanotube), and FIG. 1C (with the number of nucleic acid strands present within the nanotube shown in the center of each nanotube). As noted, the nucleic acid portions forming the bundled nanotube may be part of the same nucleic acid molecule, and/or may be part of different nucleic acid molecules. In some cases, the nanotube may be formed from an even number of nucleic acid strands (e.g., 4, 6, 8, 10, 12, etc.). In certain embodiments, other molecules may be present within the nanotube, for example, to provide stability to the nanotube structure, as discussed below. See also Mathieu et al., “Six-Helix Bundles Designed from DNA,” Nano Letters 5, 661-665, 2005 for other examples of nanotubes.

In some embodiments, one or more of the nucleic acid bundles or nanotubes within the molecular structure may be fabricated from one or more relatively long nucleic acids, e.g., having lengths of at least about 500 nucleotides, at least about 1,000 nucleotides, at least about 3,000 nucleotides, at least about 10,000 nucleotides, at least about 30,000 nucleotides, etc. Such a nucleic acid may be referred to as a nucleic acid scaffold. The nucleic acid scaffold may form a single bundle or nanotube, or may comprise different parts of different bundles or nanotubes in the final molecular structure. For instance, a nucleic acid scaffold may wrap in various ways around the molecular structure, e.g., forming various nucleic acid bundles or nanotubes defining the molecular structure. In some cases, a nucleic acid may form a first portion of a nucleic acid bundle and a second portion of the same nucleic acid bundle (or a different one), where the first and second portions forming the nucleic acid bundle are not complementary. Non-limiting examples of such configurations are discussed below. In one set of embodiments, the nucleic acid scaffolds are substantially free of self-complementary regions and/or repeat units, as discussed below. In certain embodiments of the invention, the nucleic acid scaffolds are immobilized to form one or more bundles or nanotubes, and ultimately a three-dimensional structure, using one or more nucleic acid stabilizers able to associate with two or more portions of the nucleic acid.

One source of a nucleic acid having such characteristics is bacteriophage DNA, for example, M13 bacteriophage. The DNA in such bacteriophages may be single stranded DNA, and have substantially few self-complementary regions (e.g., only 2 hairpin regions may form), and a length on the order of 7,000 nucleotides. The DNA can be removed from the bacteriophage using DNA isolation techniques known to those of ordinary skill in the art, for example, by using lysis buffer (e.g., comprising an alkaline environment and/or surfactant) followed by centrifugation at greater than 10,000 RCF (relative centrifugal force) to separate the DNA.

The molecular structure may be stabilized, in some cases, by nucleic acid stabilizers able to associate with two or more nucleic acid portions. For example, a nucleic acid stabilizer may comprise a first portion complementary to a first nucleic acid strand (e.g., a nucleic acid scaffold) and a second portion complementary to a second nucleic acid strand. The first and second portions may be part of the same nucleic acid molecule, and/or may be part of different molecules. In some cases, the stabilizer may be formed essentially from nucleic acid. A nucleic acid stabilizer may have a length of between about 20 nucleotides and about 100 nucleotides, for example, between about 35 nucleotides and about 45 nucleotides, or about 40 nucleotides. As the first portion of the nucleic acid stabilizer binds to the first nucleic acid portion and the second portion binds to the second nucleic acid portions, the two portions are substantially immobilized, relative to each other, due to the present of the stabilizer. Thus, the two portions are not able to move apart, or at least are not able to move far apart, and remain associated together. By using a plurality of such nucleic acid stabilizers, e.g., targeted to different nucleic acids or different portions of nucleic acids, one or more nucleic acids can be stabilized in a substantially rigid configuration, e.g., as a bundle or a nanotube. In addition, as described above, these can further be configured as part of larger molecular structures, such as the wireframe molecular structures described above, and accordingly, a wireframe molecular structure may comprise a plurality of nucleic acid stabilizers that are used to hold the wireframe molecular structure together. An example of a technique for forming such nucleic acid stabilizers is illustrated in Rothemund, P. W. K., “Folding DNA to Create Nanoscale Shapes and Patterns,” Nature, 440:297-302 (2006).

In some embodiments, as discussed below, a plurality of stabilizers may be targeted to one or more nucleic acid scaffolds such that the stabilizers are not attracted to overlapping regions of the target nucleic acids, i.e., each stabilizer uniquely substantially immobilizes two nucleic acid scaffold portions together. Methods of producing such stabilizers are discussed below. This may be advantageous, for example, where one or more nucleic acid scaffolds are used to form a wireframe molecular structure, which structure is stabilized by the presence of the nucleic acid stabilizers. If the nucleic acid scaffolds are substantially free of self-complementary regions and/or repeat units, i.e., the nucleic acid scaffolds have relatively unique nucleic acid sequences, then a plurality of stabilizers may be targeted to unique, or at least specific, locations within each nucleic acid scaffold, which may thus allow the nucleic acid scaffold to form a specific wireframe molecular structure.

In another set of embodiments, two or more nucleic acid scaffold portions may be stabilized by the use of more than one stabilizer (although not all nucleic acid scaffold portions need be stabilized using more than one stabilizer; and in some cases, different portions may be stabilized using different numbers of stabilizers, which may be independently chosen in some cases). As above, the stabilizers may each be formed essentially from nucleic acid, and they may each independently have any suitable length, e.g., between about 20 nucleotides and about 100 nucleotides, for example, between about 35 nucleotides and about 45 nucleotides, or about 40 nucleotides, etc. In some cases, a first nucleic acid portion is stabilized to a second nucleic acid portion using a first stabilizer and a second stabilizer, where the first stabilizer contains a first portion substantially complementary to the first nucleic acid portion and a second portion substantially complementary to a portion of the second stabilizer, and the second stabilizer contains a first portion substantially complementary to the second nucleic acid portion and a second portion substantially complementary to a portion of the first stabilizer.

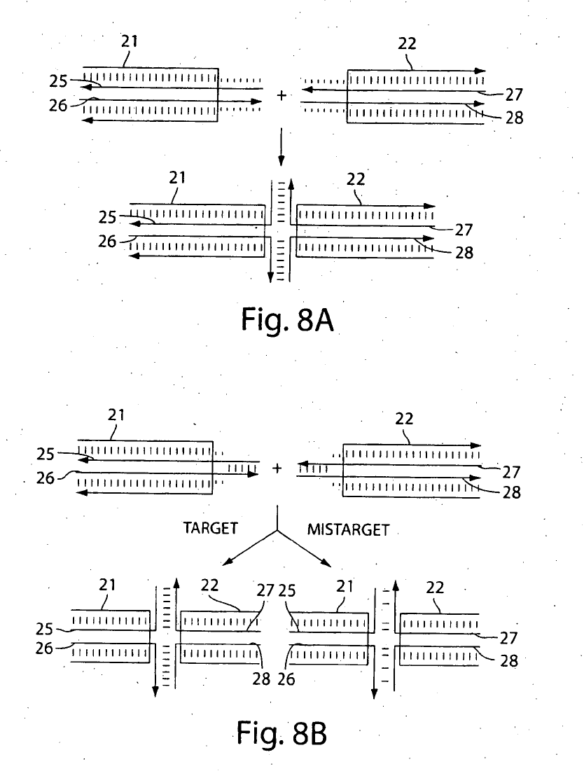

In some cases, even more stabilizers may be used to stabilize the association of the nucleic acid scaffold portions, for instance, four stabilizers as is shown in FIG. 8. As a non-limiting example, in this figure, a first nucleic acid portion 21 and a second nucleic acid portion 22 are associated by the use of four stabilizers 25, 26, 27, and 28. As shown in FIG. 8A, a first portion of stabilizer 25 is substantially complementary to a portion of first nucleic acid 21, while a second portion is substantially complementary to a portion of stabilizer 27. Similar complementarily patterns are used with respect to stabilizers 26, 27, and 28. When assembled, the portions of the stabilizers complementary to each other are able to associate. In some embodiments, there may also be complementarities between different stabilizers. As a non-limiting example, in FIG. 8B, stabilizer 25 contains a first portion substantially complementary to a portion of first nucleic acid 21, and a second portion that is complementary to both a portion of stabilizer 26 and a portion of stabilizer 27. The complementary portions may be in the same or different regions, depending on the embodiment. Similar complementarily patterns are used with respect to stabilizers 26, 27, and 28. When stabilizers 25 and 26 are associated with nucleic acid 21, stabilizers 25 and 26 associate both with their respective portions of nucleic acid 21 and to each other. However, when nucleic acid 22 and stabilizers 27 and 28 are introduced, the complementary regions between stabilizers 25 and 27, and between 26 and 28, may cause the association of nucleic acid 21 and 22 to occur.

If there is a mistargeting (e.g., if stabilizers 27 and 28 are not the intended complements of 25 and 26, respectively), some association may still occur, as is illustrated in Example 8B. However, this is not energetically favorable, as there is an energy cost associated with separating the complementary portions of stabilizers 25 and 26, which may not be adequately compensated by the association of stabilizer 25 with stabilizer 27, and stabilizer 26 with stabilizer 28. Accordingly, such a mistargeted reaction is not energetically or thermodynamically favorable.

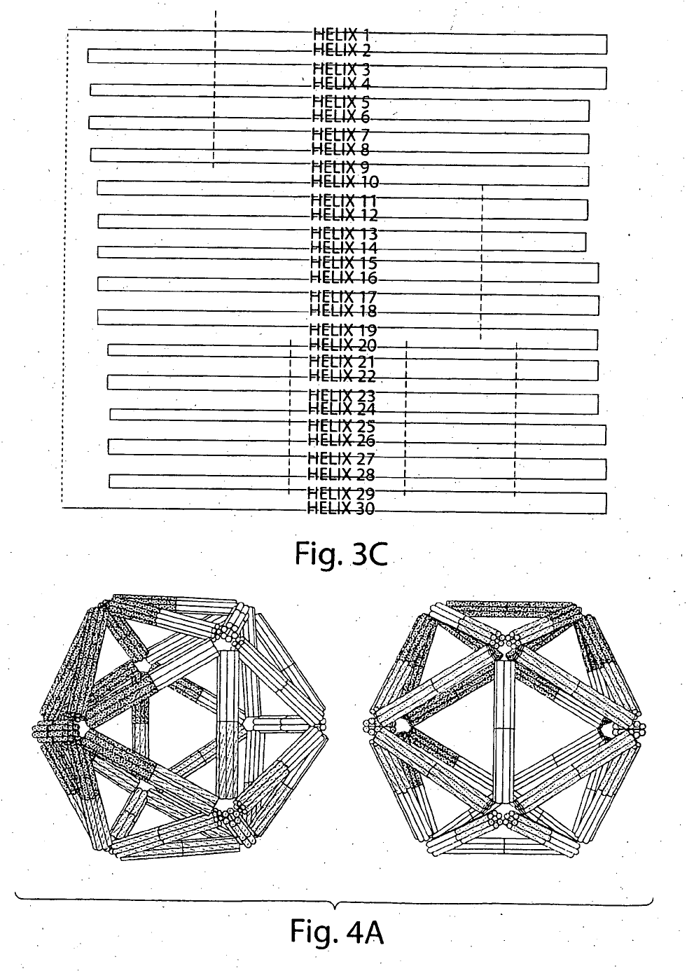

One non-limiting example of a method of forming a bundle or a nanotube of nucleic acid using nucleic acid stabilizers is as follows. Referring now to FIG. 2A, a layout 10 for a tetrahedral wireframe molecular structure is shown. In this figure, each edge of the tetrahedron (connecting two vertices together) is formed from a six-helix nanotube. The nanotubes themselves are composed of a nucleic acid scaffold 11 that wraps around layout 10, and shorter nucleic acid scaffolds 13 that loop only between two adjacent vertices. The nucleic acid strands may be stabilized to form a six-helix nanotube, e.g., as is shown in FIG. 1A, by using a plurality of stabilizers that connect adjacent nucleic acid strands in order to stabilize them. Any number of nucleic acids may be formed into a nucleic acid nanotube, for example, 4, 5, 6, 8, 10, 12, 14, 16, 18, 20, 24, 30, 42, 54, 66, 78, or 90 or more strands may be formed into such a nanotube. An example of a layout is shown in FIG. 3A (to form a tubular structure, the first and last strands are also connected, thereby causing the structure to curl to form a tube). In this figure, thick lines 21 indicate the nucleic acid strands (i.e., the nucleic acid scaffold forming the molecular structure), while thin lines 22 indicate the stabilizer strands connecting adjacent nucleic acid strands, thereby immobilizing the strands relative to each other, where the stabilizer strands comprise a first portion complementary to a first nucleic acid strand and a second portion complementary to a second, adjacent nucleic acid strand. Thus, by using stabilizers with suitable complementary sequences, any two arbitrary nucleic acid portions may be immobilized relative to each other, and bundles or struts such as the six-helix nanotubes shown, as well as larger structures, such as the tetrahedron shown in FIG. 2B, may be prepared. FIG. 3B shows a similar layout for a ten-helix nucleic acid nanotube, shown in FIG. 1B. To increase stability, instead of forming the ten-helix nucleic acid nanotube with nucleic acid strands that only run from one end to the other, here, some of the nucleic acid strands have been caused to loop around the nanotube. These “cross-over” locations within the bundle can be systematically or randomly chosen, depending on the application. FIG. 3C shows a similar layout for a 30-helix bundle nanotube.

Accordingly, the bundles may be used to form a three-dimensional molecular structure, i.e., a structure where all of the dimensions of the structure are greater than the approximate thickness of a nucleic acid strand, that is, greater than about 10 nm. Thus, the bundles may be thought of as “struts” which form such a molecular structure, such as a wireframe molecular structure. The entire three-dimensional molecular structure, in some embodiments, can be formed of bundles or nanotubes of nucleic acid. In some cases, each dimension of the three-dimensional molecular structure is independently greater than about 10 nm, for example, at least about 20 nm, at least about 30 nm, at least about 40 nm, at least about 50 nm, at least about 75 nm, at least about 100 nm, etc. The three-dimensional molecular structure may be substantially “globular” in some cases (i.e., where all three dimensions of the molecular structure are approximately the same), or one or more dimensions of the structure may be different (e.g., larger or smaller). In some cases, the three-dimensional molecular structure is non-planar, i.e., the structure does not have a shape in which one dimension is substantially smaller than the other two dimensions.

In one set of embodiments, the molecular structure is a wireframe structure, i.e., the molecular structure defines a geometrical shape comprising a plurality of vertices connected by edges or pathways. The wireframe molecular structure may be described as a geometric model that describes a three-dimensional shape by outlining its edges, where the edges may be formed from nucleic acids and are connected together at vertices or “corners” within the molecular structure. The edges or pathways may be straight, or curved in some cases. There may be any number of vertices and/or edges or pathways within the structure, for example, 3 or more vertices, 4 or more vertices, 5 or more vertices, 6 or more vertices, 8 or more vertices, 10 or more vertices, 12 or more vertices, 16 or more vertices, 20 or more vertices, etc. The edges or pathways can be defined as connecting two vertices together. The vertices are defined by locations where at least three distinct edges or pathways emanate, i.e., at least three distinct edges or pathways edges meet at a common point defining the vertex. In some cases, a vertex may include more than three edges or pathways, for example, the vertex may be defined by four or five edges or pathways that meet at a common point. Within a molecular structure, each vertex may independently have the same number, or different numbers of edges or pathways. For example, each vertex within a wireframe structure may have at least three pathways emanating therefrom (or only three pathways emanating therefrom), at least four pathways emanating therefrom (or only four pathways emanating therefrom), etc.

The wireframe structure may define a three-dimensional molecular structure. In certain embodiments, as discussed in detail below, the wireframe structure may define an interior space. In some embodiments, the molecular structure may be a “closed” structure, i.e., each vertex within the structure has at least three distinct edges or pathways edges emanating therefrom, and there are no edges or pathways within the molecular structure that end at a single point that is not a vertex. As discussed herein, the pathways or edges may be defined within the molecular structure by nucleic acids, and/or bundlers or nanotubes of nucleic acids, e.g., as described above. In some cases, each pathway within a molecular structure of the invention is defined by a bundle or a nanotube of nucleic acid.

In some cases, a plurality of vertices and edges or pathways may define “faces” on the wireframe molecular structure. For example, three vertices, connected by three edges, may define a triangular face; four vertices, connected by four edges, may define a rectangular face, a square face, or a rhombal face; five vertices, connected by five edges, may define a pentagonal face; etc. Accordingly, the wireframe molecular structure may define, for instance, a polyhedron having a shape defined by the plurality of vertices and edges or pathways.

For instance, a wireframe molecular structure, according to certain embodiments of the invention, may be a closed structure having one or more triangular faces. Triangular faces may be desirable in some cases, as a triangular structure is relatively rigid and cannot collapse merely due to its shape, unlike a square or other higher-order polygons. Accordingly, the molecular structure may be substantially rigid, as discussed below. However, in other embodiments of the invention, the wireframe structure may include faces that are not triangles, for instance, squares, pentagons, rhombii, octagons, etc. Thus, in some cases (for example, a cube formed from bundles or nanotubes of nucleic acids), no triangular faces are present. In other cases (for example, a truncated tetrahedron), both triangular and non-triangular faces may be present in the wireframe molecular structure.

For example, in one set of embodiments, the wireframe structure defines a polyhedron, i.e., a closed three-dimensional structure having a number of faces defined by edges and vertices. Typically, the faces in the polyhedron are polygonal, e.g., triangular, square, rectangular, rhombal, pentagonal, hexagonal, etc. One non-limiting example of polyhedra include the Platonic solids, where each face has the same shape and the lengths of each of the edges are all equal, i.e., the wireframe structure of the molecular structure may have a substantially tetrahedral shape (having 4 sides, 4 vertices, and 6 edges), a substantially cubic shape (having 6 sides, 8 vertices, and 12 edges), a substantially octahedral shape (having 8 sides, 6 vertices, and 12 edges), a substantially dodecahedral shape (having 12 sides, 20 vertices, and 30 edges), or a substantially icosahedral shape (having 20 sides, 12 vertices, and 30 edges).

However, the invention is not limited to the Platonic solids. For example, the wireframe structure of the molecular structure may be an Archimedean solid, i.e., a truncated tetrahedron, a cuboctahedron, a truncated cube, a truncated octahedron, a rhombicuboctahedron, a truncated cuboctahedron, a snub cube, an icosidodecahedron, a truncated dodecahedron, a truncated icosahedron, a rhombicosidodecahedron, a truncated icosidodecahedron, or a snub dodecahedron; or the structure of the molecular structure may be a Catalan solid, i.e., a triakis tetrahedron, a rhombic dodecahedron, a triakis octahedron, a tetrakis hexahedron, a deltoidal icositetrahedron, a disdyakis dodecahedron, a pentagonal icositetrahedron, a rhombic triacontahedron, a triakis icosahedron, a pentakis dodecahedron, a deltoidal hexecontahedron, a disdyakis triacontahedron, or a pentagonal hexecontahedron; or a Johnson solid. Other non-limiting examples include a pyramid (having a polygonal base and an apex), a noncubic prism (having a polygonal base and a nontranslated copy of the base as the top), a nonoctahedral antiprism (having a polygonal base and a rotated copy of the base at the top), a bipyramid, (having a polygonal base and two apexes on either side of the base), a noncubic trapezohedra, or a cupola (formed by joining two polygons, one with twice as many edges as the other, by an alternating band of triangles and rectangles).

In some cases, the faces in the polyhedron are all triangular, i.e., each face of the molecular structure is defined by only three vertices and three edges or pathways. Thus, the molecular structure has the shape of a deltahedron. Examples of deltahedra include tetrahedrons, octahedrons, icosahedrons, snub disphenoids, triaugmented triangular prisms, gyroelongated square dipyramids, triangular dipyramids, pentagonal dipyramids, or bipyramids. Thus, in some cases, each vertex of the molecular structure is connected to at least two other vertices that are connected to each other.

The edges or pathways of the wireframe molecular structure may have any suitable length. For example, some or all of the edges or pathways may have a length of at least about 40 nm, at least about 50 nm, at least about 60 nm, at least about 75 nm, at least about 100 nm, or more. In some cases, the length may be greater than the persistence length of single or double stranded DNA, e.g., a length of at least about 50 nm. In some embodiments, as discussed below, the molecular structure remains substantially rigid even with edges or pathways larger than the persistence length of double stranded DNA. In certain cases, such lengths allow for relatively large molecular structures to be created. For example, the molecular structure may have a smallest dimension that is at least about 50 nm, at least about 75 nm, at least about 100 nm, at least about 150 nm, at least about 200 nm, at least about 500 nm, or more in some cases and, as mentioned, in some embodiments, the molecular structure may define an interior space that has similar dimensions.

As a specific example, a molecular structure such as a wireframe molecular structure having the shape of a Platonic, Archimedean, Catalan, or Johnson solid may also define an interior space. In some cases, the interior space is relatively large, for example, having a smallest dimension, internal of the molecular structure, of at least about 50 nm, at least about 75 nm, at least about 100 nm, at least about 150 nm, at least about 200 nm, at least about 500 nm, or more in some cases. In certain cases, as further discussed below, the interior space may be used to contain another molecule, such as a biological molecule (e.g., a protein, an enzyme, a drug, etc.), or other molecule, or even other molecular structures such as other molecular structures described herein (e.g., which may form a “nesting” of such molecular structures).

In some embodiments, the molecular structure is substantially rigid or non-collapsible, i.e., the molecular structure is able to retain its three-dimensional structure under various conditions. Typically, a molecular structure can be considered to be rigid if it is able to maintain its three-dimensional configuration in solution under ambient conditions. In some cases, the structure is rigid if it is formed of edges or pathways having a length greater than the persistence of the components that form the structure, but remains able to maintain its shape in solution. The persistence is, generally speaking, the average length one must travel along an object (e.g., along DNA) before a substantial change in direction is found. For instance, a six-helix DNA bundle is rigid if it is of a length greater than the persistence of the DNA forming the bundle (the persistence length of double stranded DNA is about 50 nm), but remains able to maintain its shape in solution (i.e., a straight bundle of DNA remains substantially straight in solution, and does not curve or fold, a bundle of DNA having the shape of a triangle does not denature or warp, etc.). As another example, a substantially rigid molecular structure having the shape of an icosahedron or a tetrahedron would not dissociate into free nucleic acids in solution, but would maintain its respective icosahedral or a tetrahedral shape. In one set of embodiments, the persistence of the edges or pathways may be at least about 100 nm, at least about 300 nm, at least about 500 nm, at least about 750 nm, or at least about 1000 nm. Rigidity may be created within the molecular structure, for example, due to the use of nucleic acid bundles or nanotubes, through the use of triangular arrangements of such nucleic acid bundles or nanotubes (e.g., triangular faces), or the like.

In certain embodiments, the molecular structure is anisotropic or nonsymmetric on a molecular level, even though the molecular structure defines a three-dimensional structure that is symmetric. As an example, a molecular structure may have a substantially icosahedral shape, where the molecular structure comprises one or more nucleic acid scaffolds, stabilized by one or more nucleic acid stabilizers able to associate with two or more nucleic acid portions. The nucleic acid scaffolds may be substantially free of self-complementary regions or repeat units, such that, on the molecular level, substantially each edge or vertex of the icosahedra molecular structure is defined by unique sequences of nucleic acid bases. As discussed below, these unique sequences can each be individually targeted, e.g., using complementary nucleic acid sequences, such that molecules can be anisotropically immobilized relative to the molecular structure. Accordingly, for example, a molecule may be immobilized relative to a first vertex or edge within the molecular structure without being immobilized relative to other vertices or edges within the molecular structure. As a specific non-limiting example, a receptor for a ligand can be attached to one portion of the molecular structure.

In some cases, a lipid may be associated with a composition of the invention. For example, in one embodiment, a liposome may contain a composition of the invention, e.g., producing a liposome rigidified by a nucleic acid. In another embodiment, a lipid membrane may be associated with at least a portion of a composition of the invention, for example, by being associated with one or more edges or pathways of the molecular structure, by forming a “sheet” across one or more faces of a molecular structure, etc. In some cases, the lipid may be immobilized relative to the molecular structure using one or more surfactants, which may have a positively charged portion (e.g., attracted to the nucleic acid, which is typically negatively charged) and a lipophilic portion that can associate with the lipids. Such structures may be created, for example, by exposing a wireframe molecular structure comprising nucleic acid to a lipid and/or a surfactant in solution.

In certain embodiments, the molecular structure may contain one or more molecules in an interior space within the molecular structure, for example, a biological molecule (e.g., a protein, an enzyme, a drug, etc.). The contained molecule may be immobilized relative to a portion of the molecular structure (e.g., to a vertex or an edge or pathway), or “freely” contained within the interior space, e.g., such that it is not covalently bound to a portion of the molecular structure. Uses of such molecular structures are discussed below. The molecule may be trapped within the molecular structure, for example, by forming the molecular structure in the presence of the molecule to be trapped inside. For instance, as discussed below, the final molecular structure may be prepared by combining a plurality of nucleic acids together in solution, serially or simultaneously; during this process, the molecule to be trapped inside may also be present in solution. A molecule immobilized to a portion of a molecular structure may have a sequence that is substantially complementary and/or specifically binds to a specific or unique sequence of the final molecular structure; as mentioned, in some embodiments, the final molecular structure is anisotropic and a specific edge or vertex can be individually targeted.

According to one embodiment of the invention, a layout of a molecular structure of the invention may be prepared as follows. A desired molecular structure, and one or more routes that proceed through each vertex and each edge of the molecular structure is identified. In some embodiments, the routes that wrap through the molecular structure may overlap (e.g., if each vertex within the molecular structure includes an odd number of edges). Depending on the wireframe molecular structure desired, in some embodiments, the shape may be formed by first drawing out a polyhedral net of the wireframe structure, and identifying which edges need to be joined (e.g., using nucleic acid stabilizers) to produce the final molecular structure. If some or all of the edges or pathways comprise bundles or nanotubes of nucleic acids, such structures may be designed by including, in this route, multiple connections between each vertex. As a non-limiting example, a six-helix nucleic acid bundle may be designed between two vertices by having a route go back and forth between the two vertices, as is shown in FIG. 2A with route 13, and/or a route may go around the molecular structure multiple times, including the same pathway more than once, as is shown with path 11. In some cases, one or more edges of the molecular structure may be divided into partial routes, such as is shown with edge 16 in FIG. 2A. Virtually any routing can be used to layout the molecular structure, including any combination of the above. For example, any combination of routes may be used to create a six-helix bundle between any two vertices in a molecular structure.

The routes may then be mapped to one or more nucleic acids (i.e., one or more nucleic acid scaffolds), for example, a relatively long nucleic acid having a length of at least about 500 nucleotides, at least about 1,000 nucleotides, at least about 3,000 nucleotides, at least about 10,000 nucleotides, at least about 30,000 nucleotides, etc., depending on the size of the final molecular structure desired. For longer edges or pathways, more nucleotides may be included between each vertex. If the nucleic acid scaffold is not sufficiently long, multiple nucleic acid scaffolds can be used within the molecular structure.

By examining the routes as discussed above, portions of the final molecular structure that should be immobilized relative to each other can be identified. Such portions include bundles or nanotubes forming the edges of the molecular structure, as well as half-pathways that are joined to form a complete pathway connecting two vertices in the final molecular structure. If the nucleic acid scaffolds used to form the final molecular structure are substantially free of self-complementary regions or repeat units, then such nucleic acid stabilizers will have a unique sequence that is determined by the two nucleic acid portions to be immobilized relative to each other, i.e., the nucleic acid stabilizer will have a first portion complementary to a first nucleic acid strand and a second portion complementary to a second nucleic acid strand. Accordingly, each of the nucleic acid stabilizes will associate with the nucleic acid scaffolds in specific locations, which then allow the final molecular structure to be formed. Such nucleic acid stabilizers can be synthesized using techniques known to those of ordinary skill in the art, for example, using Caruthers synthesis.

The final molecular structure may then be prepared by combining these nucleic acids together. The nucleic acids may be combined together, e.g., all at once or serially. For instance, in some embodiments, the final molecular structure may be prepared by starting with a one or more nucleic acid scaffolds, adding one (or a relatively small number) of nucleic acid stabilizers, and allowing the nucleic acids to interact. In some cases, interaction between complementary regions may cause the final molecular structure to spontaneously self-assemble. In other cases, however, some heat may be required, for instance, a temperature of about 50° C., about 60° C., about 70° C., or about 80° C. may be used to promote assembly.

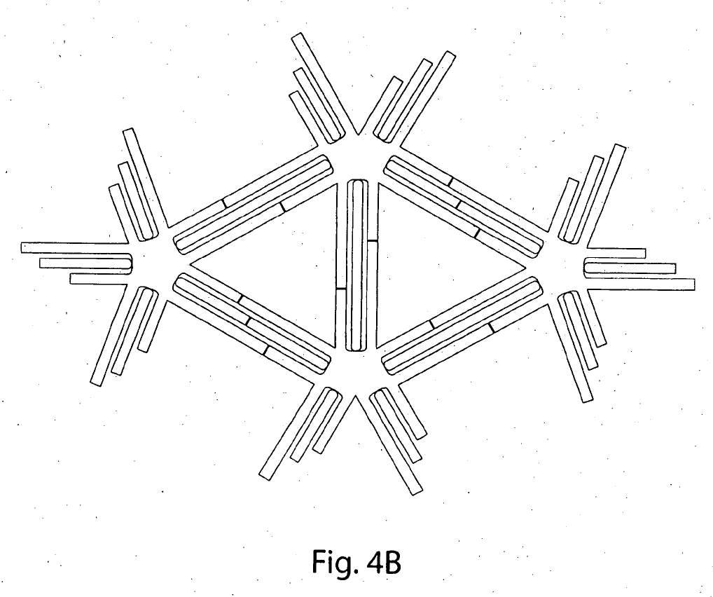

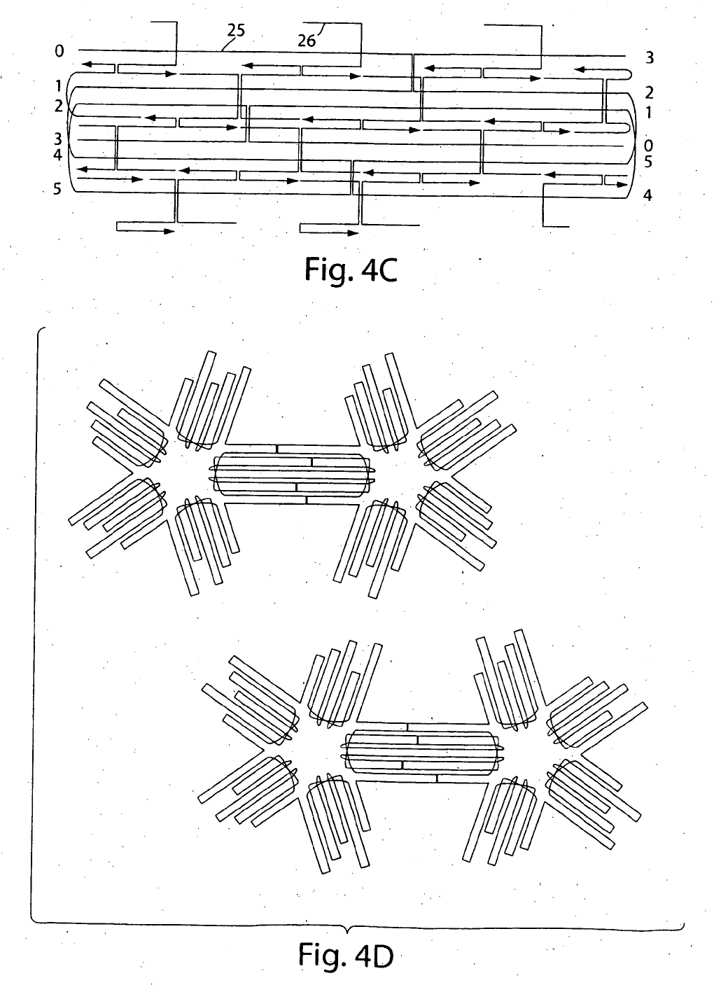

As a specific non-limiting example, a layout for an icosanderal molecular wireframe structure is now described. In an icosahdron, each vertex includes 5 edges that emanate therefrom, and the overall structure has 20 faces. In this example, multiple wireframe structures are created, which are then combined to form the overall icosahedral structure, as shown in FIG. 4A. Here, three “double-triangle” structures, as shown in FIG. 4B, are assembled together; each edge of the wire frame structure is a six-helix nucleic acid nanotube. The nanotubes for each edge are shown in FIG. 4C. In this figure, thick lines 25 indicate the nucleic acid strands (i.e., the nucleic acid scaffold forming the molecular structure), while thin lines 26 indicate the stabilizer strands connecting adjacent nucleic acid strands, thereby immobilizing the strands relative to each other, where the stabilizer strands comprise a first portion complementary to a first nucleic acid strand and a second portion complementary to a second, adjacent nucleic acid strand. In the final icosahedral molecular structure, an initial nucleic acid is used to form all three double triangle structures; to ensure uniqueness of the three structures, different cyclic permutations of the initial nucleic acid are used. Thus, each double triangle is chemically unique, and the final icosahedral structure is a heterotrimer of the three double triangles.

FIG. 4D illustrates another embodiment, where an icosahedral structure in which each edge of the structure is a ten-helix nucleic acid bundle is shown. FIG. 4D shows two layouts used to form the molecular structures used to form the final structure; in this example, five of these structures were used to assemble the final icosahedral structure. As can be seen, the layout includes a number of pathways extending two vertices, as well as a number of half-pathways that are joined together in the final structure.

Another aspect of the invention is directed to uses of such molecular structures. For instance, as previously described, the molecular structure may be one that defines an interior space that can be used to contain one or more molecules, such as a biological molecule (e.g., a protein, an enzyme, a drug, etc.). The molecule contained within the molecular structure is thus at least partially isolated from the environment surrounding the molecular structure, and in some cases, such isolation may be enhanced using lipids associated with the molecular structure. For example, a liposome containing a wireframe molecular structure may be formed, or one or more lipid membranes may be associated with the faces of the molecular structure.

In one set of embodiments, a molecule within the interior space of the molecular structure may be a drug molecule. By containing the drug molecule within the molecular structure, the drug molecule can be isolated from the external environment. Accordingly, such a molecular structure may be used as a carrier to deliver the drug to a target, e.g., in a subject, without exposure of the drug to other targets or locations in the subject. In some embodiments, the molecular structure may be targeted using one or more receptors or ligands attached to the exterior of the molecular structure, which may be used to target the molecular structure to a particular location. In one embodiment, the receptor or ligand may be asymmetrically positioned with respect to the molecular structure (for example, at a particular vertex), for instance, if the molecular structure is asymmetric. In some cases, a relatively large number of ligands may be attached to the molecular structure per unit length, as there may be a greater surface area in the molecular structure, e.g., due to the presence of bundles of nucleic acids.

In another embodiment, however, one or more of the nucleic acids forming the molecular structure may itself be an active agent able to affect a subject. For example, the nucleic acid may encode a protein or an enzyme, e.g., for gene therapy, or the nucleic acid may encode a sequence for gene silencing purposes. Thus, in one embodiment, a molecular structure comprising nucleic acids may be delivered to a subject. In some cases, the molecular structure may contain a drug; but in other cases, no drug may be present. In some cases, the molecular structure within the subject may be at least partially disassembled (e.g., within a target cell), thereby releasing the nucleic acids, which may be then be expressed by the cell or otherwise act on the cell.

In another set of embodiments, a molecule contained within the interior space of the molecular structure may be further studied, e.g., using NMR analysis or electron microscopy. Techniques such as NMR may benefit, e.g., with improved resolution, when the molecule to be studied is trapped within a contained environment, such as within the interior space.

In yet another set of embodiments, the interior space of the molecular structure may be altered, often in a systematic way, by immobilizing of one or more molecules to the interior space, e.g., to an edge or a vertex of the molecular structure. For example, an acid environment may be created within the interior space by immobilizing one or more acids to the interior.

The present invention also provides any of the above-mentioned compositions in kits, optionally containing instructions, in another aspect. “Instructions” can define a component of promotion, and typically involve written instructions on or associated with packaging of compositions of the invention. Instructions also can include any oral or electronic instructions provided in any manner. The “kit” typically defines a package including any one or a combination of the compositions of the invention and the instructions, but can also include the composition of the invention and instructions of any form that are provided in connection with the composition in a manner such that one of ordinary skill in the art would clearly recognize that the instructions are to be associated with the specific composition.

The kits described herein may also contain one or more containers, which may contain the inventive composition and other ingredients as previously described. The kits also may contain instructions for mixing, diluting, or otherwise processing the compositions of the invention in some cases. The kits also can include other containers with one or more solvents, surfactants, preservative and/or diluents (e.g., normal saline (0.9% NaCl), or 5% dextrose) as well as containers for mixing, diluting, or otherwise processing the compositions.

The compositions of the kit may be provided as any suitable form, for example, as liquid solutions or as dried powders. When the composition provided is a dry powder, the composition may be reconstituted by the addition of a suitable solvent, which may also be provided. In embodiments where liquid forms of the composition are used, the liquid form may be concentrated or ready to use. The solvent will depend on the compound and the mode of use.

As used herein, “promoted” includes all methods of doing business including, but not limited to, methods of selling, advertising, assigning, licensing, contracting, instructing, educating, researching, importing, exporting, negotiating, financing, loaning, trading, vending, reselling, distributing, repairing, replacing, insuring, suing, patenting, or the like that are associated with the methods or compositions of the invention as discussed herein. Methods of promotion can be performed by any party including, but not limited to, personal parties, businesses (public or private), partnerships, corporations, trusts, contractual or sub-contractual agencies, educational institutions such as colleges and universities, research institutions, governmental agencies, etc. Promotional activities may include communications of any form (e.g., written, oral, and/or electronic communications, such as, but not limited to, e-mail, telephonic, Internet, Web-based, etc.) that are clearly associated with the invention.

In one set of embodiments, the method of promotion may involve one or more instructions. As used herein, “instructions” can define a component of instructional utility (e.g., directions, guides, warnings, labels, notes, FAQs or “frequently asked questions,” etc.), and typically involve written instructions on or associated with the invention and/or with the packaging of the invention. Instructions can also include instructional communications in any form (e.g., oral, electronic, audible, digital, optical, visual, etc.), provided in any manner such that a user will clearly recognize that the instructions are to be associated with the invention, e.g., as discussed herein.

U.S. Provisional Patent Application Ser. No. 60/923,831, filed Apr. 17, 2007, entitled “Wireframe Nanostructures,” by Shih, is incorporated herein by reference in its entirety.

The following examples are intended to illustrate certain embodiments of the present invention, but do not exemplify the full scope of the invention.

### Example 1

This example illustrates the design and characterization of scaffolded DNA origami wireframe tetrahedra with six-helix bundle struts. A mechanically robust implementation of wireframe polyhedra is to build with DNA-nanotubes as struts. Such a design was pursued within the theme of a scaffold that is folded by staple oligonucleotides (Rothemund, P. W. K., “Folding DNA to Create Nanoscale Shapes and Patterns,” Nature, 440:297-302 (2006)) into a two-dimensional branched tree, where the terminal branches join to form additional struts.

In the current example, a tetrahedron was designed and assembled, as shown in FIG. 2B. The layout is shown in FIG. 2A. The length between dotted lines in this figure is 42 basepairs. Large two-sided arrows 18 indicate terminal branches that are complementary for the formation of a strut. The nucleic acid scaffold was based on the nucleic acid of M13 bacteriophage. Each strut had 126 basepairs per double helix, where there are six double helices per strut (FIG. 3A). Dark lines indicate the scaffold, while light lines indicate “staple” stabilization strands. Each staple strand was 42 bases long, and had three segments complementary to a 14-base segment of the scaffold. Of the six helices per strut, only two participated in interstrut connections at the vertices. These “core” helices, which have the closest proximity to the center of the tetrahedron, were adjacent within each strut. As there are six struts in a tetrahedron, the structure contained 126×6×6=4,536 basepairs. There were twelve unpaired bases between non-core helices at the vertices, and three unpaired bases between core helices across struts at the vertices. Thus a total of 4,536+12×6×4+3×4×4=4,872 bases of the scaffold were used for this object (it should be noted that the number of bases can be arbitrarily chosen). This object was folded with the M13-derived 7,308-base scaffold described above.

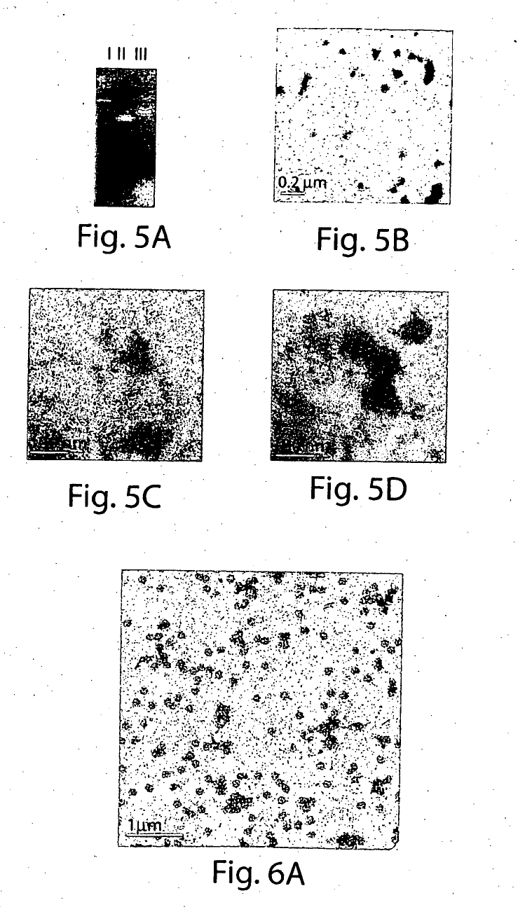

The folded tetrahedra were analyzed by native agarose gel electrophoresis and negative-stain electron microscopy (FIG. 5). FIG. 5A shows native agarose-gel electrophoresis: I, 1 kb ladder, II, naked scaffold, III, folded tetrahedra. FIG. 5B shows negative-stain electron micrograph. As expected, the struts based on DNA nanotubes appeared more rigid than simple double helices (which have a persistence length of about 50 nm). The length of the struts was consistent with the 43 nm expected. The imaged objects were consistent with structures that are folding into the target shape. Folding is not perfect, however, as some particles can be found as dimers and other multimers. The scale bars in FIGS. 5C and 5D are each 100 nm. In FIG. 5D, a tetrahedral dimer can be seen. Thus, in some embodiments of the invention, multimerization of the scaffolded structures can be observed.

### Example 2

This example shows the design and characterization of a scaffolded DNA origami wireframe icosahedron with six-helix bundle struts. An icosahedron encloses a space that is more than eighteen times as large than a tetrahedron with the same length struts. A wireframe icosahedron design was pursued in this example, also using a scaffolded DNA approach. As with the tetrahedron, every strut in this example was composed of six double helices that were 126 base pairs long each. Unlike the tetrahedron, the two helices of each strut that connected to neighboring struts within each vertex lay on opposite faces of the six-helix bundle DNA nanotube, e.g. helices i and i+3 (FIG. 4C). Most staple strands were 42 bases long, and were formed from three segments complementary to a 14-base segment of the scaffold. This altered arrangement accommodated the less acute angle of the struts at the vertex. There are 30 struts in an icosahedron, thus there were 30×6×126=22,680 base pairs in this icosahedron. This is larger than a single nucleic acid scaffold derived from M13; thus the wireframe icosahedron was built on three separate scaffolds, each folding into a double triangle with four vertices (FIG. 4D). Unpaired bases connect the helices at the vertices, for a total of 1,620 bases for the entire icosahedron. Thus each scaffold was 8,100 bases long. The three double triangles have the same structure and use the same scaffold, although three different cyclic permutations of that scaffold were threaded through the structure for the three structures. Thus each double triangle is chemically distinct, and the final icosahedron was a heterotrimer of double triangles.

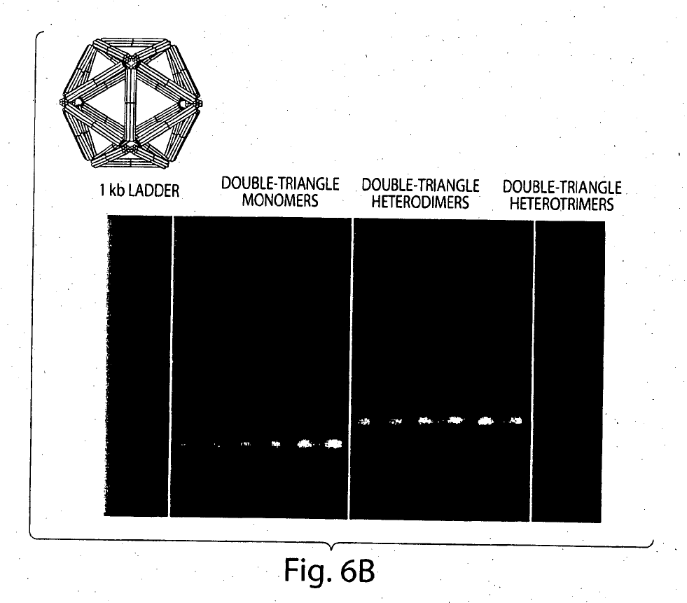

The folded icosahedra were analyzed by negative-stain electron microscopy (FIG. 6A) and native agarose gel electrophoresis (FIG. 6B). As with the tetrahedron in Example 2, the struts based on DNA nanotubes appeared more rigid than simple double helices (which have a persistence length of about 50 nm). The length of the struts was consistent with the 43 nm expected. The imaged objects were consistent with structures that are folding into the target shape. Folding is not perfect, however, as some particles can be found as aggregates larger than a heterotrimer.

### Example 3

This example illustrates the design of scaffolded DNA origami wireframe icosahedron with ten-helix bundle struts. Icosahedra built with ten-helix bundle struts (FIGS. 1B and 3B) may be more rigid than icosahedra built with six-helix bundle struts. In FIG. 3B, most of the staple strands are 42 bases long, and are formed of three segments complementary to a 14-base segment of the scaffold. Each strut is composed of double helices that are 126 base-pairs long.

### Example 4

This example illustrates various methods useful in certain embodiments of the invention.

Gel electrophoresis was performed as follows. Native agarose gel electrophoresis is performed with 45 mM Tris-borate, 1 mM EDTA, 11 mM MgCl₂ added (10 mM Mg²⁺ final), pH 8.3, 5 V/cm.

Recombinant M13 bacteriophage plasmid (p8100) was prepared by replacement of the BamHI-XbaI segment of M13mp18 by a polymerase chain reaction-generated 851 base pair (bp) fragment encoding a sequence amplified from the lambda phage genome.

Production of M13 bacteriophage single-stranded DNA was performed as follows. Recombinant M13 bacteriophage RF dsDNA was transformed into JM109 cells and grown overnight at 37° C. on an LB-agar plate. A single, well-isolated plaque was used to inoculate 2 mL of 2xYT medium in a 14 mL sterile culture tube and agitated for 8 hours at 37° C. Bacterial cells were pelleted by centrifugation and phage was recovered from the supernatant by polyethylene glycol fractionation (incubation on ice for 30 minutes using a final concentration of 4% PEG8000, 0.5 M NaCl) followed by centrifugation. The phage was resuspended in 100 microliters of 10 mM Tris·Cl pH 8.5 and labeled “pre-inoculation phage.” E. coli JM109 cells were grown overnight in 3 mL of 2xYT medium at 37° C. The 3 mL of JM109 culture was added to a 2 L flask containing 300 mL 2xYT medium supplemented with MgCl₂ to 5 mM final concentration and incubated at 37° C. on a shaker at 300 rpm. When the bacterial culture reached A₆₀₀=0.5, 50 microliters of the “pre-inoculation phage” stock was added. The infected culture was grown at 37° C., shaking at 300 rpm for an additional 4 hours. The phage was recovered as described above, and resuspended in 3 mL 10 mM Tris·Cl pH 8.5 and labelled “inoculation phage.” A titer of “inoculation phage” was measured by plating out serial dilutions using saturated JM109 culture and LB-top agar plates. A titer of JM109 cells at A₆₀₀=0.5 was measured by plating out serial dilutions on LB-agar plates. For nanomole-scale production of phage, twelve 2 L flasks each containing 300 mL 2xYT medium supplemented with 5 mM MgCl₂, were inoculated with 3 mL overnight JM109 culture and incubated at 37° C. shaking at 300 rpm. When density reached A₆₀₀=0.5, each flask was infected with “inoculation phage” at an MOI=1. The phage was harvested as described, and resuspended in 0.5% of the original culture volume in 10 mM Tris·Cl pH 8.5.

Single-stranded DNA was isolated from phage by alkaline/detergent denaturation as follows. Two volumes of lysis buffer (0.2 M NaOH, 1% SDS) were added to the resuspended phage, followed by 1.5 volumes neutralization buffer (3 M KOAc pH 5.5). Lysed phage was centrifuged for 10 minutes at 16000 rcf. The supernatant was combined with one volume of 200 proof ethanol and centrifuged for 10 minutes at 16000 rcf. Pelleted ssDNA was washed twice with 75% ethanol, centrifuged, and resuspended in 5% of the original culture volume in 10 mM Tris·Cl pH 8.5. The concentration of the recovered ssDNA was estimated on a UV/visible spectrophotometer (Beckman Coulter) using an extinction coefficient=37.5 micrograms/mL for A₂₆₀=1.

Folding of DNA nanostructures was conducted as follows. Desalted DNA oligonucleotides, normalized by concentration to 50 micromolar, were purchased from Invitrogen. Oligonucleotides were pooled to create stocks at 250 nM each strand. The folding mixture contained 50 mM HEPES pH 7.5, 50 mM NaCl, and 30 mM MgCl₂, 10 nM scaffold, 100 nM each staple strand. The mixture was processed on a thermal cycler (MJ Research Tetrad) with the following program:
1. 80.0° C. for 5:00
2. 80.0° C. for 2:00 (−1° C. per cycle)
3. Goto 2, 60 times
4. End

For the icosahedron, double triangles were heated to 60° C. for 2 min, then mixed at 60° C. and incubated at 60° C. for two hours.

Experimental protocols for negative-staining of samples and electron microscopic imaging were as follows. After folding, 3 microliters of folded sample were deposited on a glow-discharged carbon-coated copper grid and incubated at room temperature for 20 seconds. The liquid was wicked away with Whatman filter paper, then the sample was quickly dipped in a suspension of filtered 0.7% uranyl formate. The liquid was wicked away on Whatman filter paper, and the sample is incubated in a drop of uranyl formate for 20 seconds. The excess liquid was wicked away, and the grid dried with vacuum aspiration. Electron microscopy was performed on a Philips CM10 with 100 kV tungsten filament providing illumination.

### Example 5

This example illustrates sequences that were prepared using an embodiment of the invention. In this example, a scaffold nucleic acid sequence was folded to produce a regular icosahedral shape in which each edge was defined by a six-helix nucleic acid bundle.

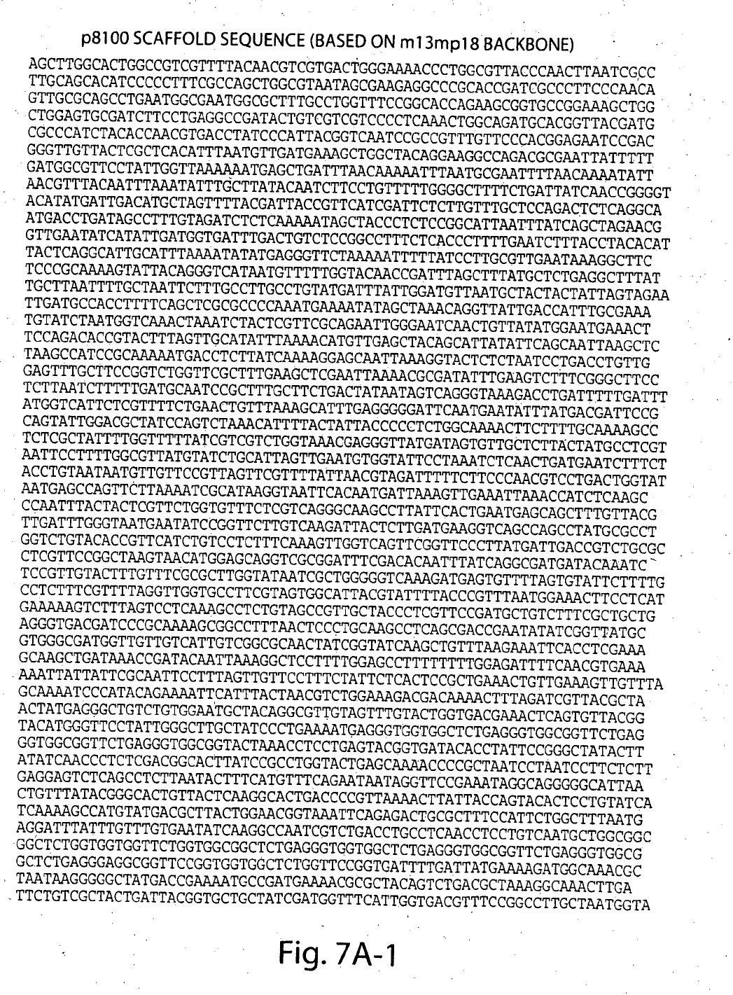

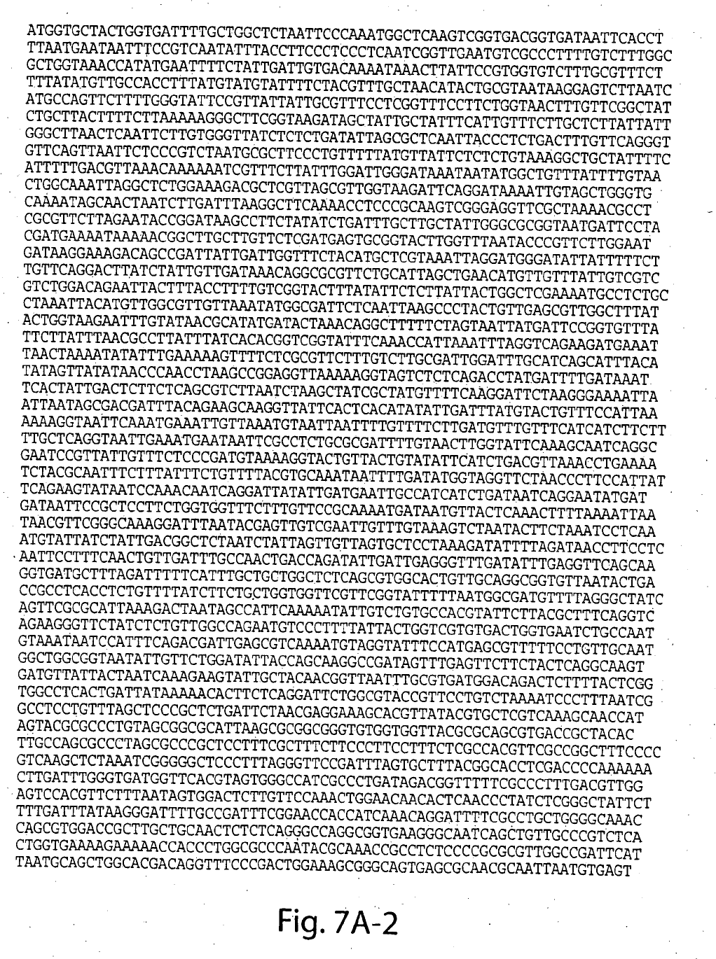

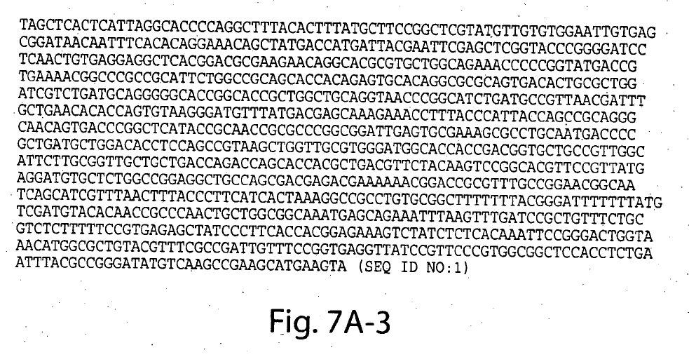

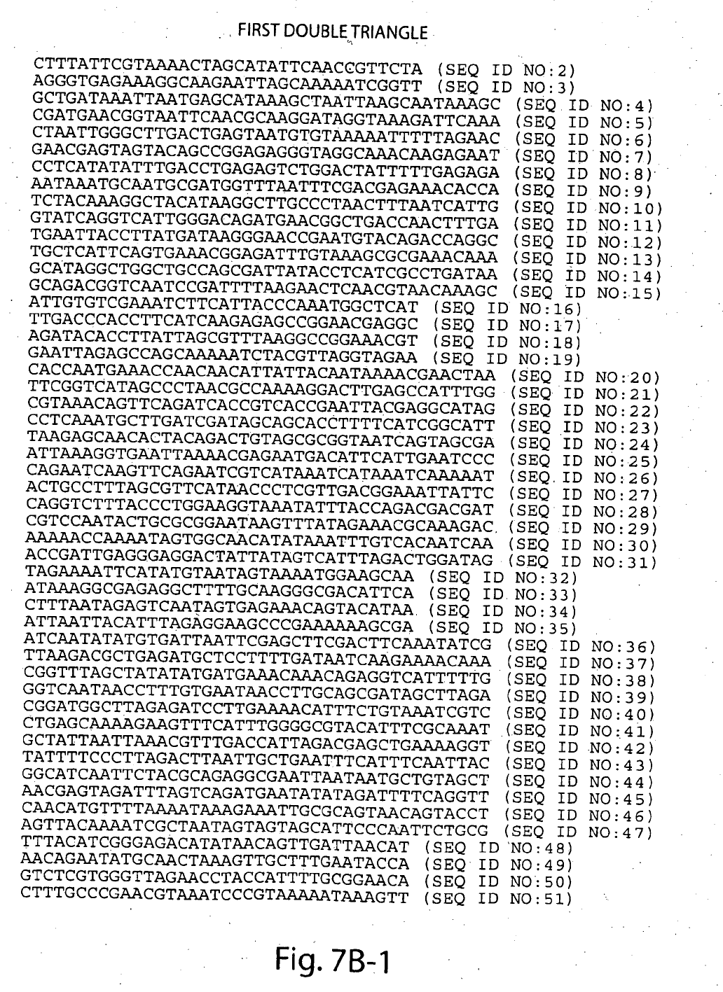

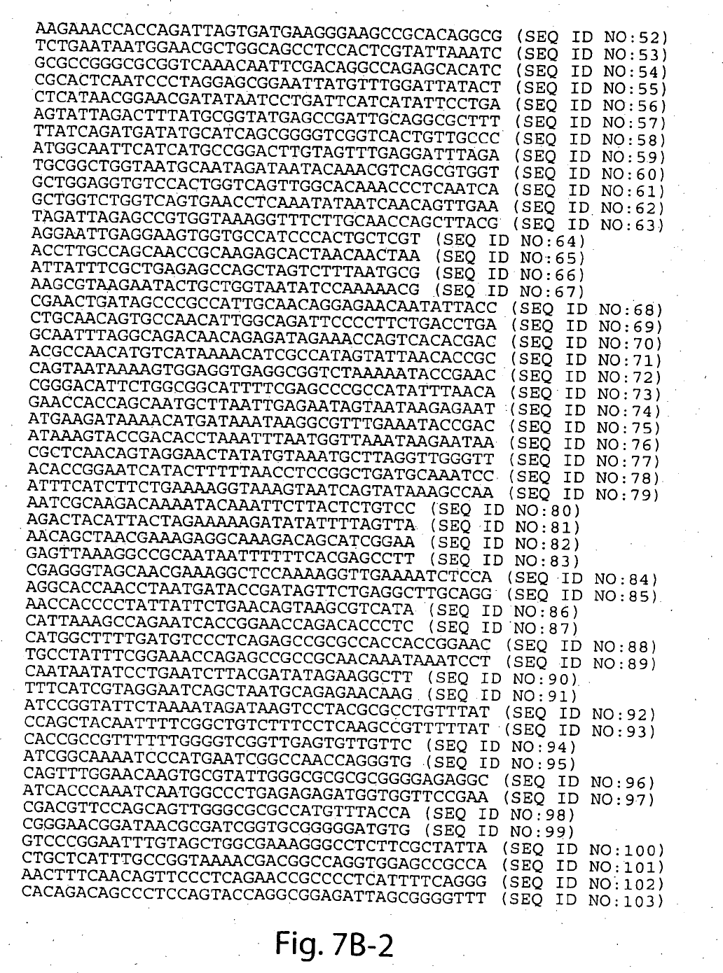

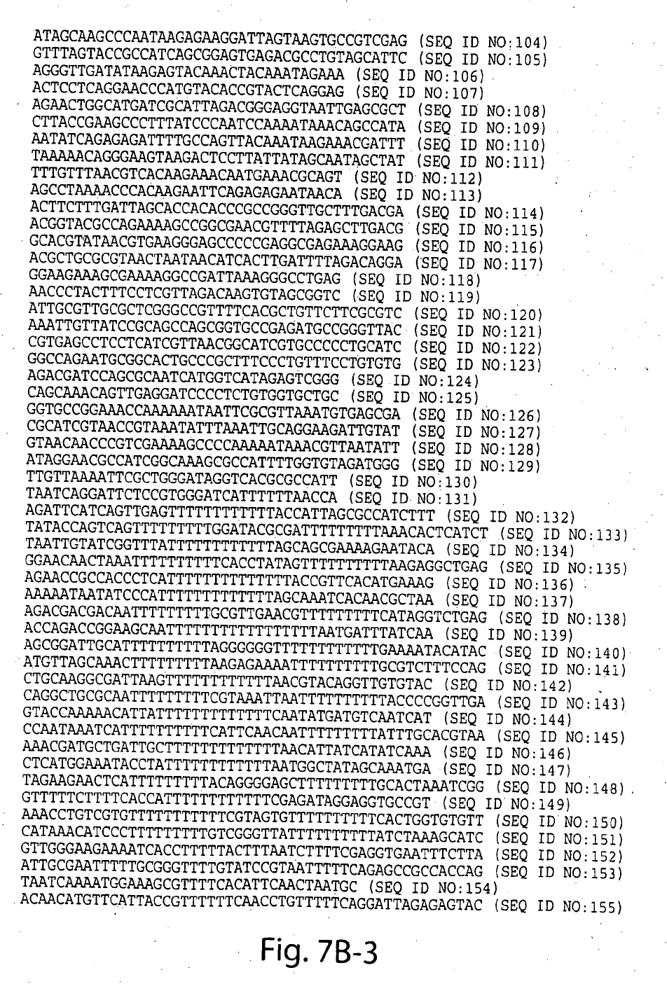

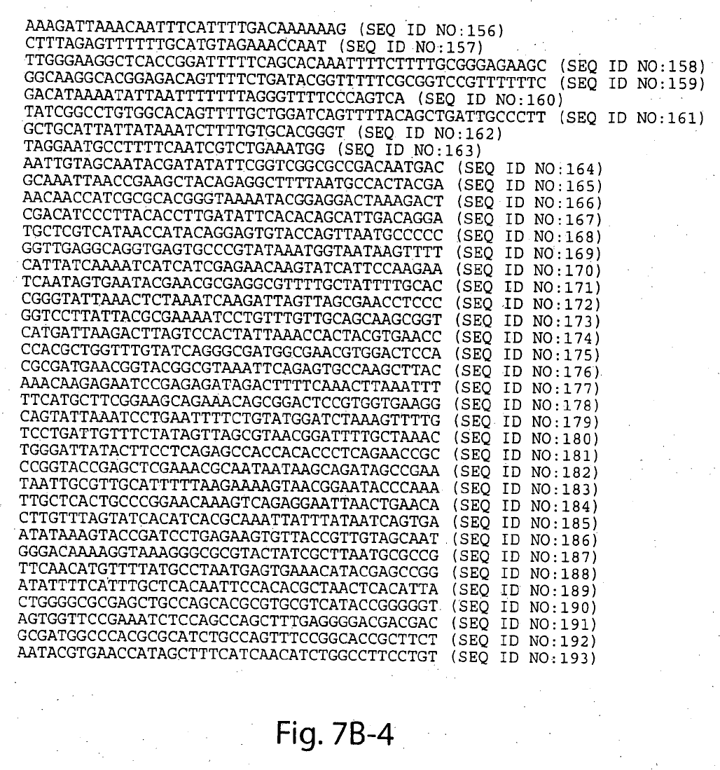

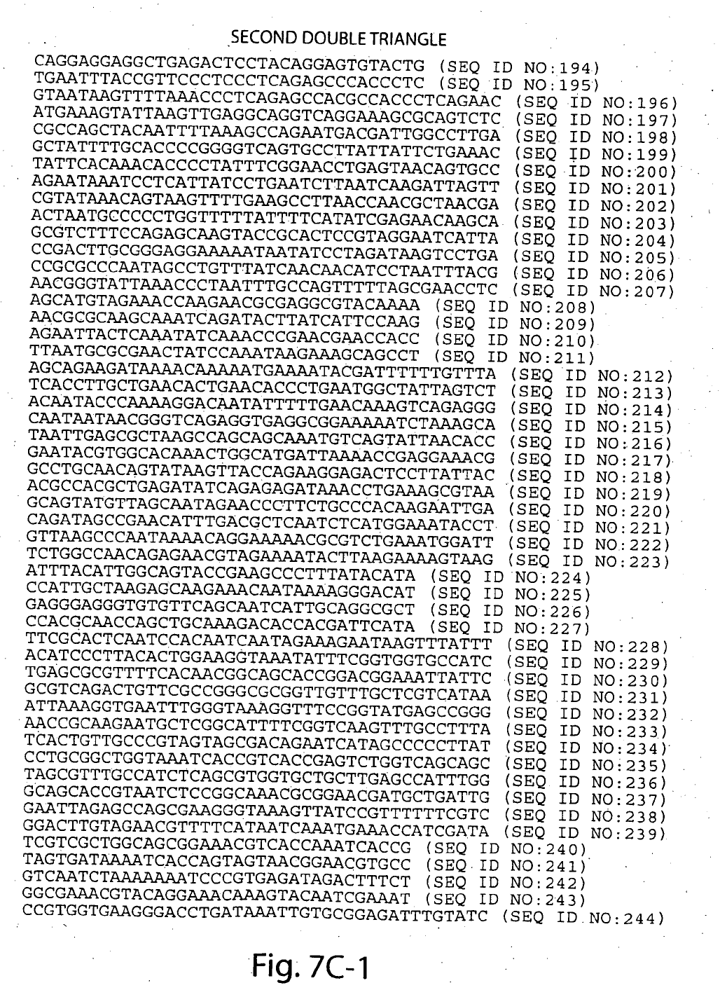

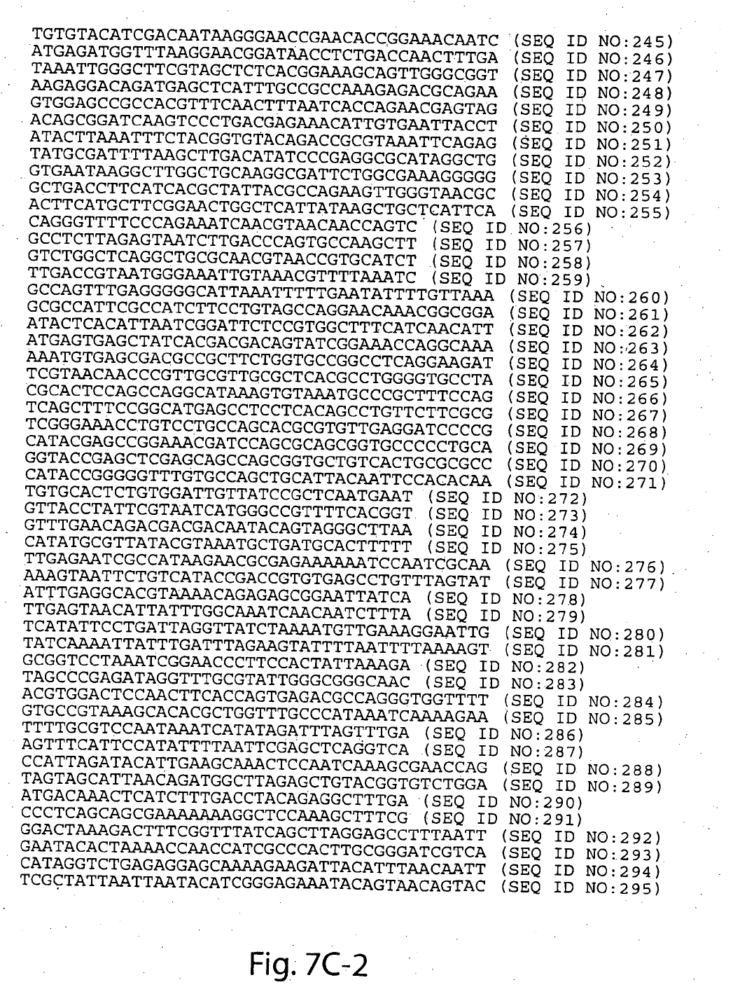

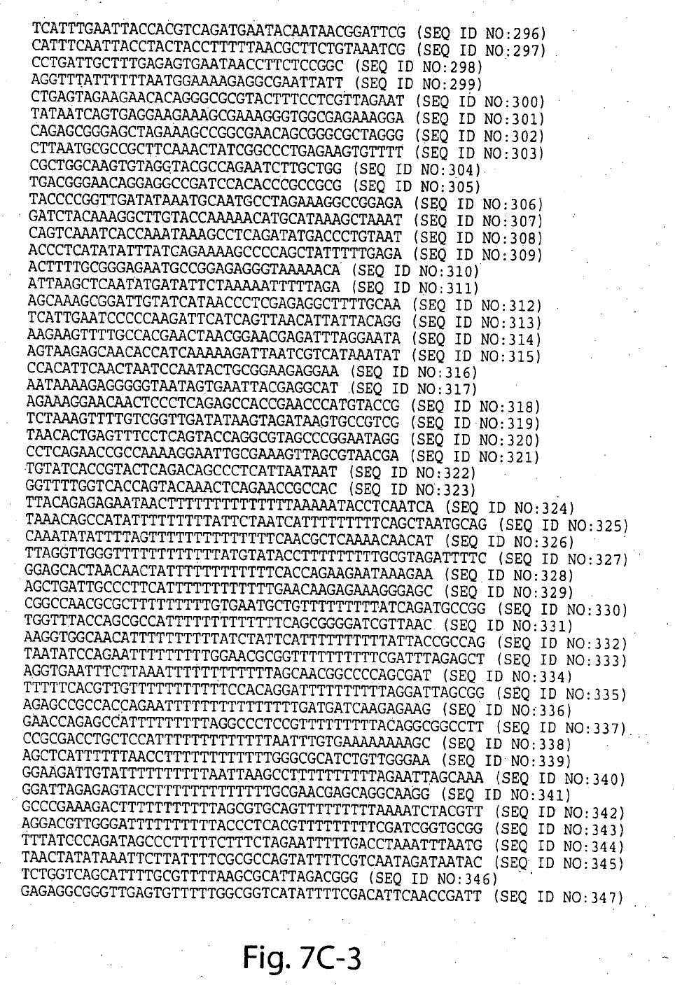

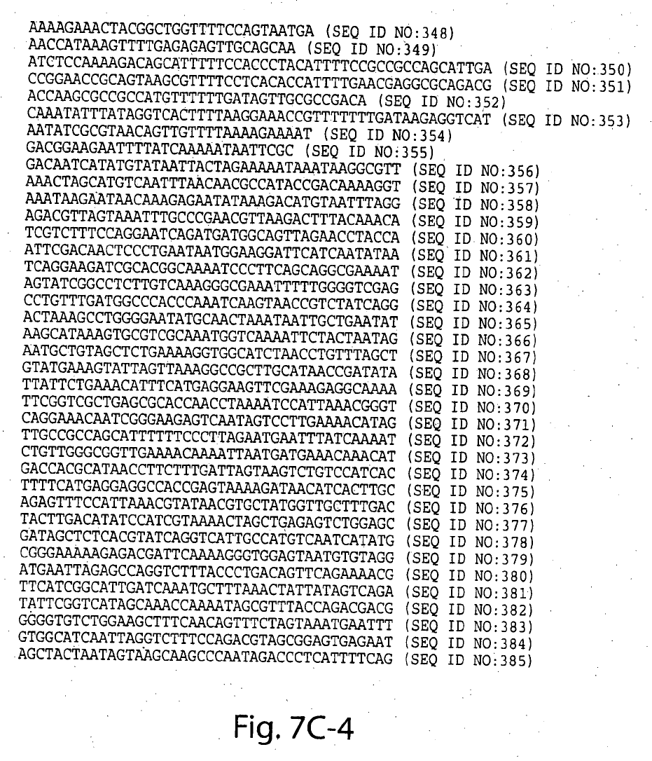

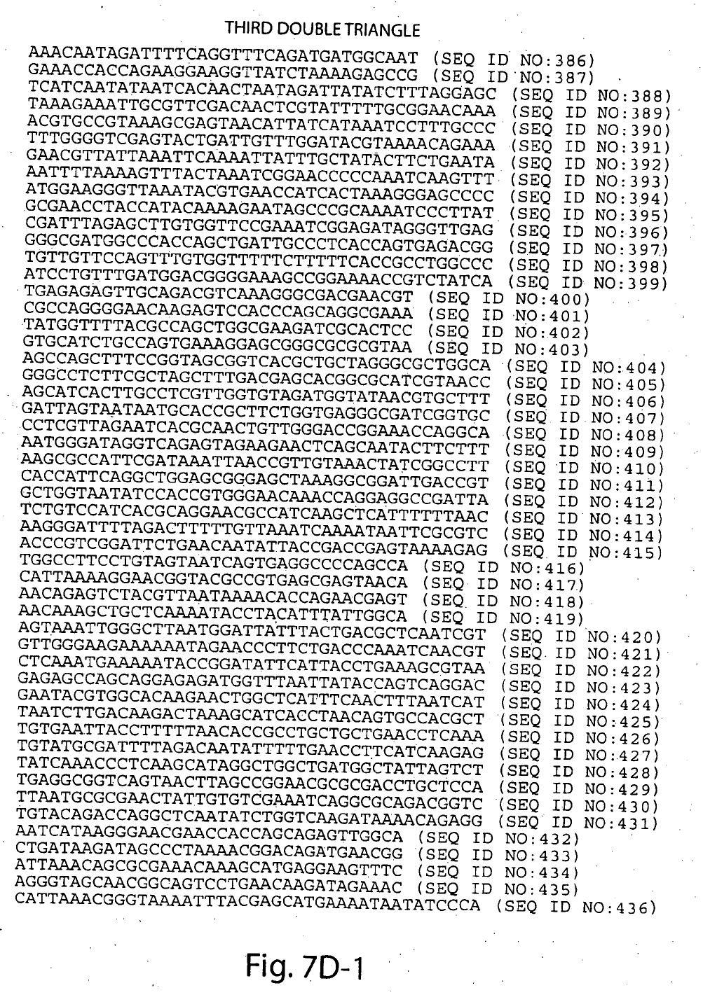

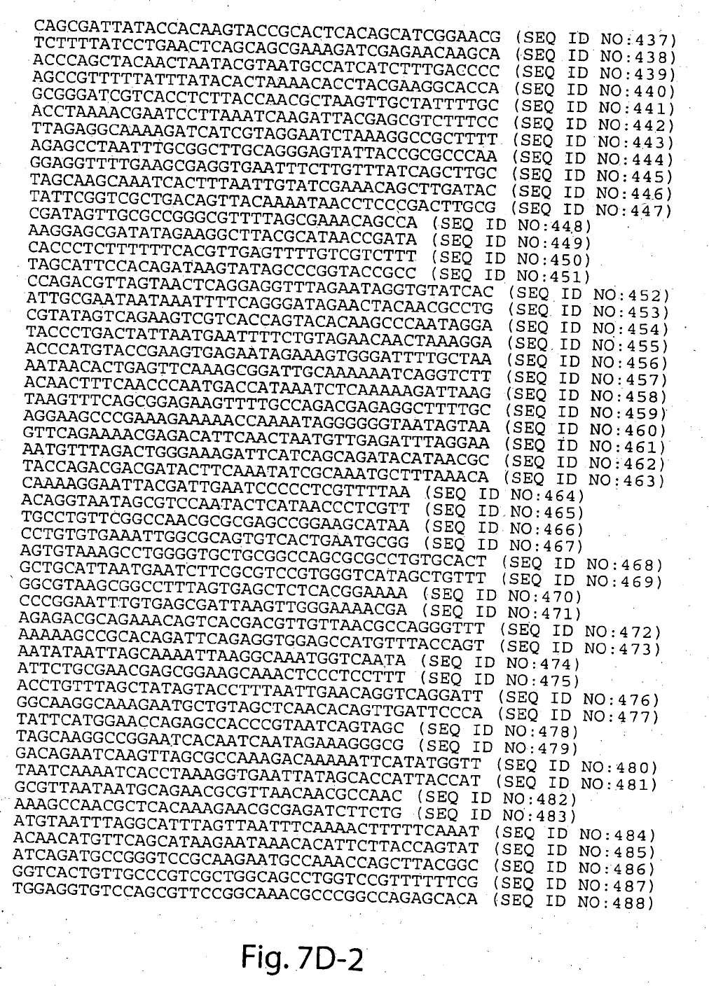

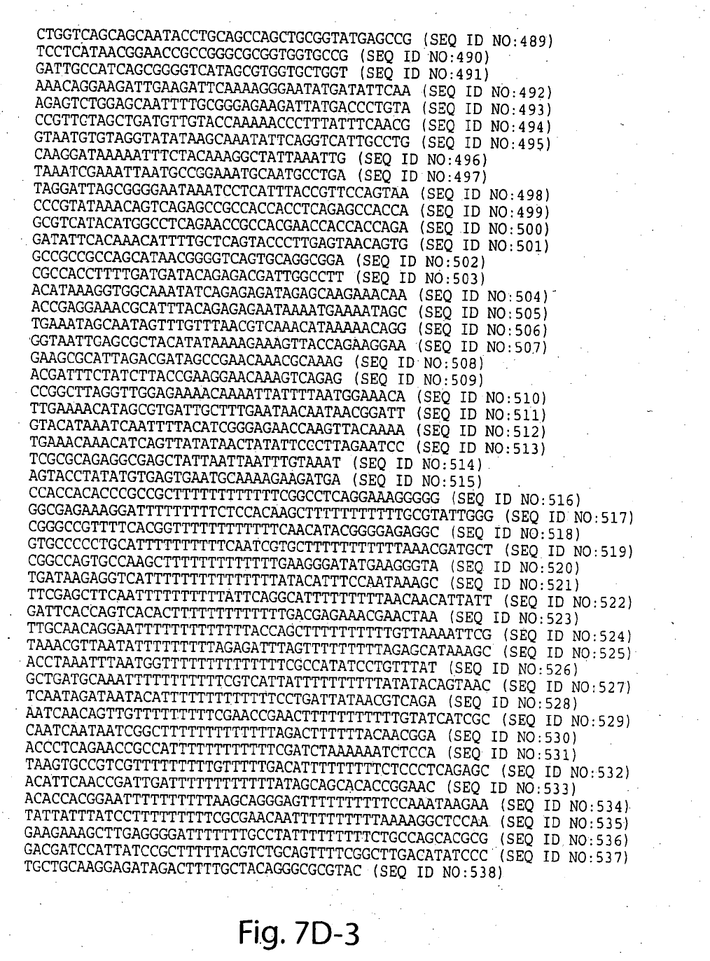

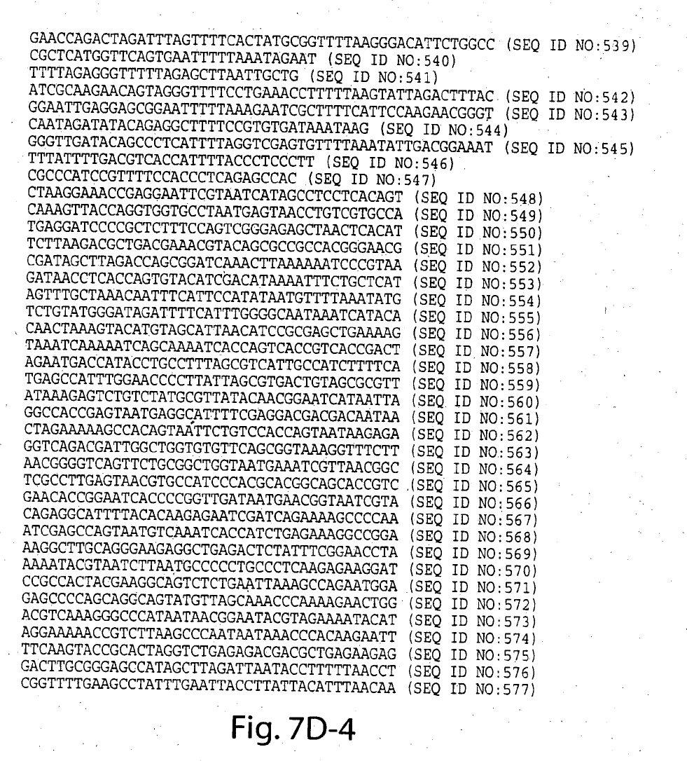

The scaffold nucleic acid sequence itself can be seen in FIG. 7 (SEQ ID NO: 1). The sequence was a p8100 scaffold sequence based on the m13mp18 backbone of a bacteriophage. In this example, to form a regular icosahedron, three copies of the m13mp18 backbone were folded in different ways using a plurality of unique nucleic acid stabilizers that would immobilize two or more portions of the scaffold nucleic acid sequence together. The three copies were further assembled together by using certain nucleic acid stabilizers associated with different copies that were complementary. The first set of sequences (SEQ ID NOs: 2-193) were used to fold the p8100 scaffold sequence into a first double-triangle shape (FIG. 7A), while the second set of sequences (SEQ ID NOs: 194-385) were used to fold the p8100 scaffold sequence into a second double-triangle shape (FIG. 7C) and a third set of sequences (SEQ ID NOs: 386-576) were used to fold the p8100 scaffold sequence into third double-triangle shape (FIG. 7D). Next, these three shapes were annealed together in a solution heated to about 50° C. Under these conditions, the shapes spontaneously aggregated to form a regular icosahedral shape.

### Example 6

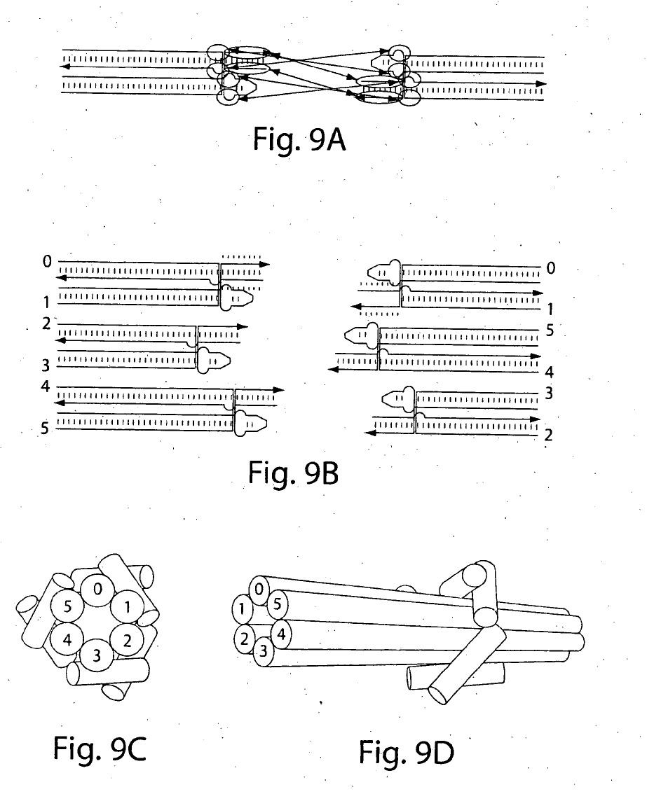

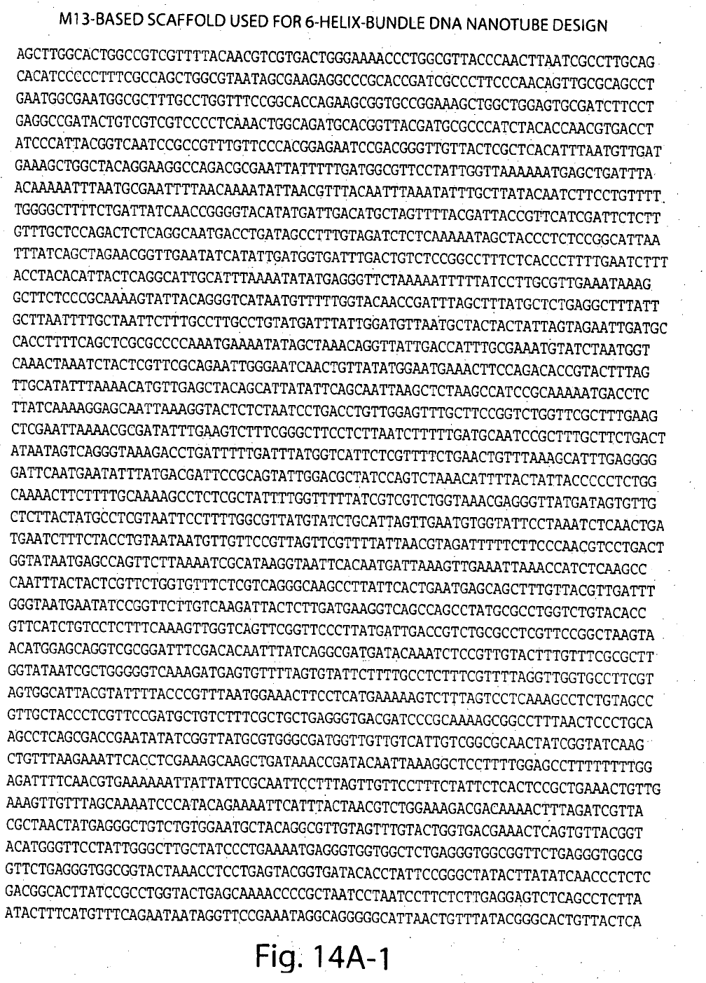

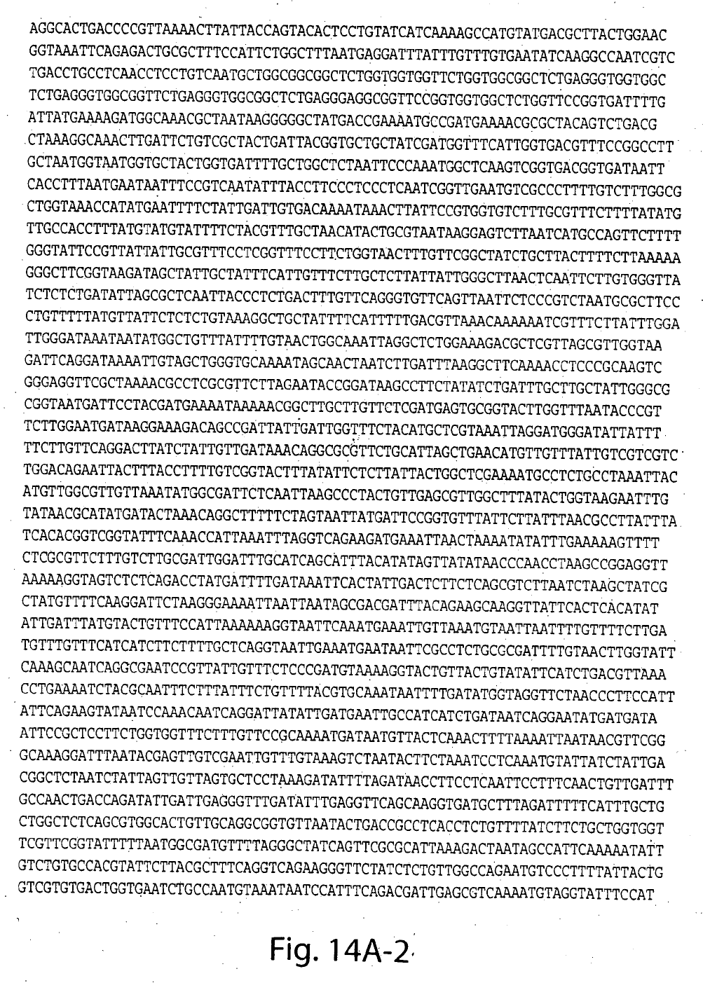

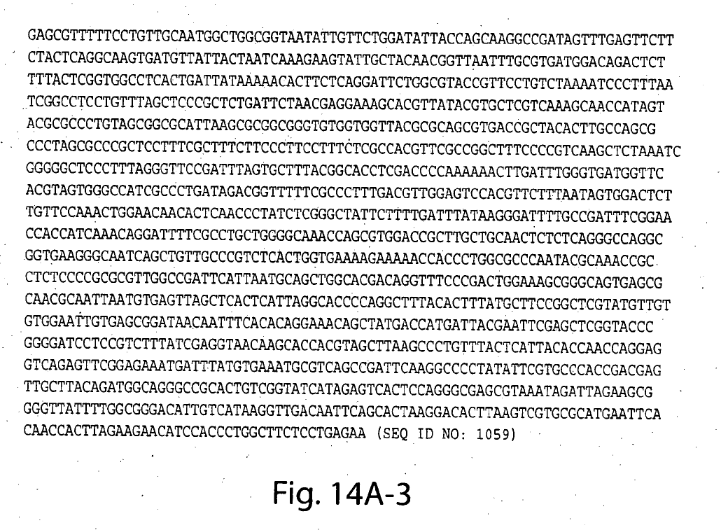

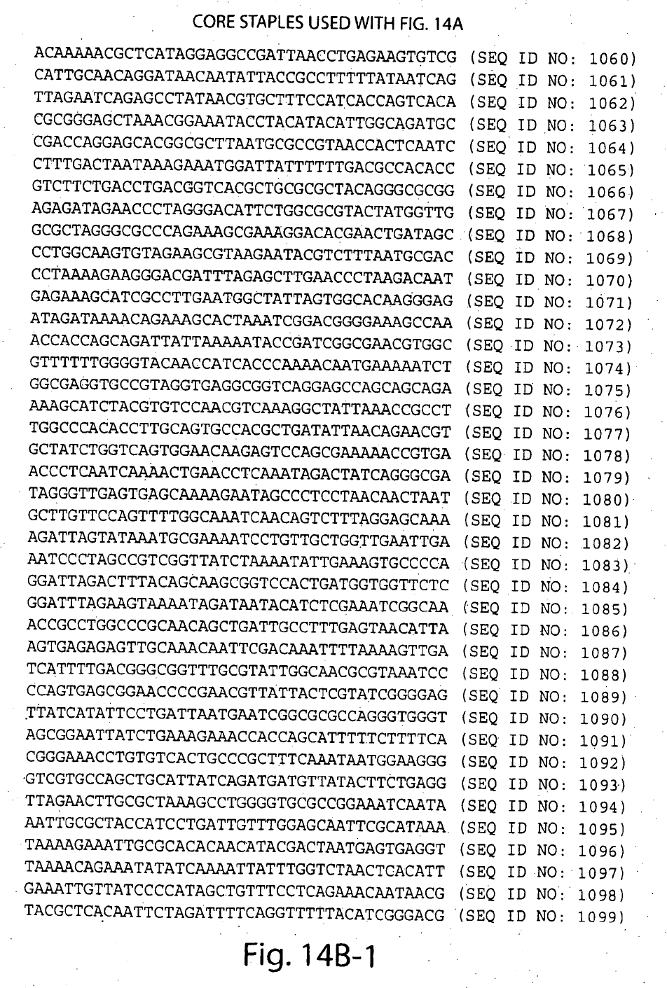

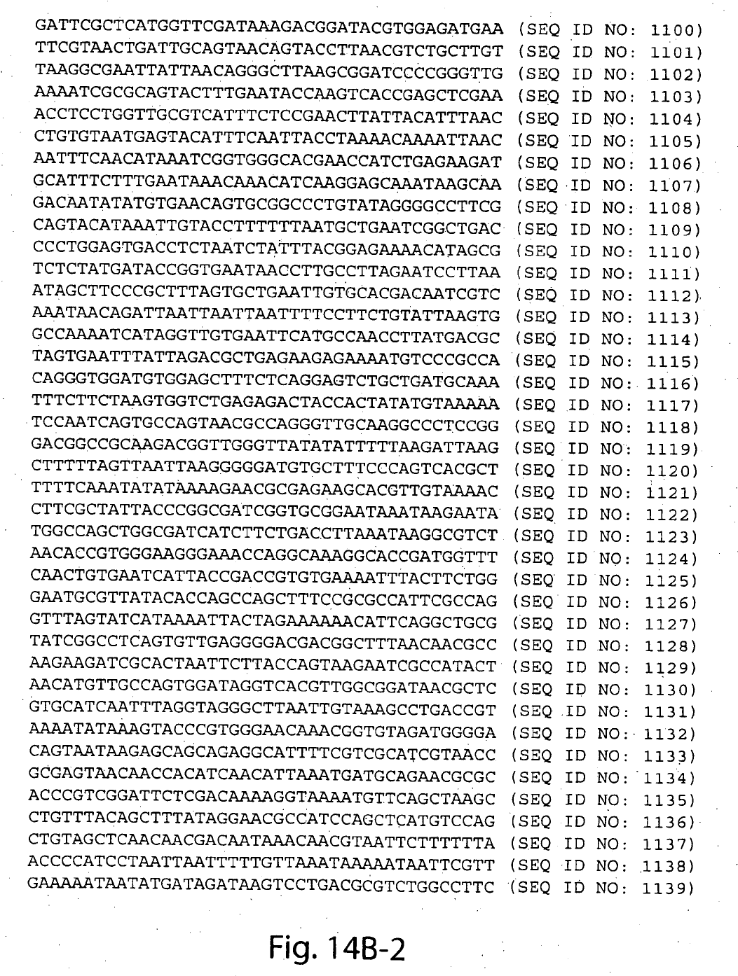

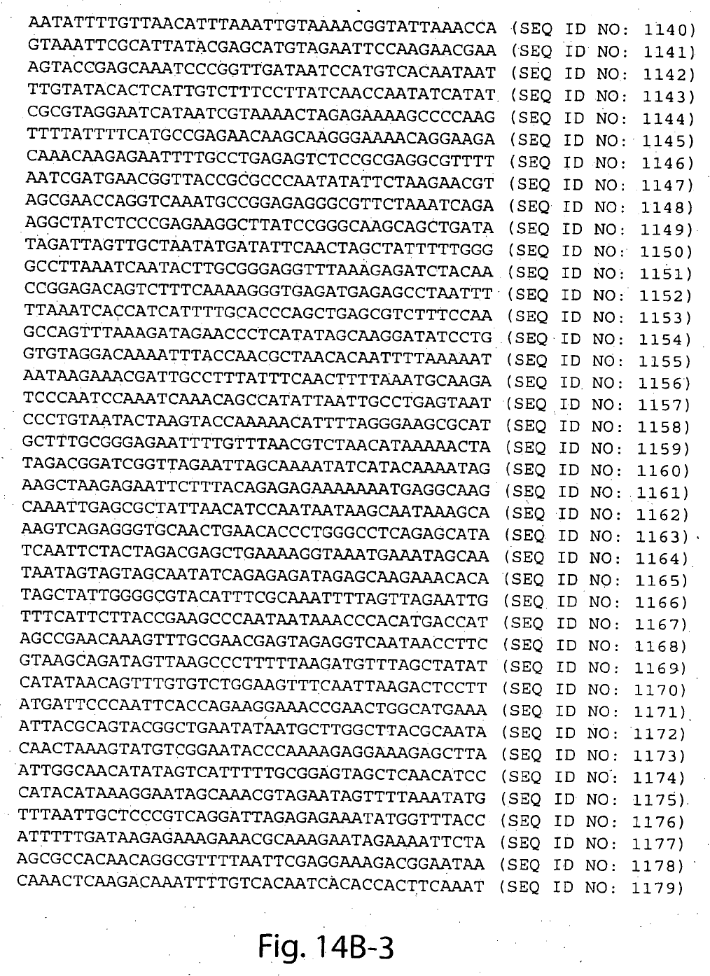

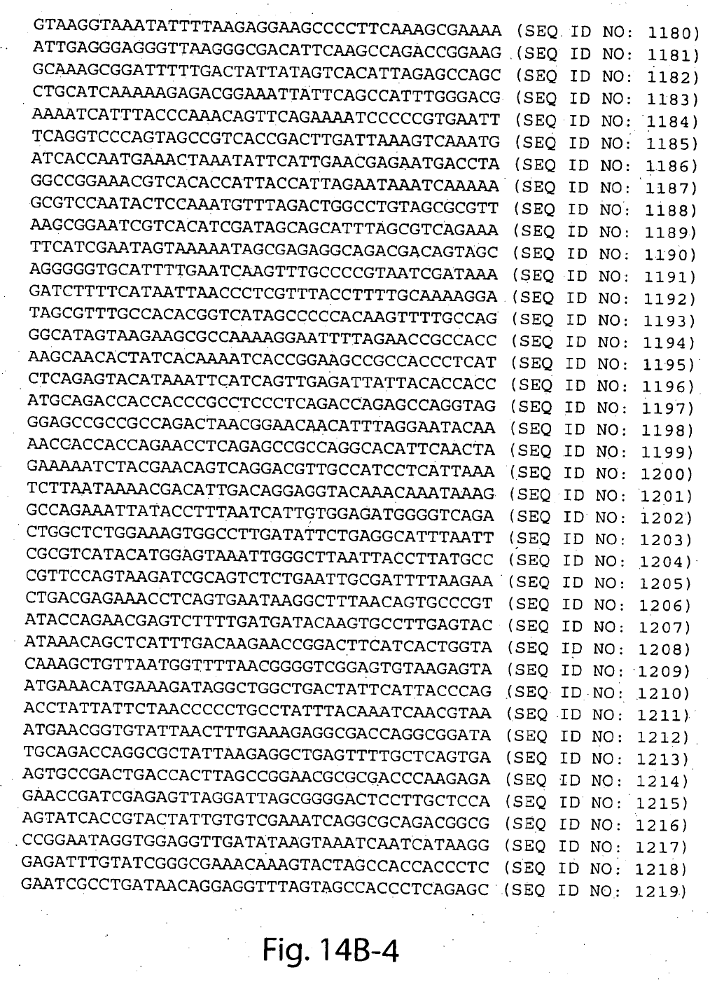

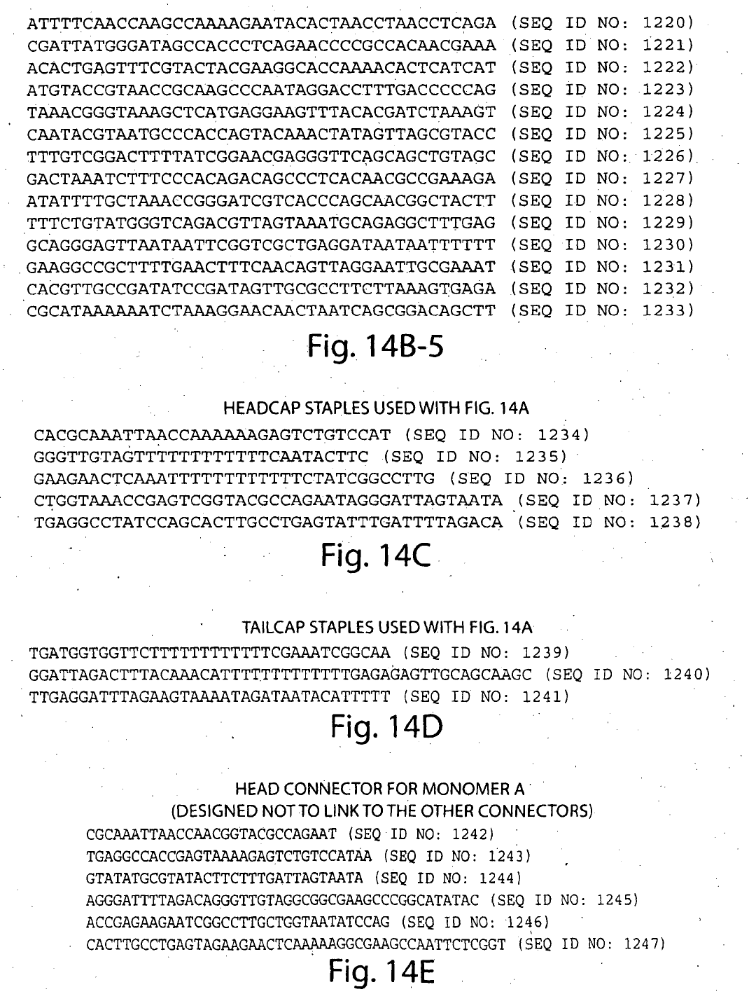

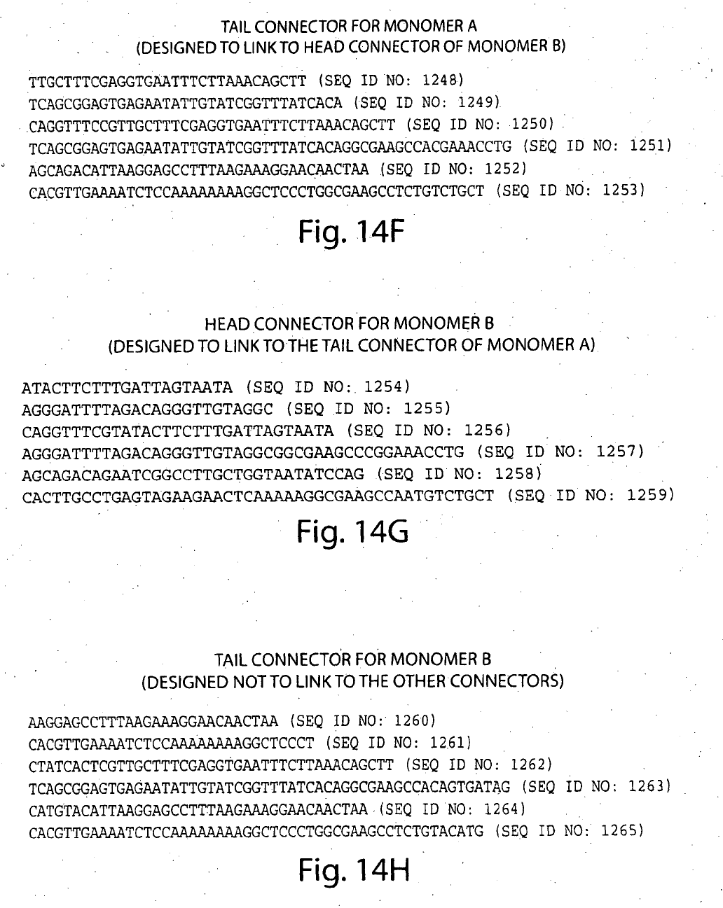

This example illustrates a joint between two nucleic acid portions, used in certain embodiments of the invention. The core architecture of the joint used in this example is outlined in FIG. 9A, with arrows and circles indicating substantial complementarity. This connectivity map shows how elements of two connectors come together to form new basepairs in a complete joint. This connectivity map appears complicated, but the underlying concept is similar to that shown in FIG. 8, i.e. the stems must denature and reanneal with strands from the opposing connector. FIG. 14 illustrates the sequences used in this example.

Two-base 3′ sticky ends protrude from three helices, while two-base 3′ recessed ends exist on the other three helices, as is shown in FIG. 9B; each of these uses the joint as outlined in FIG. 9A, in this example. Strand diagram for six-helix-bundle DNA-nanotube connectors. In this example, strands from helix 0 is not complementary to strand from helix 1 on the same connector, while helices 2, 3, 4, and 5 are protected.

After joint formation, the nicks lie on the outside of the helices orthogonal to the center of the six-helix-bundle DNA-nanotube. The single-strand extensions are arranged such that, after joint formation, an extra double helix lies against each of the double helices, arranged as shown in FIGS. 9C and 9D. Cylinders represent helices.

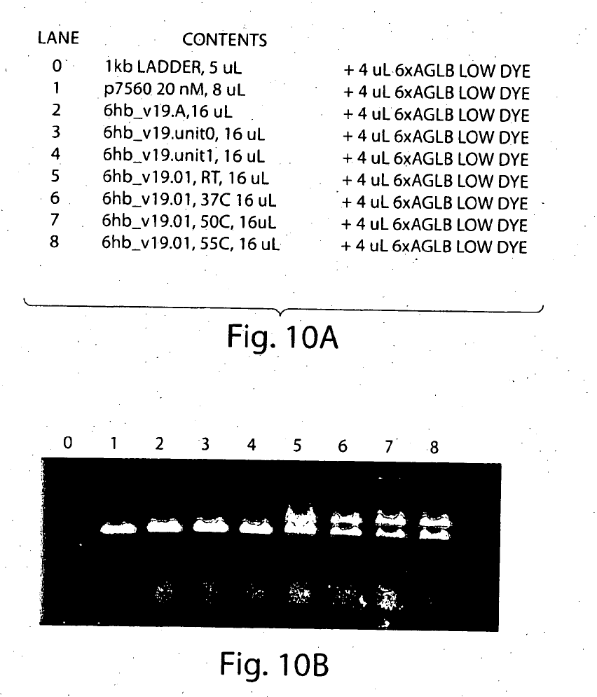

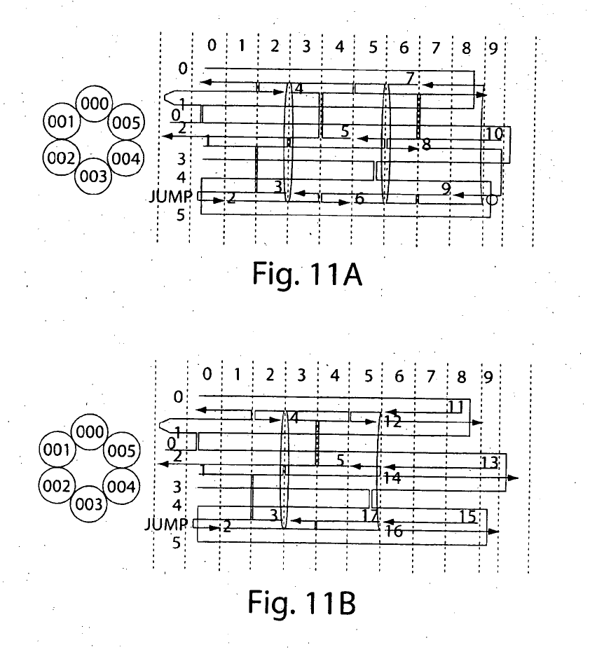

Verification that this stem-swap connector produces specific heterodimerization is provided in FIG. 10. The tail of a first nucleic acid was functionalized to link with the head of a second nucleic acid, while the head of the first nucleic acid and the tail of second nucleic acid were programmed to remain unlinked. The first nucleic acid was folded alone is shown in lane 3, and the second nucleic acid was folded alone is shown in lane 4. Both species remained nucleic acidic, demonstrating a lack of nonspecific linkage between head and tail of identical copies of nucleic acids. Upon mixing at varying temperatures for two hours (lanes 5 to 8), specific heterodimerization between the two nucleic acids was observed (temperatures used were 25° C., 37° C., 50° C., and 55° C. for lanes 5, 6, 7, and 8, respectively.

### Example 7

This example illustrates the formation of a wireframe icosahedron stabilized by the use of more than one stabilizer. In this example, the double helix of each fully formed strut contained 200 basepairs, compared to 210 basepairs per double helix in some of the above examples. Thus, the total number of staple-to-scaffold basepairs is 100×6×10=6000 basepairs. Include unpaired bases, each double-triangle monomer fit on an M13-based scaffold that is 7704 bases long. There are an average of two crossovers per 42 basepairs per helical interface for this design. The junctions (zones 7, 8, 9) impose 32 basepairs per three turns, as opposed to 42 basepairs per four turns in zones 0 to 6. This allows for the architecture of the connectors to be identical.

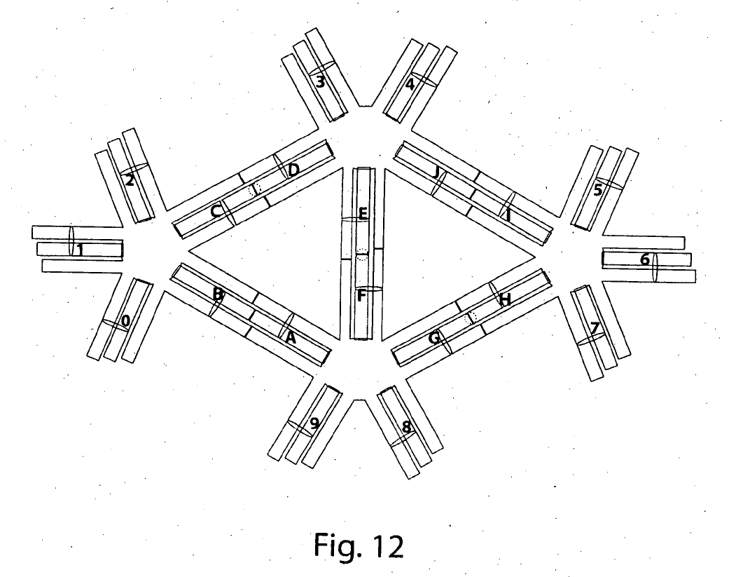

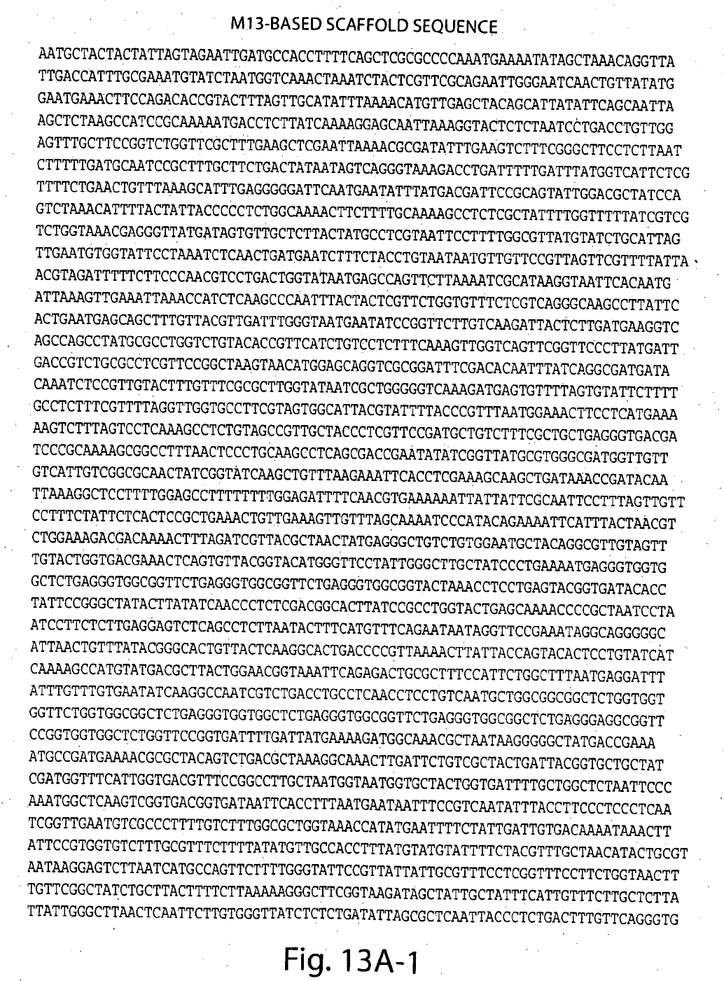

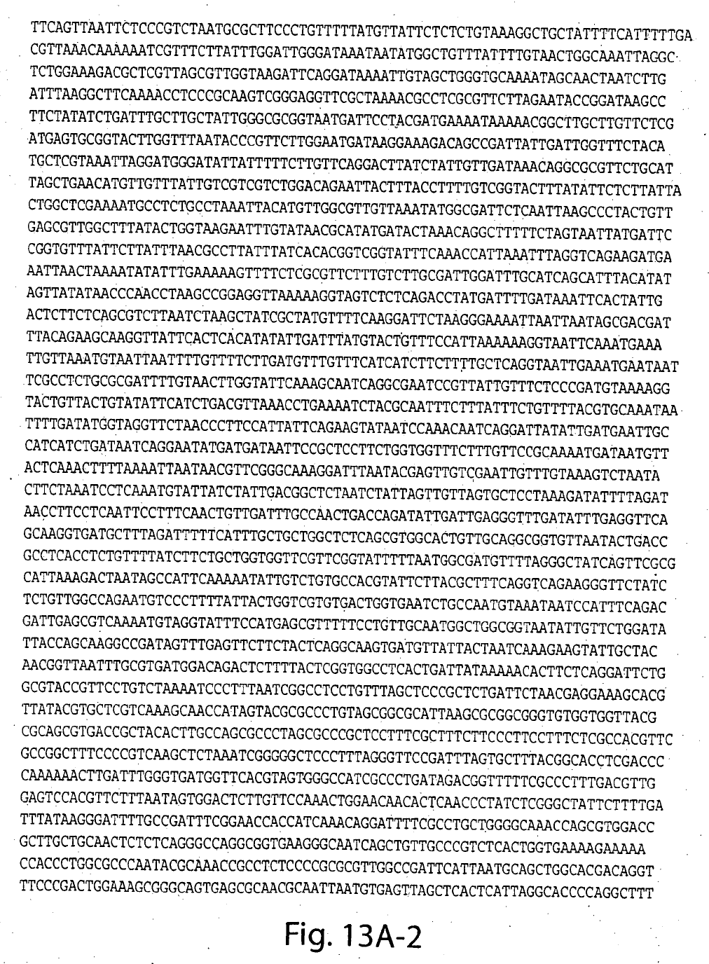

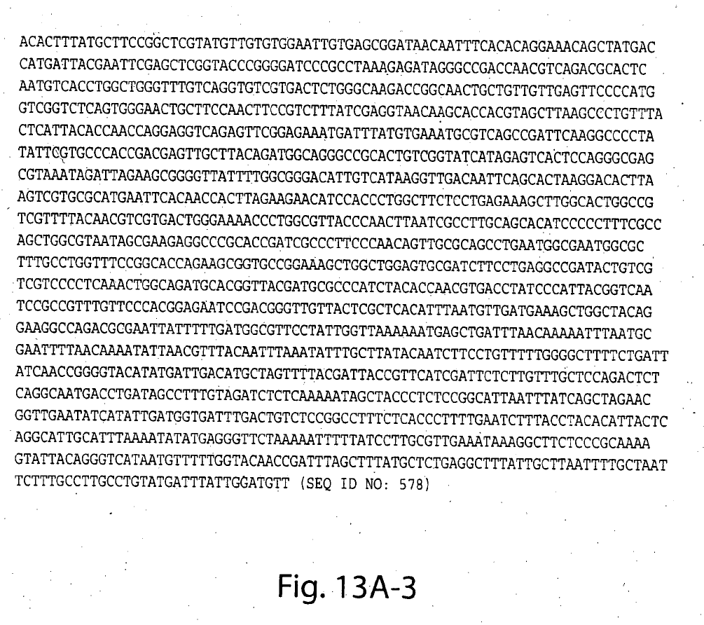

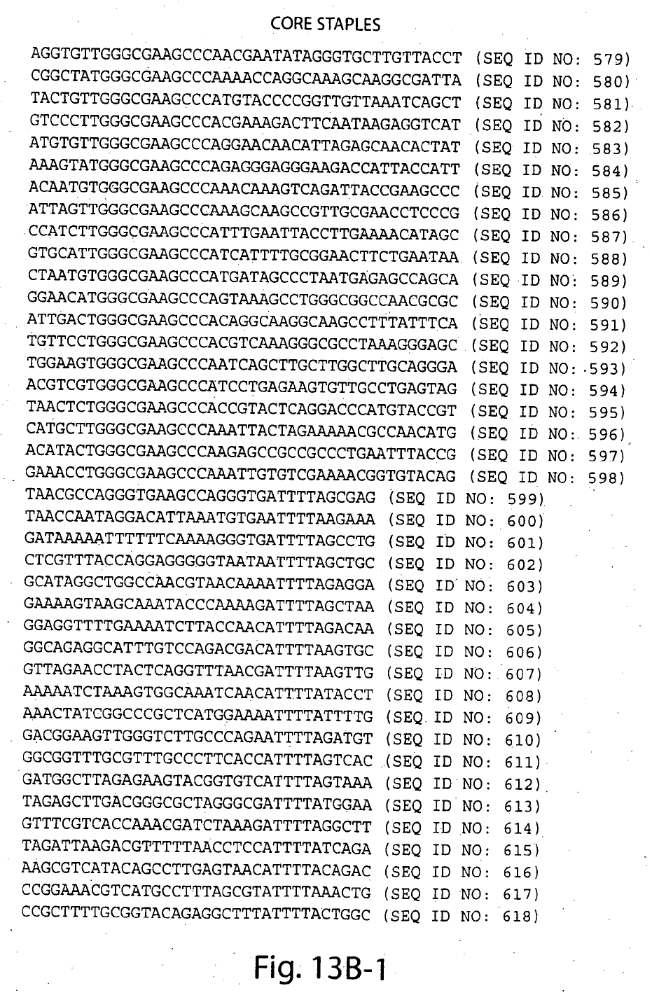

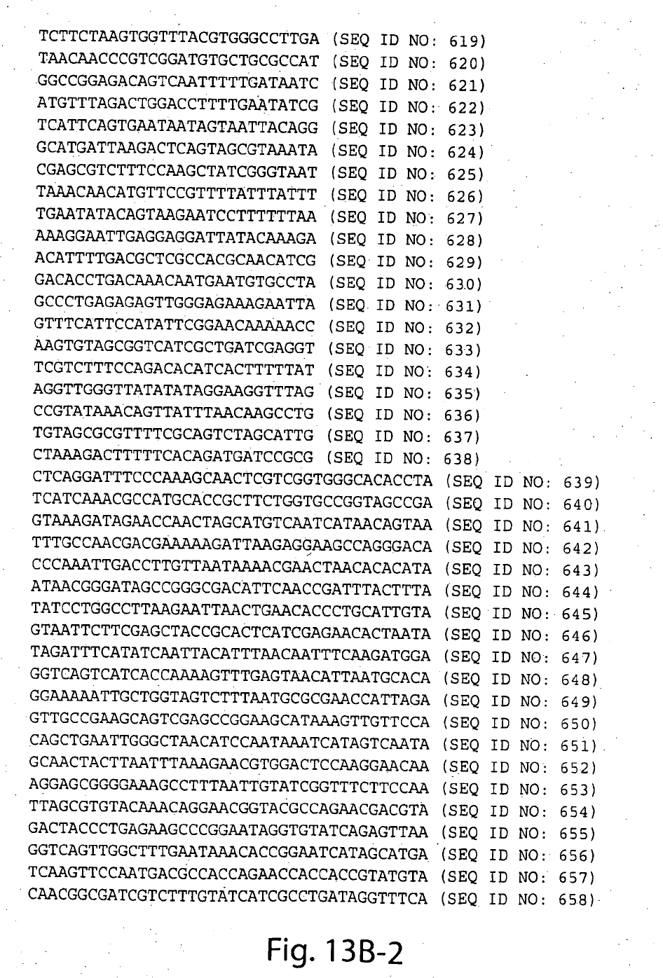

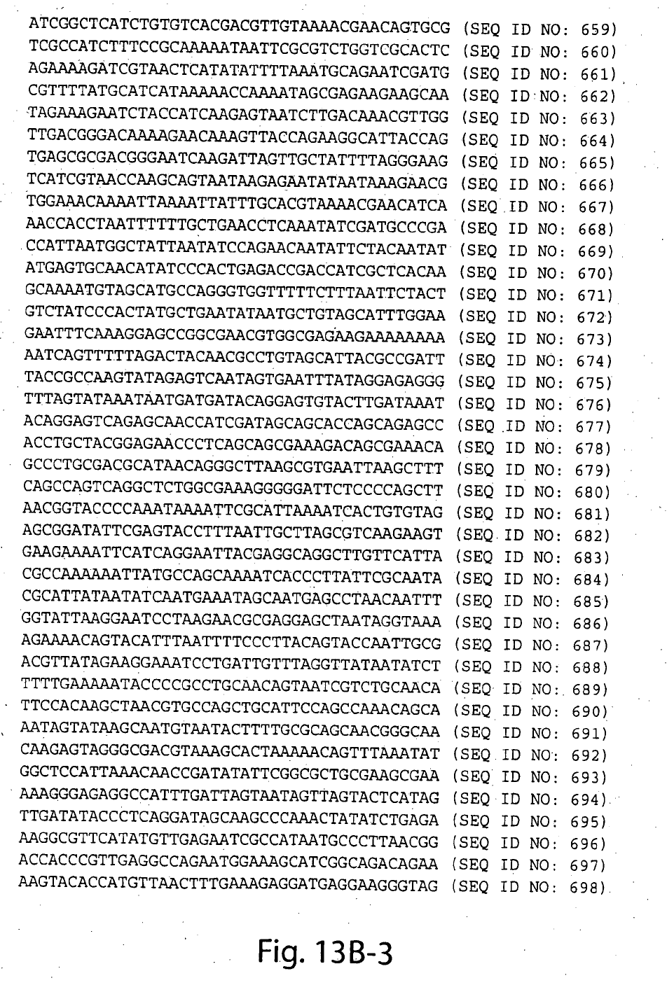

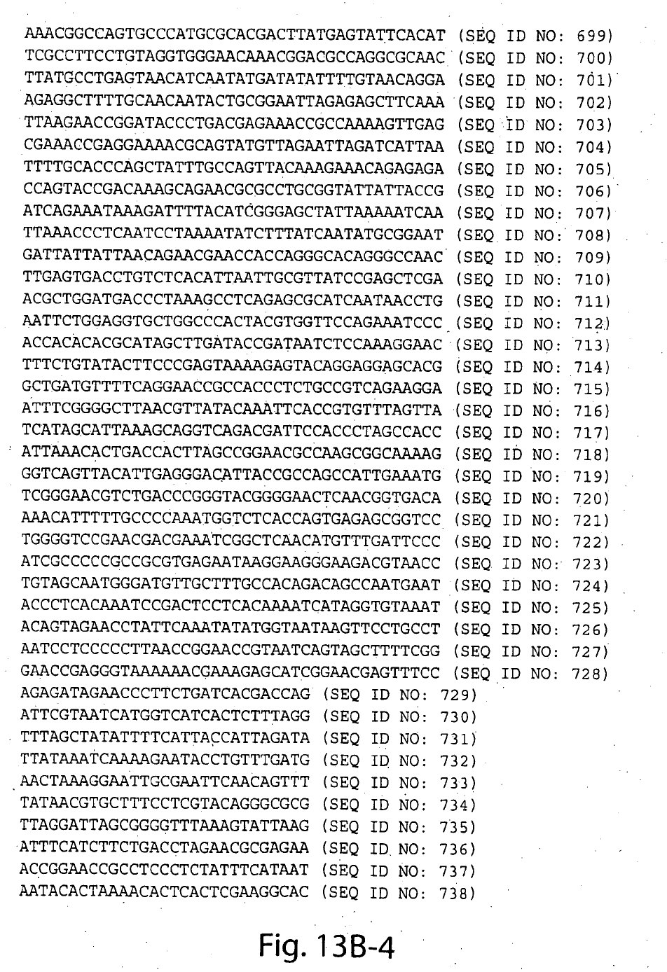

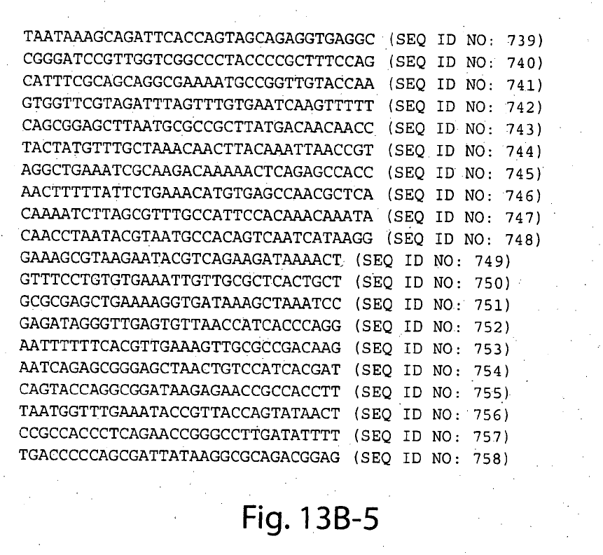

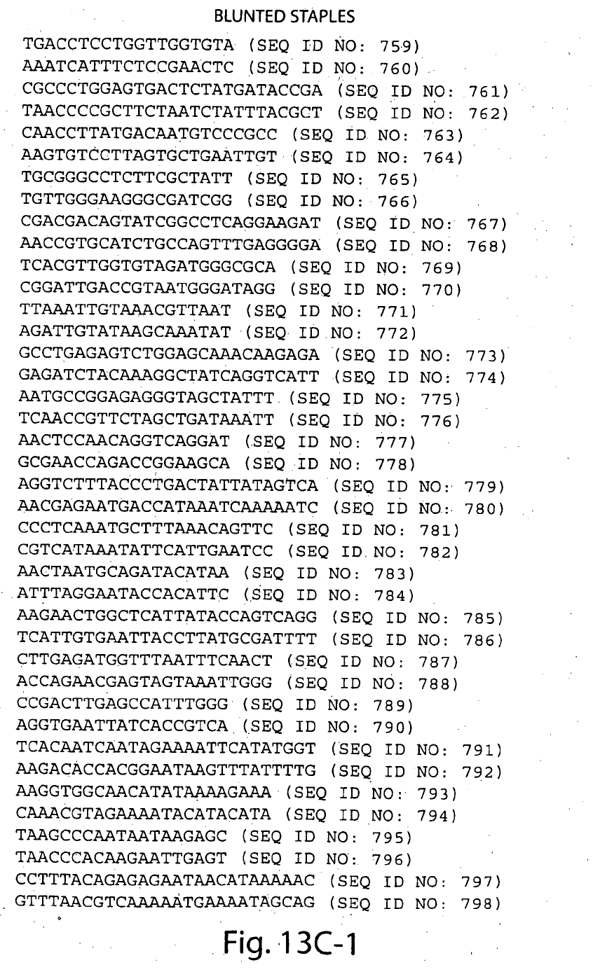

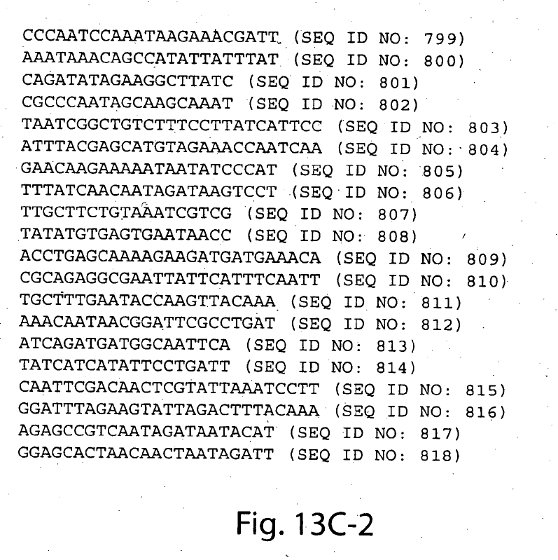

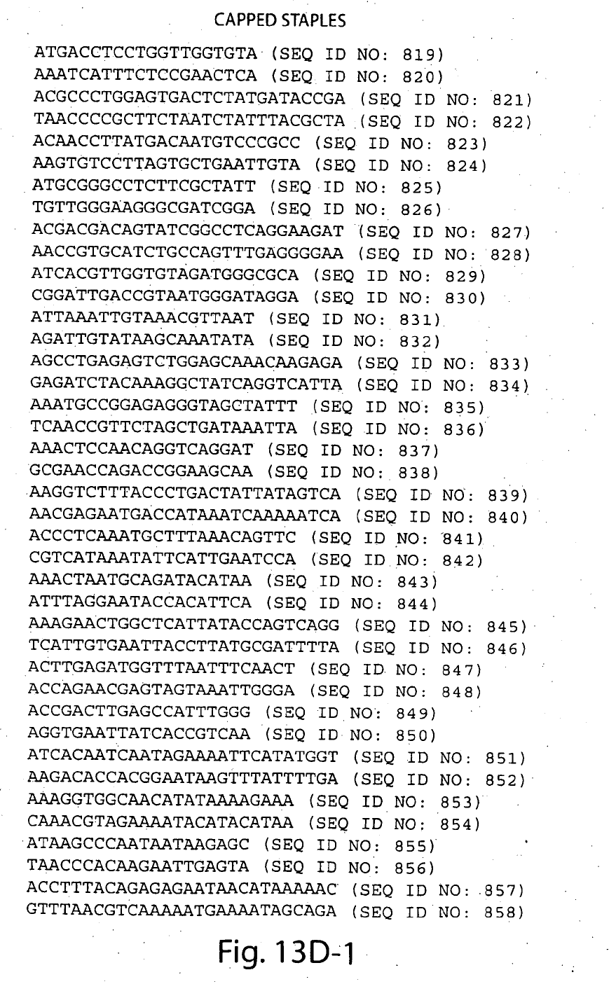

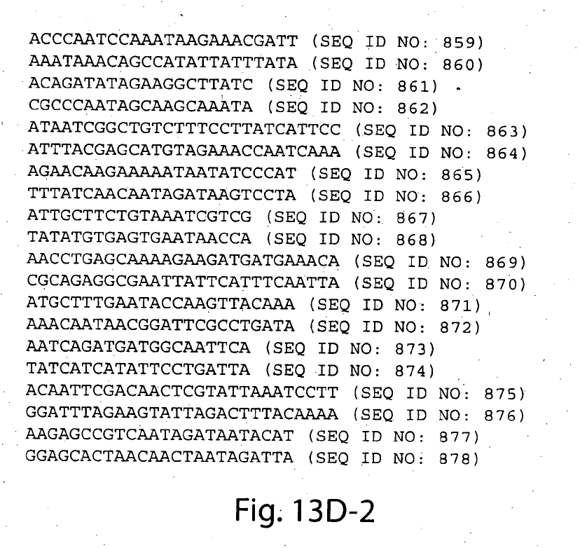

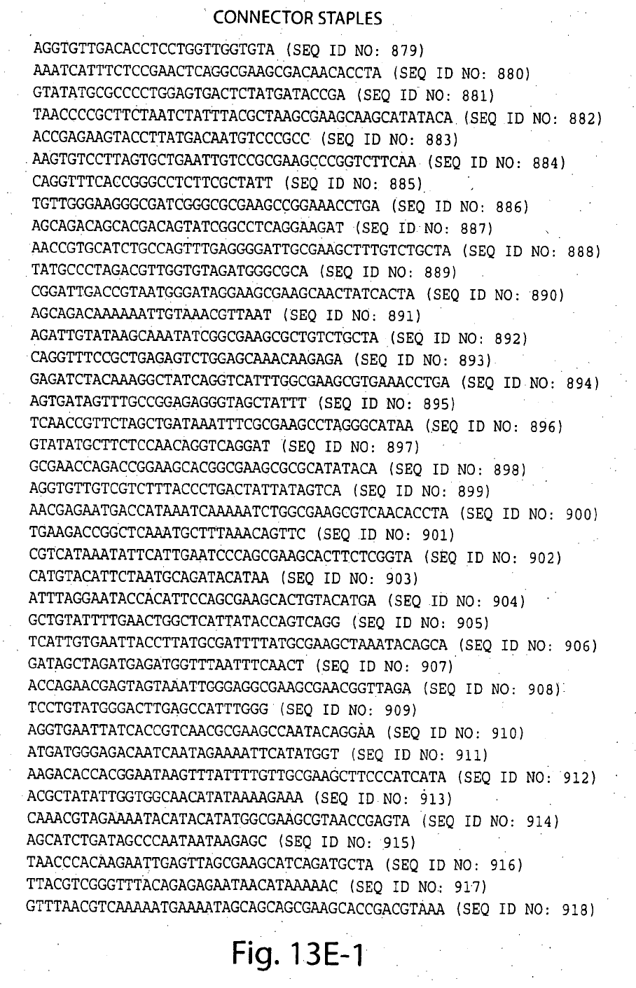

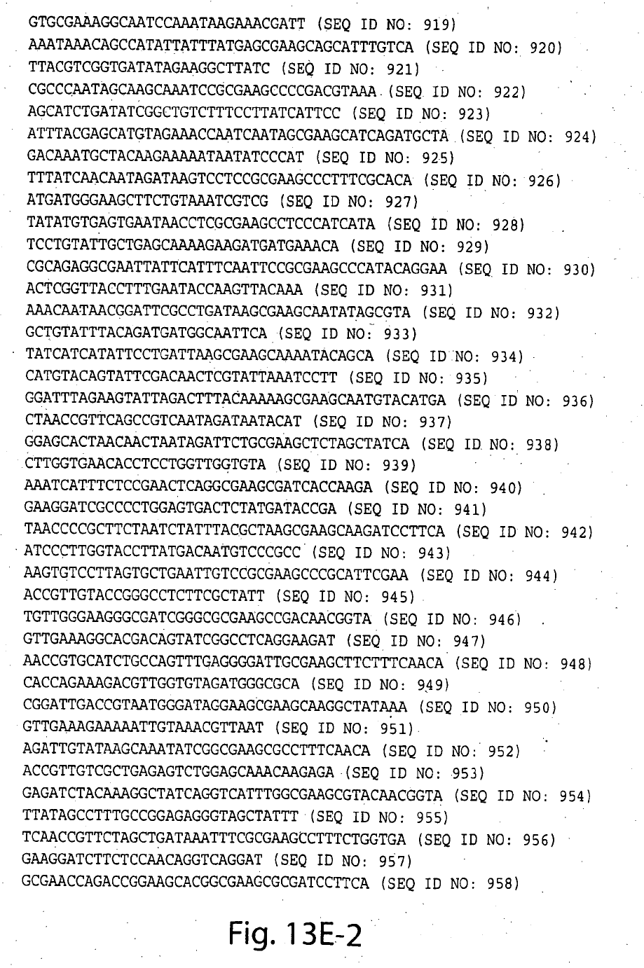

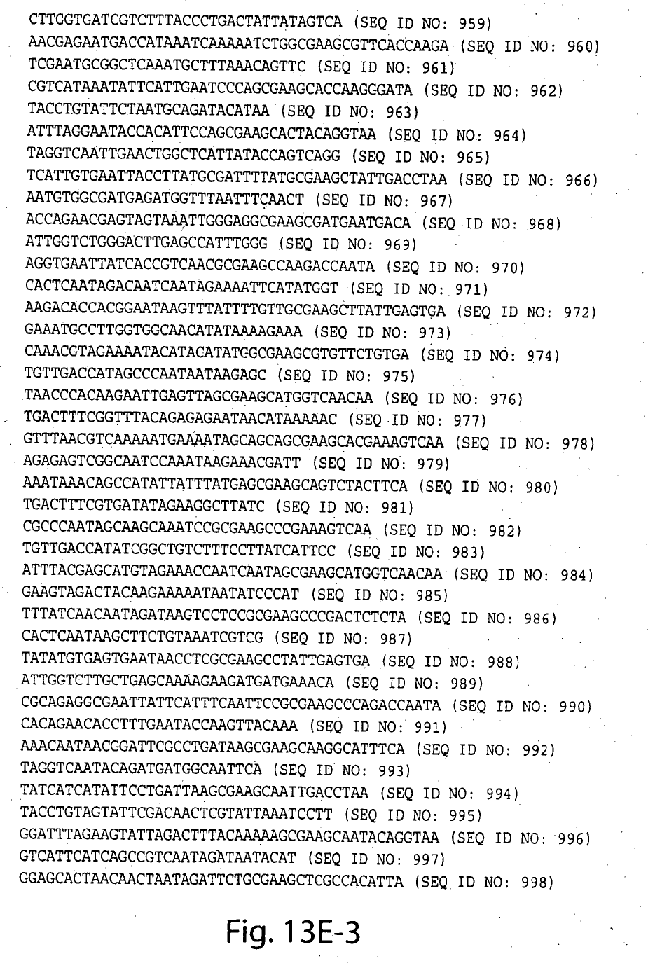

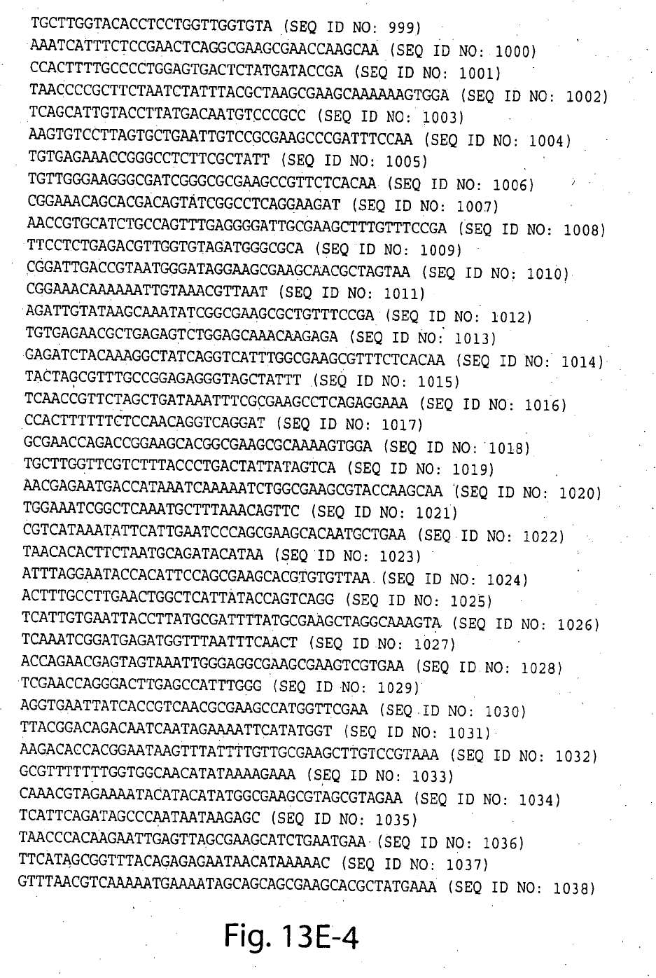

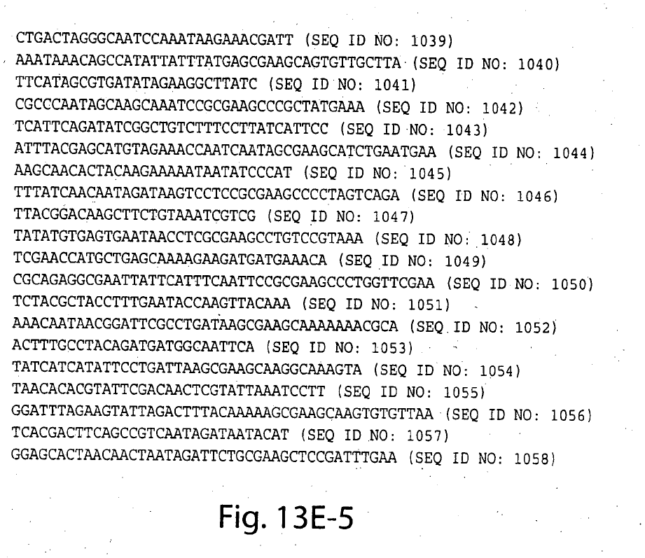

FIG. 11 shows this design; the sequences are shown in FIG. 13. FIG. 11A shows that the half-struts come together internally (e.g. E and F half-struts link in this way in FIG. 12). FIG. 11B shows half-struts that come together externally. FIG. 12 illustrates the layout of a “double-triangle” structure used to form the icosahedral structure.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A composition, comprising:
a molecular structure defining a plurality of vertices and pathways, at least some of the vertices having at least three pathways emanating therefrom, each of which connects two vertices, wherein at least one pathway connecting two vertices comprises a nanotube comprising nucleic acid.

2. The composition of claim 1, wherein the molecular structure is substantially rigid.

3-5. (canceled)

6. The composition of claim 1, wherein the molecular structure is formed from one or more nucleic acid strands at least some of which have a length of at least about 500 nucleotides.

7. The composition of claim 1, wherein the molecular structure is stabilized by one or more nucleic acid stabilizers, at least some of which comprise a nucleic acid having a length of less than about 100 nucleotides.

8-9. (canceled)

10. The composition of claim 1, wherein at least one pathway has a length of between the two vertices of at least about 40 nm.

11. (canceled)

12. The composition of claim 1, wherein at least one pathway connecting two vertices comprises a nanotube comprising nucleic acid.

13-14. (canceled)

15. The composition of claim 1, wherein at least one pathway connecting two vertices comprises a six-helix nucleic acid bundle of nucleic acid.

16. The composition of claim 1, wherein at least one pathway connecting two vertices comprises a ten-helix nucleic acid bundle of nucleic acid.

17. The composition of claim 1, wherein at least one vertex has at least four pathways emanating therefrom.

18-21. (canceled)

22. The composition of claim 1, wherein the molecular structure comprises at least four vertices.

23-27. (canceled)

28. The composition of claim 1, wherein the molecular structure defines a three-dimensional interior space.

29. (canceled)

30. The composition of claim 1, wherein the molecular structure has a smallest dimension that is at least about 100 nm.

31. The composition of claim 1, wherein each vertex of the molecular structure is connected to at least two other vertices that are connected to each other.

32. The composition of claim 1, wherein the plurality of vertices defines faces of the molecular structure, wherein each face of the molecular structure is defined by only three vertices.

33-42. (canceled)

43. A composition, comprising:
a molecular structure defining a plurality of vertices and pathways, at least some of the vertices having at least three pathways emanating therefrom, each of which connects two vertices, wherein at least one pathway connecting two vertices comprises a nucleic acid and has a length between the two vertices of at least about 40 nm.

44. (canceled)

45. The composition of claim 43, wherein the molecular structure is stabilized by one or more nucleic acid stabilizers at least some of which comprise a nucleic acid having a length of less than about 100 nucleotides.

46. (canceled)

47. The composition of claim 43, wherein at least one pathway connecting two vertices comprises a nanotube comprising nucleic acid.

48. The composition of claim 43, wherein the molecular structure defines a three-dimensional interior space.

49. The composition of claim 43, wherein the molecular structure has a smallest dimension that is at least about 100 nm.

50-76. (canceled)

77. A composition, comprising:
a molecular structure defining a three-dimensional interior space, the molecular structure being formed from one or more nucleic acids and having a smallest dimension of at least about 100 nm.

78-118. (canceled)
